(19) United States
(12) Patent Application Publication
(10) Pub. No.: US 2025/0032605 A1
(43) Pub. Date: Jan. 30, 2025

(54) RATIONALLY DESIGNED IMMUNOGENS

(71) Applicants: The General Hospital Corporation, Boston, MA (US); President and Fellows of Harvard College, Cambridge, MA (US)

(72) Inventors: Aaron G. SCHMIDT, Boston, MA (US); Blake M. HAUSER, Boston, MA (US); Timothy M. CARADONNA, Boston, MA (US)

(21) Appl. No.: 18/715,905

(22) PCT Filed: Dec. 5, 2022
(86) PCT No.: PCT/US2022/080950

§ 371 Date: Jun. 3, 2024

Related U.S. Application Data
(60) Provisional application No. 63/319,724, filed on Mar. 14, 2022.
(60) Provisional application No. 63/286,003, filed on Dec. 4, 2021.

(51) Int. Cl. A61K 39/215; A61K 39/00; A61P 31/14; C07K 14/005; C12N 7/00

(52) U.S. Cl. CPC A61K 39/215; A61P 31/14; C07K 14/005; C12N 7/00; A61K 2039/55505; A61K 2039/70; C12N 2770/00; C12N 2770/20034

(57) ABSTRACT

The invention relates to a monomeric immunogen comprising one receptor binding domain of a Coronavirus spike protein, wherein the receptor binding domain includes at least 4 non-native. N-linked, glycosylation sites.

### CROSS-REFERENCE TO RELATED APPLICATIONS

This application claims benefit of 63/319,724, filed Mar. 14, 2022, and 63/286,003, filed Dec. 4, 2021, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Dec. 5, 2022, is named “51640-002WO3_Sequence_Listing_12_5_22” and is 90,915 bytes in size.

### BACKGROUND OF THE INVENTION

The invention relates to compositions and methods for treating viral infections.

The emergence of SARS-CoV-2 (SARS-2) and the subsequent global pandemic have highlighted the disruptive threat posed by viruses for which humans have no prior immunity. Rapid vaccine development has led to an unprecedented number of candidates already in Phase 3 clinical trials or approved since January 2020. While differing in platform (e.g., mRNA, adenovirus, nanoparticle), the primary immunogen is the SARS-2 spike ectodomain. With the continued global spread of SARS-2, in conjunction with potential vaccinations, it is likely that a large proportion of the global population will eventually develop an immune response to SARS-2. However, even after potentially achieving herd immunity sufficient to slow its spread, further SARS-2 evolution leading to variants that escape immunity as well as emerging novel coronaviruses with pandemic potential remain a concern. Indeed, surveillance efforts have identified numerous unique coronaviruses within different animal reservoirs, raising the possibility of zoonotic transmission. Such events are likely to increase in frequency as a result of human impact on the environment. While the current SARS-2 pandemic has an estimated infection fatality rate of between ˜1-2%, previous SARS-CoV (SARS-1) and MERS-CoV (MERS) outbreaks had higher lethality with ˜10% and ˜35% case fatality rates, respectively, raising the possibility that future novel coronaviruses may have high mortality. Additionally, elicited immunity to SARS-2 infection may not protect against emergent novel coronaviruses that are closely related to SARS-2.

Accordingly, there is a need to develop vaccines that confer potential pan-coronavirus immunity.

### SUMMARY OF THE INVENTION

In one aspect, the invention features a monomeric immunogen including one receptor binding domain of a Coronavirus spike protein, wherein the receptor binding domain includes at least 1, 2, 3, or 4 or more non-native, N-linked, glycosylation sites. In preferred embodiments, the receptor binding domain includes 4 non-native, N-linked, glycosylation sites. In other preferred embodiments, the receptor binding domain includes 5 non-native, N-linked, glycosylation sites. In still other preferred embodiments, the receptor binding domain includes 6 non-native, N-linked, glycosylation sites (for example, as is described herein for the SARS-2hg construct). In still other preferred embodiments, the receptor binding domain includes 7, 8, 9 or more non-native, N-linked, glycosylation sites (for example, as is described herein for SEQ ID NO: 11).

In some embodiments, the receptor binding domain of a Coronavirus spike protein includes a SARS-CoV-2, SARS-1, WIV1, MERS, Rs_672, Rp_Shaanxi2011, Cp_Yunnan2011, BtRf-HeB2013, BtRf-SX2013, BtRs-HuB2013, BtRs-GX2013, BtRs-YN2013, Longquan-140, YNLF_31C, YNLF_34C, As6526, Rs4081, Rs4237, Rs4247, Rs4255, BtRI_SC2018, BtRs_YN2018A, BtRS_YN2018C, BtRs_YN2018D, HKU9, HKU3-1, HKU4, HKU5, RaTG13, or SHC014 receptor binding domain. In some embodiments, the receptor binding domain includes a heterologous receptor binding motif. In yet other embodiments, the heterologous receptor binding motif includes at least 1 or 2 or more non-native, N-linked, glycosylation sites. In yet other embodiments, the heterologous receptor binding motifs do not necessarily require engineered glycosylation sites.

In some embodiments, the heterologous receptor binding motif of a Coronavirus is the ACE2 receptor binding motif of SARS-CoV-2 or a variant thereof. In one embodiment, the ACE2 receptor binding motif amino acid sequence includes

(SEQ ID NO: 1)
NSNNLDSKVGGNYNYLYRLFRKSNLKPFERDISTEIYQAGSTPCN
GVEGFNCYFPLQSYGFQPTNGVGYQPY.

In some embodiments, the SARS-CoV-2 variant is Alpha (B.1.1.7, Q.1-Q.8), Beta (B.1.351, B.1.351.2, B.1.351.3), Gamma (P.1, P.1.1, P.1.2), Epsilon (B.1.427, B.1.429), Eta (B.1.525), lota (B.1.526), Kappa (B.1.617.1), Zeta (P.2), Mu (B.1.621, B.1.621.1) or Delta (B.1.617.2, AY.1 sublineages).

In some embodiments, the SARS-CoV-2 variant is Alpha (B.1.1.7, Q.1-Q.8) and the ACE2 receptor binding motif amino acid sequence includes

(SEQ ID NO: 2)
NSNNLDSKVGGNYNYLYRLFRKSNLKPFERDISTEIYQAGSTPCN
GVEGFNCYFPLQSYGFQPTYGVGYQPY.

In some embodiments, the SARS-CoV-2 variant is Beta (B.1.351, B.1.351.2, B.1.351.3), and the ACE2 receptor binding motif amino acid sequence

(SEQ ID NO: 3)
NSNNLDSKVGGNYNYLYRLFRKSNLKPFERDISTEIYQAGSTPCN
GVKGFNCYFPLQSYGFQPTYGVGYQPY.

In some embodiments, the SARS-CoV-2 variant is Gamma (P.1, P.1.1, P.1.2), and the ACE2 receptor binding motif amino acid sequence includes

(SEQ ID NO: 4)
NSNNLDSKVGGNYNYLYRLFRKSNLKPFERDISTEIYQAGSTPCN
GVKGFNCYFPLQSYGFQPTYGVGYQPY.

In some embodiments, the SARS-CoV-2 variant is Epsilon (B.1.427, B.1.429), and the ACE2 receptor binding motif amino acid sequence includes

(SEQ ID NO: 5)
NSNNLDSKVGGNYNYRYRLFRKSNLKPFERDISTEIYQAGSTPCN
GVEGFNCYFPLQSYGFQPTNGVGYQPY.

In some embodiments, the SARS-CoV-2 variant is Eta (B.1.525), and the ACE2 receptor binding motif amino acid sequence includes

(SEQ ID NO: 6)
NSKNLDSKVGGNYNYLFRLFRKSNLKPFERDISTEIYQAGSTPCN
GVKGFNCYFPLQSYGFQPTNGVGYQPY.

In some embodiments, the SARS-CoV-2 variant is lota (B.1.526), and the ACE2 receptor binding motif amino acid sequence includes

(SEQ ID NO: 7)
NSNNLDSKVGGNYNYLYRLFRKSNLKPFERDISTEIYQAGSTPCN
GVKGFNCYFPLQSYGFQPTNGVGYQPY.

In some embodiments, the SARS-CoV-2 variant is Kappa (B.1.617.1), and the ACE2 receptor binding motif amino acid sequence includes

(SEQ ID NO: 8)
NSNNLDSKVGGNYNYRYRLFRKSNLKPFERDISTEIYQAGSTPCN
GVQGFNCYFPLQSYGFQPTNGVGYQPY.

In some embodiments, the SARS-CoV-2 variant is Zeta (P.2), Mu (B.1.621, B.1.621.1), and the ACE2 receptor binding motif amino acid sequence includes

(SEQ ID NO: 9)
NSNNLDSKVGGNYNYLYRLFRKSNLKPFERDISTEIYQAGSTPCN
GVKGFNCYFPLQSYGFQPTNGVGYQPY.

In some embodiments, the SARS-CoV-2 variant is Delta (B.1.617.2, AY.1), and the ACE2 receptor binding motif amino acid sequence includes

(SEQ ID NO: 10)
NSNNLDSKVGGNYNYRYRLFRKSNLKPFERDISTEIYQAGSKPCN
GVEGFNCYFPLQSYGFQPTNGVGYQPY.

In some embodiments, the receptor binding domain of a Coronavirus spike protein includes a receptor binding domain selected from a SARS-1 or WIV1 receptor binding domain and the heterologous receptor binding motif of a Coronavirus is the ACE2 receptor binding motif of SARS-CoV-2.

In some embodiments, the receptor binding motif includes a receptor binding motif shown in Table 3.

In another aspect, the invention features a dimeric immunogen comprising two of the monomeric immunogens of the foregoing aspect (for example, those described in Example 5). In some embodiments, the dimer is two monomers of rsWIV1. In some embodiments, the dimer is two monomers of rsSARS-1. In other embodiments, the dimer is two monomers of rsYNLF-34C. In some embodiments, the dimer is two different monomers.

In another aspect, the invention features a trimeric immunogen including a hyperglycosylated and cysteine-stabilized GCN4 transcriptional activator domain and three linked receptor binding domains of a Coronavirus spike protein, wherein each receptor binding domain includes a heterologous receptor binding motif of a Coronavirus, and wherein each receptor binding domain includes at least 1, 2, 3, or 4 or more non-native, N-linked, glycosylation sites. In preferred embodiments, the receptor binding domain includes 4 non-native, N-linked, glycosylation sites. In other embodiments, the receptor binding domain includes 5, 6, 7, or 9 non-native, N-linked glycosylation sites.

In some embodiments, the receptor binding domain of a Coronavirus spike protein includes a SARS-CoV-2, SARS-1, WIV1, MERS, Rs_672, Rp_Shaanxi2011, Cp_Yunnan2011, BtRf-HeB2013, BtRf-SX2013, BtRs-HuB2013, BtRs-GX2013, BtRs-YN2013, Longquan-140, YNLF_31C, YNLF_34C, As6526, Rs4081, Rs4237, Rs4247, Rs4255, BtRI_SC2018, BtRs_YN2018A, BtRS_YN2018C, BtRs_YN2018D, HKU9, HKU3-1, HKU4, HKU5, RaTG13, or SHC014 receptor binding domain.

In some embodiments, the heterologous receptor binding motif of a Coronavirus is the ACE2 binding motif of SARS-CoV-2 or a variant thereof.

In some embodiments, the receptor binding domain of a Coronavirus spike protein includes a receptor binding domain selected from a SARS-1 or WIV1 receptor binding domain and the heterologous receptor binding motif of a Coronavirus is the ACE2 receptor binding motif of SARS-CoV-2.

In another aspect, the invention features a receptor binding domain including ferritin. In some embodiments, the receptor binding domain is resurfaced. In some embodiments, the receptor binding domain includes rsWIV1 or rsSARS-1. In some embodiments, the receptor binding domain is conjugated to ferritin.

In yet another aspect, the invention features a ferritin nanoparticle including an immunogen including at least one receptor binding domain of a Coronavirus spike protein, wherein the receptor binding domain includes at least 2 non-native, N-linked, glycosylation sites. In preferred embodiments, the immunogen is monomeric, dimeric, or trimer. In some embodiments, the nanoparticle includes at least 3, 6, 9, or 12 receptor binding domains. In some embodiments, the receptor binding domain includes rsWIV1. In some embodiments, the receptor binding domain includes rsSARS-1. In some embodiments, the receptor binding domain is conjugated to ferritin.

In another aspect, the invention features a method of preventing a coronavirus infection in a subject or reducing the severity thereof, the method including administering to the subject an effective amount of the ferritin nanoparticle of the foregoing aspect or the receptor binding domain composition of the foregoing aspects, and a pharmaceutically acceptable carrier, thereby preventing the coronavirus infection in the subject or reducing the severity thereof.

In still another aspect, the invention features a method of preventing a coronavirus infection in a subject or reducing the severity thereof, the method including administering to the subject an effective amount of an aforementioned monomeric immunogen, dimeric immunogen, or the trimeric immunogen or a combination thereof of the monomeric, dimeric, and trimeric immunogens, and a pharmaceutically acceptable carrier, thereby preventing the coronavirus infection in the subject (e.g., a human) or reducing the severity thereof.

In some embodiments, the subject has previously been infected with SARS-CoV-2.

In some embodiments, the subject has previously been vaccinated against Covid-19.

In some embodiments, the subject has antibodies against SARS-CoV-2.

In some embodiments, two or more dimeric immunogens are administered to the subject. In some embodiments, the dimeric immunogens are the same. In some embodiments, the dimeric immunogens are different.

In some embodiments, two or more trimeric immunogens are administered to the subject. In some embodiments, the trimeric immunogens are the same. In some embodiments, three different trimeric immunogens are administered to the subject.

In still another aspect, the invention features any one of the following polypeptides including:

(SEQ ID NO: 11)
RVQPTESIVRFPNITNLCPFGEVFNATRFASVYAWNRSRISNCVA
DYSVLYNSSSFSTFKCYGVNATKLNDLCFTNVYADSFVIRGDEVR
QIAPGQTGKIADYNYKLPDDFTGCVIAWNSNNNDSKVGGNYSYLY
RLFRKSNLKPFERDNSTEIYQNGSTPCNGVEGFNCYFPLQNYSFQ
PTNGVGYQPYRVVVLSFELNHSPATVCGPKKGAGSSGSGRMKQIE
DKIENITSKIYNITNEIARIKKCCGNRT;

(SEQ ID NO: 12)
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYLY
RLFRKSNLKPFERDISTEIYQNGSTPCNGVEGFNCYFPLQSYGFQ
PTNGTGYQPYRVVVLSFELLHAPATVCGPKKGAGSSGSGRMKQIE
DKIENITSKIYNITNEIARIKKCCGNRT;

(SEQ ID NO: 13)
RVQPTESIVRFPNITNLCPFGEVFNATRFASVYAWNRSRISNCVA
DYSVLYNSSSFSTFKCYGVNATKLNDLCFTNVYADSFVIRGDEVR
QIAPGQTGKIADYNYKLPDDFTGCVIAWNSNNNDSKVGGNYNYLY
RLFRKSNLKPFERDNSTEIYQAGSTPCNGVEGFNCYFPLQSYGFQ
PTNGVGYQPYRVVVLSFELNHSPATVCGPKKGAGSSGSGRMKQIE
DKIENITSKIYNITNEIARIKKCCGNRT;

(SEQ ID NO: 14)
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYSYLY
RLFRKSNLKPFERDISTEIYQNGSTPCNGVEGFNCYFPLQNYSFQ
PTNGTGYQPYRVVVLSFELLHAPATVCGPKKGAGSSGSGRMKQIE
DKIENITSKIYNITNEIARIKKCCGNRT;

(SEQ ID NO: 15)
RVAPSKEVVRFPNITNLCPFWEVFNATTFPSVYAWNRSRISNCVA
DYSVLYNSTSFSTFACYGVNATALNDLCFSNVYADSFVVKGDDVR
QIAPGQTGVIADYNYKLPDDFRGCVLAWNSNNNDSKVGGNYNYLY
RLFRKSNLKPFERDNSTEIYQAGSTPCNGVEGFNCYFPLQSYGFQ
PTNGVGYQPYRVVVLSFELNNSPATVCGPKLGAGSSGSGRMKQIE
DKIENITSKIYNITNEIARIKKCCGNRT;
or

(SEQ ID NO: 16)
RVVPSGDVVRFPNITNLCDFGEVFNATKFPSVYAWNRSKISNCVADY
SVLYNSTFFSTFACYRVNATKLNDLCFSNVYADSFVVKGDDVRQIAP
GQTGVIADYNYKLPDDFKGCVLAWNSNNNDSKVGGNYNYLYRLFRKS
NLKPFERDNSTEIYQAGSTPCNGVEGFNCYFPLQSYGFQPTNGVGYQ
PYRVVVLSFELNNSPATVCGPKLGAGSSGSGRMKQIEDKIENITSKI
YNITNEIARIKKCCGNRT.

In some embodiments, the polypeptide further includes SGGLEVLFQGPGSSHHHHHHHH (SEQ ID NO: 17).

In yet another aspect, the invention features a nucleic acid including an open reading frame that encodes an immunogen of any one of aforementioned monomeric, dimeric, or trimeric immunogen or any one of the aforementioned polypeptides, receptor binding domains, or ferritin nanoparticles. In some embodiments, the nucleic acid is a messenger RNA.

In some aspects, the invention includes a composition including any of the aforementioned nucleic acids (e.g., a mRNA). In some embodiments, the composition includes a lipid nanoparticle.

In yet another aspect, the invention features a method of preventing a coronavirus infection in a subject (e.g., a human) or reducing the severity thereof, the method including administering to the subject an effective amount of the composition including any of the aforementioned nucleic acids or such nucleic acids lipid nano particle formulations and a pharmaceutically acceptable carrier, thereby preventing the coronavirus infection in the subject or reducing the severity thereof. In some embodiments, the polypeptide is a dimer. In some embodiments, the polypeptide is SARS-2hg. In some embodiments, the SARS-2hg polypeptide is a trimer.

As used herein, the terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can include a protein's or peptide's sequence. Polypeptides include any peptide or protein including two or more amino acids joined to each other by peptide bonds. As used herein, these terms refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In some embodiments, the invention features a peptide, polypeptide, or protein having 70%, 75%, 80%, 85%, 90%, 91%, 92%, 93%, 94%, 95%, 96%, 97%, 98%, 99%, or even 100% sequence identity to any of the peptides, polypeptides, or proteins as well as nucleic acid molecules as disclosed herein.

As used herein, the term “percent identity” or “sequence identity” refers to percent (%) sequence identity with respect to a reference polynucleotide sequence or a reference polypeptide sequence following alignment by standard techniques. Alignment for purposes of determining percent polynucleotide sequence identity or percent polypeptide sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32 (5): 1792-1797, 2004). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul et al., (1990) J. Mol. Biol., 215:403-410). As an illustration, the percent sequence identity of a given polypeptide sequence, A, to, with, or against a given polypeptide sequence, B, (which can alternatively be phrased as a given polypeptide sequence, A that has a certain percent sequence identity to, with, or against a given polypeptide sequence, B) is calculated as follows:
100 multiplied by (the fraction X/Y)
   where X is the number of amino acids scored as identical matches by a sequence alignment program (e.g., BLAST) in that program's alignment of A and B, and where Y is the total number of amino acids in B.

Immunogenic compositions that contain an immunogenically effective amount of a polypeptide described herein or fragments thereof, are also provided herein, and can be used in generating antibodies.

For vaccine formulations, an immunogenically effective amount of a composition can be provided, alone or in combination with other compounds, with an adjuvant, for example, Freund's incomplete adjuvant or aluminum hydroxide or as is described herein. The compound may also be linked with a carrier molecule, such as bovine serum albumin or keyhole limpet hemocyanin to enhance immunogenicity.

A pharmaceutical composition including one or more immunogens (e.g., monomeric, dimeric, or trimeric) or mRNAs as described herein are administered by a variety of methods known in the art.

The compositions disclosed herein include, for example, a (at least one) RNA having an open reading frame (ORF) encoding an immunogen or polypeptide described herein. In some embodiments, the RNA is a messenger RNA (mRNA). In some embodiments, the RNA (e.g., mRNA) further comprises a 5′ UTR, 3′ UTR, a poly(A) tail and/or a 5′ cap analog.

It should also be understood that a vaccine disclosed herein may include any 5′ untranslated region (UTR) and/or any 3′ UTR.

Nucleic acids include a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a beta-D-ribo configuration, alpha-LNA having an alpha-L-ribo configuration (a diastereomer of LNA), 2′-amino-LNA having a 2′-amino functionalization, and 2′-amino-alpha-LNA having a 2′-amino functionalization), ethylene nucleic acids, cyclohexenyl nucleic acids and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.” Similarly, disclosure of a polypeptide provides corresponding information regarding nucleic acids encoding such polypeptide.

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further include additional elements, e.g., 5′ and 3′ UTRs, but that those elements, unlike the ORF, need not necessarily be present in an RNA polynucleotide disclosed herein.

The invention provides several advantages. For example, rather than generating an immune response targeting the full SARS-CoV-2 spike protein, immunogens described herein aim to boost immunity to the SARS-CoV-2 ACE-2 receptor binding motif, which is part of the receptor binding domain.

Other features and advantages of the invention will be apparent from the following description of the preferred embodiments thereof, and from the claims.

### BRIEF DESCRIPTION OF THE DRAWINGS

FIG. 1A-FIG. 1G show a biochemical validation of resurfaced and hyperglycosylated immunogens. FIG. 1A shows an SDS-Page gel analysis of the SARS-2 receptor binding motif (RBM) construct with HRV 3C-cleavable 8×His and streptavidin binding peptide (SBP) tags. FIG. 1B shows ACE2 cell binding assay results for the SARS-2 RBM construct at 1 μM. FIG. 1C shows that after grafting the SARS-2 RBM onto the SARS-1 and WIV1 RBDs, conformationally specific Fabs B38 and CR3022 were used to confirm that these epitopes remained intact with comparable affinity to wild-type (9.7*10-7 M for B38 Fab and SARS-2 RBD; 2.7*10-8 M for CR3022 Fab and SARS-1 RBD; 3.7*10-8 M for CR3022 Fab and WIV1 RBD). FAB2G sensors were used with immobilized fabs. rsSARS-1 and rsWIV1 were the analytes. Titrations with B38 Fab were performed at 10 μM, 5 μM, 1 μM, and 0.5 μM. Titrations with CR3022 Fab were performed at 10 M and 5 M. Vendor-supplied software was used to generate an apparent K_{D}, or an approximate K_{D} in the case of titrations with two runs. FIG. 1D shows that candidate glycans were tested individually in the context of the SARS-CoV-2 RBD. All glycans were designed to mask epitopes outside the RBM. Therefore, biochemical validation was performed by assessing binding to the RBM-directed conformationally specific antibody B38 via single-hit BLI using FAB2G sensors with the RBD of interest as the analyte at 10 μM. Binding to ACE2 was also assessed via an ACE2 cell binding assay with antigen concentrations at 1 μM. Binding was binned subjectively into three categories: minimal (red), substantially reduced (yellow), and roughly intact (green). FIG. 1E shows that a conformationally specific Fab B38 was used to assess continued accessibility of the SARS-2 RBM in both monomeric and trimeric hyperglycosylated constructs. FAB2G sensors were used with immobilized fabs; hyperglycosylated coronavirus proteins were the analytes. Titrations with B38 Fab were performed at 7.5 μM, 5 μM, 2.5 μM, and 1 μM (rsSARS-1^{hg} trimer, rsWIV1^{hg} trimer); 5 μM, 2.5 μM, 1 μM, and 0.5 μM (SARS-2^{hg} trimer, SARS-2^{hg} monomer); 10 μM, 7.5 μM, 5 μM, 3.75 μM, and 2.5 μM (rsWIV1^{hg} monomer); 10 μM, 7.5 μM, 5 μM, and 2.5 μM (rsSARS-1^{hg} monomer). Vendor-supplied software was used to generate an apparent K_{D}. FIG. 1F shows BLI with conformationally specific Fabs CR3022 and S309 at 10 μM was compared to loading controls for each of the hyperglycosylated monomers to confirm a lack of binding. FIG. 1G shows ACE2 cell binding assay results for resurfaced and hyperglycosylated constructs at 1 M.

FIG. 2A-FIG. 2C show resurfacing and hyperglycosylation approaches for immune focusing. FIG. 2A shows the design schematic for resurfacing SARS-1 (rsSARS-1) and WIV1 (rsWIV1) scaffolds with the SARS-2 receptor binding motif (RBM), followed by design schematic for hyperglycosylating SARS-2 (blue), rsSARS-1 (green) and rsWIV1 (purple) receptor binding domains (RBDs). Non-native engineered glycans and native glycans are modeled; native SARS-2 RBD glycan at position 331 is omitted in the schematic. Mutations in the WIV1 and SARS-1 RBDs are shown in red and italicized in the linear diagram. All images were created using PDB 6MOJ. FIG. 2B shows the design schematic for generating RBD trimers appended onto a cystine-stabilized (red stars) hyperglycosylated GCN4 tag (PDB: 6VSB). FIG. 2C shows a schematic of immunization cohorts. The Trimer, Trimer^{hg}, and Cocktail^{hg} cohorts each contained 10 mice, while the Trivalent and RBM^{hg} cohorts each contained 5 mice.

FIG. 3A-FIG. 3K show immunogen production and biochemical validation. FIGS. 3A and 3B show the design and expression of two different versions of the SARS-2 RBD with additional putative N-linked glycosylation sites (PNGs) engineered onto the RBM. One construct has glycosylation sites at positions 475 and 501, while the other construct has glycosylation sites at positions 475, 501, 448, and 494 The latter is the RBM^{hg} construct. (PDB: 6MOJ). FIG. 3C shows the presence of glycans at positions 475 and 501 alone is sufficient to abrogate ACE2 binding to the RBM. FIG. 3D shows the representative size exclusion trace with (*) marking the trimeric constructs. Fractions in this peak were pooled and used for immunizations. Quantity of Expi293 transfection is in parentheses next to each label. FIG. 3E shows an SDS-PAGE analysis of purified trimers following removal of the affinity purification tags under non-reducing (NR) and reducing (R) conditions. The engineered disulfide bond at the C-terminus of the hyperglycosylated GCN4 tag separated under reducing conditions. Panel includes monomeric RBDs run under reducing conditions for comparison. FIG. 3F shows protein yields for purified trimeric constructs in Expi293 cells. FIG. 3G shows conformationally specific Fabs CR3022 and/or B38 were used to verify that trimer affinity was comparable (or greater than, due to increased avidity) wild-type RBD affinity. Fabs were immobilized to FAB2G sensors, and coronavirus proteins were the analytes. Trimers were titrated at 1 μM, 750 nM, 500 nM, and 250 nM. Monomeric SARS-1 and WIV1 RBDs were titrated at 10 μM, 5 μM, 2.5 μM, and 1 μM. Monomeric SARS-2 RBD was titrated at 10 μM, 1 μM, 500 nM, and 100 nM with B38 Fab and at 10 UM, 5 μM, 750 nM, and 250 nM with CR3022 Fab. Apparent K_{D} was obtained by vendor-supplied software. FIG. 3H shows that SDA-Page gel analysis of purified and cleaved trimeric constructs under non-reducing (NR) and reducing (R) conditions, showing the dissociation of the disulfide bond in the hyperglycosylated GCN4 tag (hgGCN4^{cys}) under reducing conditions. FIG. 3I shows the representative size exclusion chromatography traces for trimeric constructs. The trimer peak is marked with “*”, and fractions from this peak were pooled for HRV 3C cleavage and use as immunogens. Quantity of Expi293 transfection is in parentheses next to each label. FIG. 3J shows the protein yields for purified trimeric hyperglycosylated constructs in Expi293 cells. FIG. 3K shows the strict amino acid conservation across the SARS-2 RBD (Genbank MN975262.1), SARS-1 RBD (Genbank ABD72970.1), and WIV1 RBD (Genbank AGZ48828.1) is depicted using dark blue on the structure for matches between all three genes, light blue for matches between two genes, and silver for positions where all genes differ (PDB: 6MOJ).

FIG. 4A-FIG. 4D show assessment of SARS-2 RBD immune focusing via serum analysis from cohorts. FIG. 4A shows serum following immunizations was assayed in ELISA at day 35 with wild-type SARS-2 RBD and RBM^{hg}. Statistical significance was determined using the Mann-Whitney U test (*=p<0.05, **=p<0.01). FIG. 4B depicts day 35 serum samples from the Trimer^{hg} and Cocktail^{hg} cohorts showing significantly less binding to the SARS-1 and WIV1 RBDs compared to the SARS-2 RBD (see FIGS. 5B-5C). However, when assayed against rsSARS-1 and rsWIV1 RBDs, these sera no longer show statistically significant differences in binding compared to SARS-2 RBD as determined using the Kruskal-Wallis test with post-hoc analysis using Dunn's test corrected for multiple comparisons. FIG. 4C shows the approximate locations of representative antibody epitopes from each of the four SARS-2 RBD-directed antibody classes (Barnes et al., Nature 588, 682-687 (2020)). (PDB: 6MOJ). FIG. 4D shows the percent competition in ELISAs using day 35 mouse sera in the presence of competing IgGs vs. a no-IgG control. SARS-2 RBD was the coating antigen. Statistical significance was determined using the Kruskal-Wallis test with post-hoc analysis using Dunn's test corrected for multiple comparisons (*=p<0.05, **=p<0.01, ***=p<0.001); ns=not significant.

FIG. 5A-FIG. 5H show serum antigenicity assessed via ELISA. Serum following immunizations from the Trimer (FIG. 5A), Trimer^{hg} (FIG. 5B), Cocktail^{hg} (FIG. 5C), RBM^{hg} (FIG. 5D), and Trivalent (FIG. 5E) cohorts was assayed in ELISA at day 35 with different coronavirus antigens. Statistical significance was determined using Kruskal-Wallis test with post-hoc analysis using Dunn's test corrected for multiple comparisons (*=p<0.05, **=p<0.01, ***=p<0.001, ****=p<0.0001). FIG. 5F shows a serum ELISA was performed for Trivalent cohort with an irrelevant protein (influenza hemagglutinin head) tagged with the hyperglycosylated GCN4 tag (HA-GCN4) to measure tag-directed antibody responses. The Trivalent cohort was selected because it had the highest serum ELISA titers. For the coating HA-GCN4 protein, a purification size exclusion trace (fractions in the peak marked with “*” were pooled) and an SDS-PAGE gel run under non-reducing (NR) and reducing (R) conditions are shown. FIG. 5G shows serum ELISAs performed against the relevant hyperglycosylated immunogens for the Trimer^{hg} and Cocktail^{hg} cohorts. There was no statistically significant difference in endpoint titers within the Trimer^{hg} or Cocktail^{hg} cohorts across these coating antigens, as determined by the Mann-Whitney U test and the Kruskal-Wallis test, respectively. FIG. 5H shows for all cohorts, day 35 serum samples were used in ELISAs to assess binding to RaTG13 and SHC014 RBDs.

FIG. 6A-FIG. 6E show the potency and characterization of SARS-like coronavirus neutralization response. FIG. 6A shows that day 35 serum from all mice was assayed for neutralization against SARS-2, RaTG13, SARS-1, WIV1, and SHC014 pseudoviruses (arranged in order of genetic similarity of the full-length spike to SARS-2). Neutralization potency was computed using scaled endpoint serum ELISA titers. Statistical significance was determined using the Kruskal-Wallis test with post-hoc analysis using Dunn's test corrected for multiple comparisons (*=p<0.05, **=p<0.01, ***=p<0.001, ****=p<0.0001, ns=not significant). FIG. 6B shows the approximate locations of representative antibody epitopes from the two non-RBM-directed SARS-2 RBD-directed antibody classes (Barnes et al., Nature 588, 682-687, 2020) and ADG-2-like antibodies on the WIV1 RBD. (PDB: 6MOJ). FIGS. 6C-6D show antibody competition ELISAs with WIV1 RBD as the coating antigen. Bars show the mean percent binding lost, with error bars representing the standard error of the mean. Comparisons were performed using the Kruskal-Wallis test with post-hoc analysis using Dunn's test corrected for multiple comparisons (*=p<0.05, ** p<0.01, ***=p<0.001, ****=p<0.0001). FIG. 6E shows day 35 serum assayed against SARS-2 variant pseudoviruses for neutralization. Statistical significance was determined using the Kruskal-Wallis test with post-hoc analysis using Dunn's test corrected for multiple comparisons (*=p<0.05, **=p<0.01).

FIG. 7A-FIG. 7E show neutralization against related sarbecoviruses and SARS-2 variants of concern. FIG. 7A shows day 35 serum from all mice was assayed for neutralization against SARS-2, RaTG13, SARS-1, WIV1, and SHC014 pseudoviruses. Statistical significance was determined using the Kruskal-Wallis test with post-hoc analysis using Dunn's test corrected for multiple comparisons (*=p<0.05, **=p<0.01, ***=p<0.001). FIG. 7B shows pseudovirus neutralization assays were used to calculate NT50 values for SARS-2, SARS-1, WIV1, RaTG13, and SHC014 from all cohorts. All NT50s are from day 35 sera. Statistical significance was determined using the Kruskal-Wallis test with post-hoc analysis using Dunn's test corrected for multiple comparisons in the case of a significant Kruskal-Wallis test (*=p<0.05). FIG. 7C shows the structural depiction of SARS-2 variant RBD mutations for B.1.351 (red), as well as ACE2 contact residues (cyan). (PDB: 6MOJ) Sequences depict all spike mutations across select variants. FIG. 7D shows day 35 serum assayed in ELISA against SARS-2 RBD (WT) and SARS-2 RBD with K417N, E484K, and N501Y mutations (B.1.351). Statistical significance was determined using the Wilcoxon sign rank test (*=p<0.05, **=p<0.01; ns=not significant). FIG. 7E shows that the pseudovirus neutralization assays were used to calculate NT50 values for P.1, B.1.1.7, and B.1.351 from all cohorts. All NT50s are from day 35 sera. Statistical significance was determined using the Kruskal-Wallis test; no differences are statistically significant.

FIGS. 8A-FIG. 8E show that the immune response following boosting with ferritin nanoparticle multimerization of SARS-2^{hg}. FIG. 8A shows the design schematic of a multimerized version of SARS-2^{hg} using SpyTag-SpyCatcher conjugation to a ferritin nanoparticle. FIG. 8B shows that the serum following immunization was assayed in ELISA at day 35 with wild-type SARS-2 RBD and RBM^{hg}. Statistical significance was determined using Mann-Whitney U test (*=p<0.05). FIG. 8C shows that the day 35 serum titers to wild-type SARS-2 RBD were also compared to titers against SARS-2^{hg} and the unconjugated ferritin nanoparticle-SpyCatcher fusion. A Kruskal-Wallis test was performed and detected no significant differences between the serum antibody responses to these three proteins (ns=not significant). FIG. 8D shows that the day 35 serum was assayed for neutralization against SARS-2, SARS-1, and WIV1 pseudoviruses. Statistical significance was determined using the Kruskal-Wallis test with post-hoc analysis using Dunn's test corrected for multiple comparisons (*=p<0.05, **=p<0.01, ***=p<0.001). FIG. 8E shows that the day 35 serum was assayed against SARS-2 variant pseudoviruses for neutralization. Statistical significance was determined using the Kruskal-Wallis test with post-hoc analysis using Dunn's test corrected for multiple comparisons (*=p<0.05).

FIG. 9A-FIG. 9E show the (related to FIG. 8) serum antigenicity for the Nanoparticle cohort. FIG. 9A shows that the serum following immunization was assayed in ELISA at day 35 with different coronavirus antigens. Statistical significance was determined using the Kruskal-Wallis test with post-hoc analysis using Dunn's test corrected for multiple comparisons (*=p<0.05, **=p<0.01, ***=p<0.001). FIG. 9B shows the comparison of day 35 SARS-2 RBD ELISA endpoint titers in the Nanoparticle and Trimer^{hg} cohorts. Statistical significance was determined using the Mann-Whitney U test (ns=not significant). FIG. 9C shows day 35 serum samples assayed against rsSARS-1 and rsWIV1 RBDs no longer show statistically significant differences in binding compared to SARS-2 RBD as determined using the Kruskal-Wallis test with post-hoc analysis using Dunn's test corrected for multiple comparisons. FIG. 9D shows the comparison of day 35 SARS-2 pseudovirus neutralization in the Nanoparticle and Trimer^{hg} cohorts. Statistical significance was determined using the Mann-Whitney U test (ns=not significant). FIG. 9E shows day 35 serum assayed in ELISA against SARS-2 RBD (WT) and SARS-2 RBD with K417N, E484K, and N501Y mutations (B.1.351). Statistical significance was determined using the Wilcoxon sign rank test (*=p<0.05, **=p<0.01).

FIG. 10A-FIG. 10J show that SARS-2 RBD-directed B cell characteristics. FIG. 10A shows the gating scheme for isolating IgG+ B cells that are SARS-2 spike double-positive and SARS-CoV-2 RBD positive. Spleens were harvested at day 42 and SARS-2 RBD-directed IgG+ B cells were isolated via flow cytometry and sequenced. B cell receptor sequencing was used to characterize (FIG. 10B) heavy and (FIG. 10C) light chain V-gene usage. All gene families listed are *01 except VH1-84*02. Complementarity determining region 3 (CDR3) length (FIG. 10D) and percent somatic hypermutation (SHM) (FIG. 10E) were also analyzed for each sequence. SHM was not analyzed for cohorts with uncertain IMGT V-gene assignments. Statistical significance was determined using the Kruskal-Wallis test with post-hoc analysis using Dunn's test corrected for multiple comparisons (*=p<0.05, **=p<0.01). Within each mouse, clonal lineages were analyzed on the basis of CDRH3 similarity. CDRH3 groupings are represented by count (FIG. 10F) and proportion (FIG. 10G) within each mouse. Not all sequences were successfully grouped into lineages. FIG. 10H shows the phylogenetic tree of a large clonal lineage containing Ab16 and Ab17 generated using Cloanalyst to infer common ancestors. FIG. 10I shows that BLI was performed using Fabs representative of lineages expanded in RBM-focusing cohorts. Ni-NTA biosensors were used with Fabs bound to the sensor using the 8×His tag and RBDs in solution as the analyte. Titrations were performed at 10 μM, 5 μM, 2.5 μM, and 1 μM. Vendor-supplied software was used to generate an apparent K_{D}. FIG. 10J shows the conformationally specific Fabs ADI-55688, ADI-55689, and ADI-56046, which target a conserved RBM epitope, were used to assess binding to the RBM^{hg}, SARS-2^{hg}, rsSARS-1^{hg}, and rsWIV1^{hg} monomers via BLI. FAB2G sensors were used with immobilized fabs; coronavirus proteins were the analytes. Titrations were performed at 10 μM, 5 μM, 2.5 μM, and 1.25 μM (SARS-2 RBD and SARS-2^{hg} with ADI-55689, SARS-2 RBD and SARS-2^{hg} with ADI-56046, all titrations with RBM^{hg}); 3 μM, 1.5 μM, 0.75 UM, and 0.375 μM (rsSARS-1^{hg} and rsWIV1^{hg} with ADI-56046); 10 μM, 5 μM, 2.5 μM, and 1 μM (all titrations with ADI-55688 except RBM^{hg}). Minimal binding was detected to ADI-55689 Fab with rsSARS-1^{hg} and rsWIV1^{hg}. Vendor-supplied software was used to generate an apparent K_{D}.

FIG. 11A-FIG. 11B show SARS-2 RBD-directed B cell characteristics. Spleens were harvested at day 42 and SARS-2 RBD-directed IgG+ B cells were isolated via flow cytometry and sequenced. FIG. 11A shows antibodies representative of lineages that were expanded in RBM-focusing cohorts were expressed recombinantly as Fabs, and their binding was characterized via BLI. FIG. 11B shows pseudovirus neutralization for these antibodies was also characterized.

FIG. 12A-FIG. 12F show structural characterization of antibodies from RBM-focused immune response. FIG. 12A shows a low resolution cryoEM map with model of Ab20 as Fab (pink) bound to RBD (blue) with the RBM (gray) shown (RBD is from PDB 6VXX); for ease of viewing only a single RBD and Fab are shown. FIG. 12B shows a low resolution cryoEM map with model of Ab16 as Fab (pink) bound to RBD (blue) with the RBM (gray) shown (RBD is from PDB 7DX9); for ease of viewing only a single RBD and Fab are shown. (FIG. 12C) Model from (FIG. 12B) with docked ACE2 (from PDB 6MOJ). FIG. 12D shows a CryoEM map with 3 docked RBDs (blue, with RBMs in grey) from the “3 RBD up” spike in PDB 7DX9. RBDs are shown in ribbon and the Fab Ab16 removed to show its density and the slight outward rotation of the RBD required to better fit the density compared to the docked model. FIG. 12E shows the interface of co-crystal structure of SARS-2 RBD and Ab17 heavy (yellow) and light (purple) chain complex. FIG. 12F shows the surface of the SARS-2 RBD in contact with Ab17 heavy (yellow) and light (purple) chain residues. (Related to FIG. 14)

FIG. 13A-FIG. 13E show the SARS-2 protection studies in K18-hACE2 transgenic mice. FIG. 13A is a schematic showing passive transfer and SARS-2 D614G live virus challenge timeline. FIG. 13B shows that following inoculation with SARS-2 D614G, each mouse was weighed daily. There were 5 mice in each of the 3 cohorts. The mean and standard error of the mean for the three cohorts are shown at each time point. Cohorts were compared using an ordinary one-way ANOVA with Dunnett's test of area under the curve (*=p<0.05, **=p<0.01, ns=not significant). At 6 days post-inoculation, tissues were harvested and viral RNA levels in the lungs (FIG. 13C), heart (FIG. 13D), and nasal wash (FIG. 13E) were assessed by RT-qPCR RNA. Cohorts were compared using a Kruskal Wallis test with post-hoc analysis using Dunn's test corrected for multiple comparisons (*=p<0.05, **=p<0.01, ns=not significant).

FIG. 14A-FIG. 14B show supporting data for moderate-resolution structures of Ab16 and Ab20 complexes. Fourier shell correlation plots show the nominal resolution of the spike complex of Ab16 is 5.5 Å (FIG. 14A) and Ab20 is 9.2 Å (FIG. 14B) (0.143 cutoff).

FIG. 15A-FIG. 15D show the resurfacing and hyperglycosylation approaches for immune focusing. FIG. 15A shows the design schematic for resurfacing SARS-1 (rsSARS-1) and WIV1 (rsWIV1) with the SARS-2 receptor binding motif (RBM). Design schematic for hyperglycosylating SARS-2 (FIG. 15B), rsSARS-1 (FIG. 15C) and rsWIV1 (FIG. 15D) receptor binding domains (RBDs). Non-native engineered glycans and native glycans are modeled; native SARS-2 RBM glycan at position 331 is omitted in the schematic. Mutations in the WIV1 and SARS-1 RBDs are shown in red and italicized in the linear diagram. All images were created using PDB 6MOJ.

FIG. 16A-FIG. 16D show a serum analysis from cohorts. FIG. 16A is a schematic of immunization cohorts; N=number of mice in each cohort (FIGS. 16B-16C) Serum following immunizations was assayed in ELISA at day 56 with different coronavirus antigens. Statistical significance was determined using Kruskal-Wallis test with post-hoc analysis using Dunn's test corrected for multiple comparisons or Mann-Whitney U test (*=p<0.05, **=p<0.01, ***=p<0.001, ****=p<0.0001). FIG. 16D shows that the day 56 serum samples assayed against rsSARS-1 and rsWIV1 RBDs no longer show statistically significant differences in binding compared to SARS-2 RBD as determined using Kruskal-Wallis test with post-hoc analysis using Dunn's test corrected for multiple comparisons.

FIG. 17A-FIG. 17E show the potency and characterization of SARS-like coronavirus neutralization response. FIG. 17A shows day 56 serum from all mice was assayed for neutralization against SARS-2, RaTG13, SARS-1, WIV1, and SHC014 pseudoviruses (arranged in order of genetic similarity of the full-length spike to SARS-2). Neutralization potency was computed using scaled endpoint serum ELISA titers. Statistical significance was determined using the Kruskal-Wallis test with post-hoc analysis using Dunn's test corrected for multiple comparisons (*=p<0.05, **=p<0.01, ***=p<0.001, ns=not significant). FIG. 17B shows day 56 serum from all mice was assayed for neutralization against SARS-2, RaTG13, SARS-1, WIV1, and SHC014 pseudoviruses. Statistical significance was determined using the Kruskal-Wallis test with post-hoc analysis using Dunn's test corrected for multiple comparisons (*=p<0.05, **=p<0.01, ***=p<0.001). FIG. 17C shows approximate locations of representative antibody epitopes from each of the four SARS-2 RBD-directed antibody classes (21) and ADG-2-like antibodies on the SARS-2 RBD. (PDB: 6MOJ) FIG. 17D shows antibody competition ELISAs with WIV1 RBD as the coating antigen. Bars show the mean percent biding lost, with error bars representing the standard error of the mean. Comparisons were performed using the Kruskal-Wallis test with post-hoc analysis using Dunn's test corrected for multiple comparisons (*=p<0.05, ** p<0.01, ***=p<0.001, ****=p<0.0001). FIG. 17E shows day 56 serum was assayed against SARS-2 variant pseudoviruses for neutralization. Statistical significance was determined using the Kruskal-Wallis test with post-hoc analysis using Dunn's test corrected for multiple comparisons (*=p<0.05, **=p<0.01).

FIG. 18A-FIG. 18G show the SARS-2 RBD-directed B cell characteristics. Spleens were harvested at day 63 and SARS-2 RBD-directed IgG+ B cells were isolated via flow cytometry. B cell receptor sequencing was used to characterize (FIG. 18A) heavy and light chain V-gene usage. All gene families listed are *01 except VH1-84*02. Complementarity determining region 3 (CDR3) length (FIG. 18B) and percent somatic hypermutation (SHM) (FIG. 18C) were also analyzed for each sequence. SHM was not analyzed for cohorts with uncertain IMGT V-gene assignments. Statistical significance was determined using the Kruskal-Wallis test with post-hoc analysis using Dunn's test corrected for multiple comparisons (*=p<0.05, **=p<0.01). FIG. 18D shows antibodies representative of lineages that were expanded in RBM-focusing cohorts were expressed recombinantly as Fabs, and their binding was characterized via BLI (FIG. 18E) low resolution cryoEM map with model of Ab16 as Fab (blue) bound to RBD (gray) with the RBM (magenta) shown (RBD is from PDB 7DX9); for ease of viewing only a single RBD and Fab are shown. FIG. 18F is a model from (FIG. 18E) with docked ACE-2 (from PDB 6MOJ) cryoEM map (FIG. 18G) with 3 RBDs (gray) in ribbon and the Fab Ab16 removed to show its density and the slight outward rotation of the RBD required to better fit the density compared to the docked 3 RBD up conformation from PDB 7DX9.

FIG. 19A-FIG. 19D show serum ELISAs with coronavirus proteins. Serum ELISAs were performed using day 56 serum samples to assess binding to coronavirus proteins. These assays were performed on the Trimer^{hg} and Cocktail^{hg} cohorts (FIG. 19A) and the ARBM and Trimer cohorts (FIG. 19B). Endpoint titers were calculated based on curves fit using a sigmoidal model. FIG. 19C shows that binding was also assayed against the rsSARS-1 and rsWIV1 RBDs using sera from the Trimer^{hg} and Cocktail^{hg} cohorts. FIG. 19D shows that for all cohorts, day 56 serum samples were used in ELISAs to assess binding to RaTG13 and SHC014 RBDs.

FIG. 20A-FIG. 20C show serum ELISAs with hyperglycosylated immunogens. Serum ELISAs were performed against the relevant hyperglycosylated immunogens for the Trimer^{hg} and Cocktail^{hg} cohorts. FIG. 20A shows that endpoint titers against all hyperglycosylated immunogens do not vary significantly from titers against wildtype SARS-2 RBD. Endpoint titers were calculated using curves fit with a sigmoidal model to data from the (FIG. 20B) Trimer^{hg} and (FIG. 20C) Cocktail^{hg} cohorts. There was no statistically significant difference in binding within the Trimer^{hg} or Cocktail^{hg} cohorts across these coating antigens, as determined by the Mann-Whitney U test and the Kruskal-Wallis test, respectively.

FIG. 21 shows serum neutralization titers organized by cohort. Pseudovirus neutralization assays were used to calculate NT50 values for SARS-2, SARS-1, WIV1, RaTG13, and SHC014 from all cohorts. All NT50s are from day 56 sera. Statistical significance was determined using the Friedman test to account for sample pairing with post-hoc analysis using Dunn's test corrected for multiple comparisons (*=p<0.05, **=p<0.01).

FIG. 22 shows serum neutralization titers organized by pseudovirus. Pseudovirus neutralization assays were used to calculate NT50 values for SARS-2, SARS-1, WIV1, RaTG13, and SHC014 from all cohorts. All NT50s are from day 56 sera. Statistical significance was determined using the Kruskal-Wallis test with post-hoc analysis using Dunn's test corrected for multiple comparisons; no differences are statistically significant.

FIG. 23A-FIG. 23C show the binding of SARS-2 variants. FIG. 23A is a structural depiction of SARS-2 variant RBD mutations for 501Y.V2 (red), as well as ACE2 contact residues (cyan). (PDB: 6MOJ) Sequences depict all spike mutations across select variants. FIG. 23B shows that the day 56 serum was assayed in ELISA against SARS-2 RBD (WT) and SARS-2 RBD with K417N, E484K, and N501Y mutations (B.1.351). Statistical significance was determined using the Wilcoxon sign rank test (*=p<0.05, **=p<0.01; ns=not significant). FIG. 23C shows that endpoint titers were calculated based on curves fit to ELISA data using a sigmoidal model.

FIG. 24 shows the 2D class averages of the SARS-CoV-2 Spike: Ab16 complex. Selected 2D class averages generated for the SARS-CoV-2 spike in complex with Ab16 Fab.

FIG. 25A-FIG. 25D portray the characterization of resurfaced receptor binding domain (rsRBD) nanoparticle immunogens. FIG. 25A shows a cartoon depiction of the rsSARS-1 and rsWIV1 RBDs. The grafted SARS-2 RBM is indicated by the small grey circles, while the concentric purple (WIV1) and green (SARS-1) circles indicate the RBD scaffolds. FIG. 25B shows a schematic of the conjugated rsSARS-1 and rsWIV1 nanoparticles. FIG. 25C shows a representative size exclusion trace showing purification traces for the SpyCatcher nanoparticle (grey), rsSARS-1 (green), and rsWIV1. FIG. 25D shows a representative size exclusion trace showing the conjugated rsSARS-1 nanoparticle (green) and rsWIV1 nanoparticle (purple) in the first peak at ˜8 mL. The second peak in both traces at ˜15 mL represents excess RBD from the conjugation reaction.

FIG. 26 is a schematic of immunization regimens for two cohorts of n=5 mice each receiving a homologous prime-boost of a single conjugated nanoparticle.

FIG. 27A-FIG. 27D depict the characterization of serum reactivity. Day 35 sera collected from (FIG. 27A) the rsSARS-1 cohort and (FIG. 27B) the rsWIV1 cohort were assayed against selected coronavirus proteins. Statistical significance was determined using Kruskal-Wallis test with post-hoc analysis using Dunn's test corrected for multiple comparisons (*=p<0.05, **=p<0.01); non-significant comparisons are not marked. FIG. 27C shows extended serum reactivity was measured against coronavirus RBDs that were not included in either immunogen. FIG. 27D shows serum titers were also measured against the unconjugated SpyCatcher nanoparticle. Statistical significance was determined using the Mann-Whitney U test; ns=not significant.

FIGS. 28A and 28B depict the results of pseudovirus neutralization. Pseudovirus neutralization of day 35 sera collected from (FIG. 28A) the rsSARS-1 cohort and (FIG. 28B) the rsWIV1 cohort was assayed against SARS-2, SARS-1, and WIV1. Statistical significance was determined using Kruskal-Wallis test with post-hoc analysis using Dunn's test corrected for multiple comparisons (*=p<0.05, **=p<0.01); non-significant comparisons are not marked.

FIG. 29A-FIG. 29E depict the design, expression, and in vivo characterization of a hyperglycosylated class 4-focusing immunogen. FIG. 29A shows a schematic for hyperglycosylated class 4-focusing immunogen; both non- and native engineered glycans are included. FIG. 29B shows a schematic of immunization cohorts; each cohort contains N=15 total mice. Two separate replicates of this experiment were performed, the first with N=5 mice and the second with N=10 mice. FIG. 29C shows Ab16 (teal), CR3022 (violet) epitopes and overlap (yellow) shown on SARS-2 RBD (PDB: 6MOJ). FIG. 29D shows day 35 serum competition with Ab16 assayed in ELISA coated with SARS-2 RBD. FIG. 29E shows day 35 serum assayed against wild-type SARS-2 SARS-2 variants, and related sarbecoviruses for neutralization. For (D, E), bars represent mean+/−SEM. Statistical significance determined using the Mann-Whitney U test (*=p<0.05, **=p<0.01, ***=p<0.001, ****=p<0.0001, ns=not significant).

FIG. 30A-FIG. 30J depict hyperglycosylated (HG) immunogen characterization in vitro and in vivo. FIG. 30A shows an ELISA binding of Ab16 to both the monomeric HG immunogen and wild-type SARS-2 RBD, and endpoint titers were compared. FIG. 30B shows that BLI was performed with conformationally specific Fabs representing the four Barnes classes: B38 (class 1), P2B-2F6 (class 2), S309 (class 3), and CR3022 (class 4) loaded onto FAB2G sensors. The monomeric HG immunogen was the analyte at 10 μM. All Fabs showed no evidence of binding to the HG immunogen. Furthermore, the resultant traces were well below the no-Fab loading control for the HG immunogen, which confirms a lack of binding. FIG. 30C shows a SDS-PAGE analysis of trimeric hyperglycosylated RBD immunogen under non-reducing (NR) and reducing (R) conditions; lanes containing unrelated samples have been removed.

FIG. 30D shows representative size exclusion traces with (*) marking the trimeric constructs for the SARS-2 wild-type RBD trimer (WT) and the HG immunogen (HG). Fractions in these peaks were pooled for purification tag cleavage and use in immunizations. FIG. 30E shows stability testing of the WT and HG immunogen under a variety of condition shows a minimal loss in binding to conformationally specific monoclonal antibody Ab16 as compared to a control sample, with no significant difference in Ab16 binding between the two immunogens (Mann Whitney U test; ns=not significant). FIG. 30F shows an antibody competition with Ab16 (a class 4 antibody) was assessed via BLI. SARS-2 RBD at 8 μM was complexed with at least a 5-molar excess Ab16 Fab for 30 minutes, and then binding of the complex to the B3E3, B8E8, and D2G2 Fabs was measured via BLI and compared to binding of SARS-2 RBD at 8 μM without Ab16. FAB2G sensors were used with B3E3, B8E8, or D2G2 immobilized and SARS-2 RBD: Ab16 complexes or SARS-2 RBD as the analytes. Day 35 sera from the (FIG. 30G) WT and (FIG. 30H) HG cohorts were assayed for binding to multiple coronavirus proteins by ELISA. Bars represent mean+/−SEM. “Spike” refers to the double-proline stabilized SARS-2 spike used in the prime immunization; all other coating antigens are sarbecovirus RBDs. Statistical significance was determined using Kruskal-Wallis test with post-hoc analysis using Dunn's test corrected for multiple comparisons, and pairwise comparisons without pictured bars were not significant (*=p<0.05, **=p<0.01, ***=p<0.001, ****=p<0.0001). Day 35 serum from the (FIG. 30I) WT and (FIG. 30J) HG cohorts was assayed against sarbecoviruses for neutralization. Bars represent mean+/−SEM. Statistical significance determined using the Kruskal-Wallis test with post-hoc analysis using Dunn's test corrected for multiple comparisons, and pairwise comparisons without pictured bars were not significant (*=p<0.05, **=p<0.01, ***=p<0.001, ****=p<0.0001).

FIG. 31 is a graphical abstract of Example IV. Biological samples were obtained from human donors with preexisting immunity to SARS-CoV-2. Human antibodies elicited by SARS-2 vaccination or infection that bind broadly to different sarbecovirus receptor binding domains were characterized. Broadly cross-neutralizing antibodies target two conserved epitopes. Subsequent structure-guided immunogen design focuses murine antibody responses to one such epitope in the context of preexisting immunity, conferring improved Omicron neutralization.

FIG. 32A-FIG. 32D illustrate heterotrimeric immunogen design and expression. FIG. 32A shows a schematic of heterotrimeric immunogen designs. FIG. 32B shows a representative size exclusion trace with (*) marking the trimeric constructs. Fractions in this peak were pooled and further purified.

FIG. 32C shows a SDS-PAGE analysis of purified trimers following removal of the affinity purification tags under non-reducing (NR) and reducing (R) conditions. FIG. 32D shows an antibody binding assessed (red=no binding, green=binding) via BLI with FAB2G sensors loaded with selected Fabs, with monomeric RBDs or heterotrimeric immunogens at 5 or 10 μM as analytes in solution.

FIG. 33A-33C depict the results of immunizations with heterotrimeric immunogens in a mouse model. FIG. 33A shows a schematic of immunization regimens. Two immunization cohorts (n=5 mice each) were primed with SARS-2 2-proline-stabilized spike protein on day 0 and then boosted with either the heterotrimer of wild-type RBDs (“WT Heterotrimer” cohort) or a heterotrimer of resurfaced RBDs (“rsHeterotrimer” cohort) at days 21 and 42. FIG. 33B shows serum collected at day 56 was assayed in ELISA against different coronavirus antigens, including (FIG. 33C) the wild-type SARS-2 RBD and the SARS-2 RBM^{hg} construct. Statistical significance was determined using Kruskal-Wallis test with post-hoc analysis using Dunn's test corrected for multiple comparisons (FIG. 33B) or the Mann Whitney U test (FIG. 33C) (*=p<0.05, **=p<0.01); ns=not significant.

FIG. 34A-FIG. 34B depict B cell and neutralization analysis. FIG. 34A shows that flow cytometry was used to bin SARS-2 spike-directed IgG+ B cell responses based on targeting of RBM, non-RBM RBD, and non-RBD spike “remainder” epitopes. Statistical significance was determined using Kruskal-Wallis test with post-hoc analysis using Dunn's test corrected for multiple comparisons (*=p<0.05, **=p<0.01); ns=not significant. FIG. 34B shows a pseudovirus neutralization was assayed against a panel of sarbecoviruses, ordered here based on spike amino acid sequence similarity to SARS-2. Statistical significance was determined using Kruskal-Wallis test with post-hoc analysis using Dunn's test corrected for multiple comparisons (*=p<0.05, **=p<0.01); non-significant comparisons not marked.

FIG. 35A-FIG. 35G portray resurfaced immunogen design and expression. FIG. 35A shows a design schematic of v1, v2, and v3 resurfaced constructs, depicting RBM grafts (grey) on heterologous coronavirus scaffolds (red). FIG. 35B shows a SDS-PAGE analysis of monomers under non-reducing conditions. FIG. 35C shows a representative size exclusion trace with (*) marking the monomeric constructs. Fractions in this peak were pooled for further characterization. FIG. 35D shows a RBM-directed Fab B38 was used to confirm conformational integrity of the SARS-2 RBM grafted onto rsYNLF-34C. FAB2G sensors were used with immobilized Fab to measure binding via BLI to rsYNLF-34C at 10 μM, 5 μM, 1 μM, and 0.5 M. Binding of CR3022 and S309 Fabs to rsYNLF-34C at 10 μM was also assayed, but no binding was detected. FIG. 35E shows a SDS-PAGE analysis of dimers under non-reducing conditions. FIG. 35F shows a representative size exclusion trace with (*) marking the dimeric constructs. Fractions in this peak were pooled for use as immunogens. FIG. 35G shows pairwise comparisons of amino acid sequence identity in the non-RBM portions of selected sarbecovirus RBDs.

FIG. 36A-FIG. 36C depict the results of antigenic distance investigation immunizations. FIG. 36A shows a schematic of immunization regimens. Two immunization cohorts (n=5 mice each) were primed with SARS-2 2-proline-stabilized spike protein on day 0 and then boosted with a cocktail of either the rsSARS-1 and rsWIV1 dimers (“AgDistlow” cohort) or the rsWIV1 and rsYNLF-34C dimers (“AgDisthigh” cohort) at days 21 and 42. FIG. 36B shows serum collected at day 56 was assayed in ELISA against different coronavirus antigens. FIG. 36C shows pseudovirus neutralization was assayed against a panel of sarbecoviruses, ordered here based on spike amino acid sequence similarity to SARS-2. Statistical significance for (FIGS. 36B, FIG. 36C) was determined using Kruskal-Wallis test with post-hoc analysis using Dunn's test corrected for multiple comparisons (*=p<0.05, **=p<0.01); non-significant comparisons not marked.

### DETAILED DESCRIPTION

I. Epitope Grafting of the SARS-CoV-2 Receptor Binding Motif onto Heterologous Coronavirus Scaffolds

Eliciting antibodies to surface-exposed viral glycoproteins can generate protective responses that control and prevent future infections. Targeting conserved sites may reduce the likelihood of viral escape and limit spread of related viruses with pandemic potential. As is presented below, we leveraged rational immunogen design to focus humoral responses on conserved epitopes. Using glycan engineering and epitope scaffolding, we directed murine serum antibody responses to conserved receptor binding motif (RBM) and domain (RBD) epitopes in the context of SARS-CoV-2 spike imprinting. Whereas all engineered immunogens elicited a robust SARS-CoV-2-neutralizing serum response, the RBM-focusing immunogens exhibited increased potency against related sarbecoviruses, SARS-CoV, WIV1-CoV, RaTG13-CoV, and SHC014-CoV; structural characterization of representative antibodies defined a conserved epitope. Furthermore, the RBM-focused sera conferred protection against SARS-CoV-2 challenge. Thus, RBM focusing is a promising strategy to elicit breadth across emerging sarbecoviruses without compromising SARS-CoV-2 protection. Broadly, these engineering strategies are adaptable to other viral glycoproteins for targeting conserved epitopes.

In particular, we designed protein-based immunogens that used hyperglycosylation of the RBD and a “resurfacing” approach that grafts the RBM from SARS-2 onto heterologous coronavirus-based RBD scaffolds. We leveraged these immunogens as tools to interrogate immune refocusing in the context of SARS-2 spike imprinting. We immunized mice that were primed with SARS-2 spike as a surrogate for pre-existing immunity imprinted by vaccination or natural infection. We found that boosting with different regimens containing our engineered immunogens could selectively focus serum responses to either the RBM or to non-RBM epitopes. Importantly, even the RBM-focused response targets broadly conserved epitopes on related sarbecovirus RBDs. We isolated and structurally characterized antibodies targeting both conserved and SARS-2-specific RBM epitopes, including a class of antibodies with broad sarbecovirus neutralization activity. The RBM-directed immune-focused response is potently neutralizing with breadth across SARS-2 variants and other coronaviruses without compromising SARS-2 neutralization or protection. Our data show how rationally designed immunogens can redirect immune responses to conserved coronavirus epitopes in the context of preexisting immunity.

### Results

Epitope Grafting of the SARS-CoV-2 Receptor Binding Motif onto Heterologous Coronavirus Scaffolds

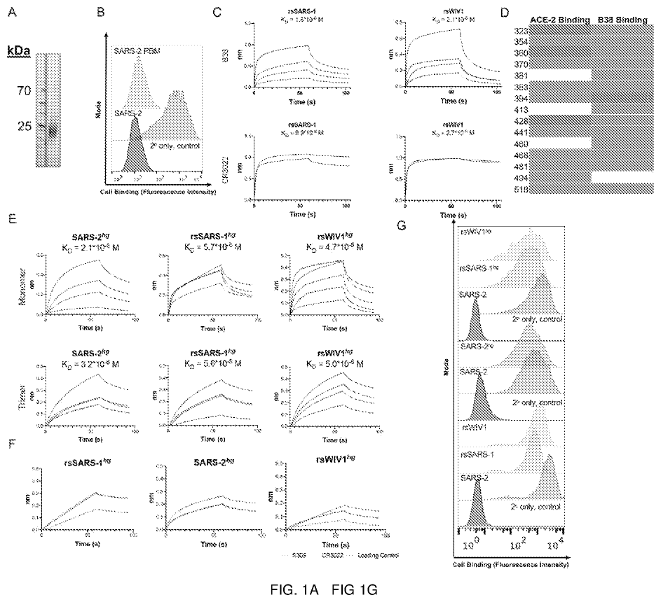

The RBM of SARS-2 and related sarbecoviruses, SARS-1 and WIV1-CoV (WIV1), is a contiguous sequence spanning residues 437-508 (SARS-2 numbering) of the spike protein. To elicit RBM-specific responses only, we first evaluated whether the RBM itself could be recombinantly expressed in absence of the rest of the RBD (FIG. 1A). While the SARS-2 RBM could indeed be expressed, it failed to engage the conformationally-specific RBM-directed antibody B38 or bind to cell-surface expressed ACE2 (FIG. 1B). These results indicate that the RBM is conformationally flexible, and that the RBD serves as a structural “scaffold” to stabilize the RBM in its binding-compatible conformation.

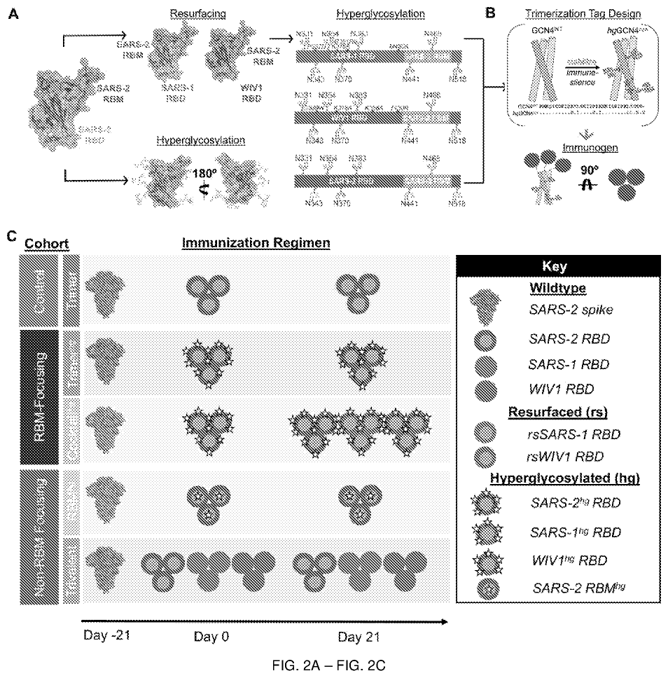

To circumvent the hurdle of de novo scaffold design to present the RBM, we tested whether heterologous sarbecovirus RBDs from SARS-1 and WIV1 and the more distantly related merbecovirus MERS-CoV (MERS) could serve as scaffolds (FIG. 2A). The SARS-1, WIV1, and MERS RBDs share a pairwise amino acid identity with SARS-2 of 73.0%, 75.4%, and 19.5%, respectively. Despite both using ACE2 as a receptor, the RBM is less conserved for SARS-1 and WIV1, with only 49.3% and 52.1% identity, respectively; as MERS uses DPP4 as a receptor, its RBM shares no notable identity (Raj et al., Nature 495, 251-254 (2013)). Although we were unable to “resurface” MERS RBD with the SARS-2 RBM, the related SARS-1 and WIV1 RBDs successfully accepted the RBM transfer. These resurfaced (rs) constructs, rsSARS-1 and rsWIV1, retained binding to the SARS-2 RBM-specific B38 antibody and efficiently engaged ACE2 (FIG. 1C) (Wu et al., Science 368, 1274-1278, (2020b)). These data indicate that there are sequence and structural constraints within the RBD required for successful RBM grafting; such an approach may be facilitated by using CoV RBDs that use the same receptor for viral entry.

### Engineered Glycans for Epitope Focusing

We next used these resurfaced RBDs as templates for further modification using glycan engineering. This approach aimed to mask conserved, cross-reactive epitopes shared between the SARS-1, SARS-2, and WIV1 RBDs to further enhance potential immune focusing to the RBM. There are two evolutionarily conserved predicted N-linked glycans (PNGs) at positions 331 and 343; SARS-1 and WIV1 have an additional conserved PNG at position 370 (SARS-2 numbering). To increase overall surface glycan density, we introduced novel PNGs onto wild-type SARS-2 as well as rsSARS-1 and rsWIV1 RBDs. Based on structural modeling and further biochemical validation, we identified 5 additional sites on rsSARS-1 and rsWIV1 and 6 on SARS-2. Including the native PNGs, all constructs had a total of 8 glycans (FIG. 2A, FIG. 1D-G)-we denoted these hyperglycosylated (hg) constructs as SARS-2^{hg}, rsSARS-1^{hg}, and rsWIV1^{hg}. We expressed these constructs in mammalian cells to ensure complex, heterogeneous glycosylation to maximize the glycan “shielding” effect. We subsequently characterized these constructs using the RBM-directed antibody B38, as well as ACE2 binding, to ensure that the engineered PNGs did not adversely affect the RBM conformation. Overall, the hyperglycosylated constructs were largely comparable in affinity for B38, with only ˜2-fold decrease, and still effectively engaged ACE2 (FIG. 1G). These results confirm a conformationally and functionally intact RBM.

Next, we assessed whether the engineered PNGs abrogated binding to sarbecovirus cross-reactive antibodies S309 and CR3022, which engage epitopes outside the RBM (Pinto et al., Nature 583, 290-295, (2020); Yuan et al., Science 368, 630-633, (2020)). The CR3022 epitope between SARS-1 and WIV1 differs at only a single residue whereas SARS-2 differs at 5 residues across both CR3022 and S309 epitopes (Wu et al., PLOS Pathog 16, e1009089, (2020)). Importantly, these epitope regions comprise a considerable portion of the non-RBM SARS-2 RBD, and thus any RBM focusing would require masking of these regions (Barnes et al., Nature 588, 682-687, (2020); Pinto et al., Nature 583, 290-295, (2020); Yuan et al., Science 368, 630-633, (2020)). While SARS-2^{hg} effectively abrogated S309 and CR3022 binding, the engineered PNGs at the antibody: antigen interface on rsSARS-1^{hg} and rsWIV1^{hg} did not completely abrogate S309 and CR3022 binding. We therefore incorporated unique mutations on rsSARS-1^{hg} and rsWIV1^{hg} so that any potentially elicited antibodies would be less likely to cross-react between these two constructs. To that end, we found K378A and the engineered glycan at residue 383 (SARS-2 numbering) abrogated CR3022 binding in both rsSARS-1^{hg} and rsWIV1^{hg} (FIG. 1F). For S309, mutations P337D in rsSARS-1^{hg} and G339W in rsWIV1^{hg} in addition to glycans at residues 441 and 354 (SARS-2 numbering) were sufficient to disrupt binding (FIG. 1F). We made two additional mutations, G381R, M430K on rsSARS-1^{hg} and K386A, T430R on rsWIV1^{hg}, to further increase the antigenic distance between these scaffolds (FIG. 2A).

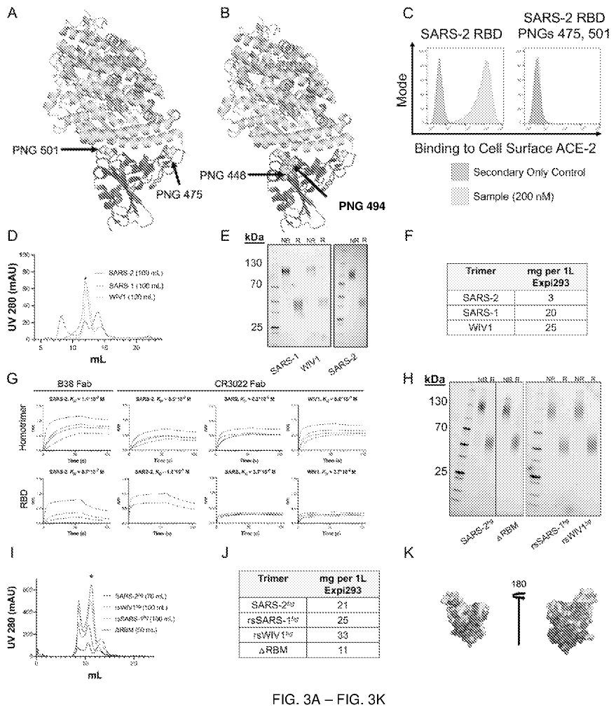

Lastly, we asked whether hyperglycosylation could similarly focus the humoral immune response to non-RBM epitopes. We therefore engineered a four novel glycans at positions 448, 475, 494, and 501 on the RBM of the wild-type SARS-2 RBD (RBM^{hg}) (FIG. 3A-B). The engineered PNGs effectively abrogate RBM-directed B38 antibody binding and engagement of ACE2 (FIG. 3C). Because the engineered PNGs restrict binding of RBM directed antibodies, this construct can also be used to assess RBM-focusing when compared to wild-type SARS-2 RBD.

### Design of a Non-Immunogenic Trimerization Tag for Enhanced Avidity

To increase avidity of our engineered immunogens while minimizing any off-target tag-specific responses, we designed a cysteine-stabilized and hyperglycosylated variant of a GCN4 trimerization tag (hgGCN4^{cys}) (FIG. 2B and Table 5) (Sliepen et al., J Biol Chem 290, 7436-7442, (2015)). While the two cysteines are within one subunit, they form an intermolecular disulfide with an adjacent subunit allowing the RBDs to remain trimerized while the tag is “immune silent”. We recombinantly expressed the engineered immunogens and wild-type RBD trimers in mammalian cells; the oligomeric state was confirmed using SDS-PAGE analysis under non-reducing conditions (FIG. 3D-F). Antigenicity was assayed using Fab fragments of conformationally-specific antibodies CR3022 and/or B38 using biolayer interferometry; the RBD trimers had comparable monovalent affinities in comparison to RBD monomers (FIG. 3G). We also used the hgGCN4^{cys} tag for the engineered hyperglycosylated and resurfaced immunogens (FIG. 3H-J).

### Cohorts and Immunization Regimens

We tested the immunogenicity and antigenicity of our designs (e.g., see Table 5) and assessed their RBM and non-RBM immune-focusing properties in wild-type mice (FIG. 2C). Cohorts are color-coded in FIG. 2C, and the same color-coding is used throughout all subsequent figures. All cohorts were primed with SARS-2 spike to reflect pre-existing SARS-2 immunity. All protein immunizations were adjuvanted with Sigma adjuvant. The control cohort was boosted with wild-type (i.e., unmodified) SARS-2 RBD trimer (“Trimer cohort”, grey). We divided our immune-focusing cohorts into RBM and non-RBM centered. For RBM immune-focusing, one cohort was boosted with SARS-2^{hg} trimers (“Trimer^{hg} cohort”, light blue), and a second cohort was boosted with SARS-2^{hg} trimers followed by a cocktail of rsSARS-1^{hg} and rsWIV1^{hg} (“Cocktail^{hg} cohort”, light green). For non-RBM immune-focusing, one cohort was boosted with RBM^{hg} trimers (“RBM^{hg}”, yellow), and a second was boosted with a cocktail of wild-type SARS-1, SARS-2, and WIV1 RBD trimers (“Trivalent cohort”, orange).

The Trimer^{hg} and RBM^{hg} cohorts described above use hyperglycosylation as an immune focusing strategy, whereas the Cocktail^{hg} cohort combines hyperglycosylation and resurfacing to enhance immune focusing to the RBM by reducing the prevalence of any non-RBM epitopes. The Trivalent cohort preferentially displays the conserved RBM across the wild-type SARS-2, SARS-1, and WIV1 RBDs; the majority of this conserved surface area falls outside the RBM (FIG. 3K).

### Immune Focusing of Serum Responses in the Context of SARS-CoV-2 Spike Imprinting

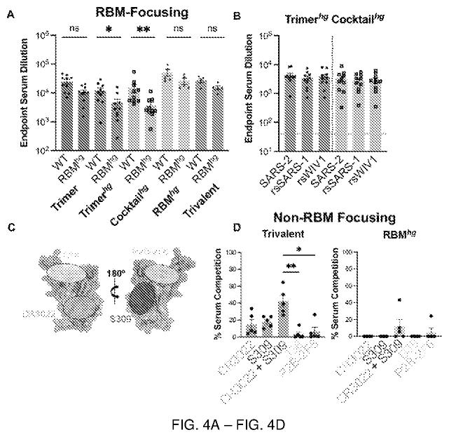

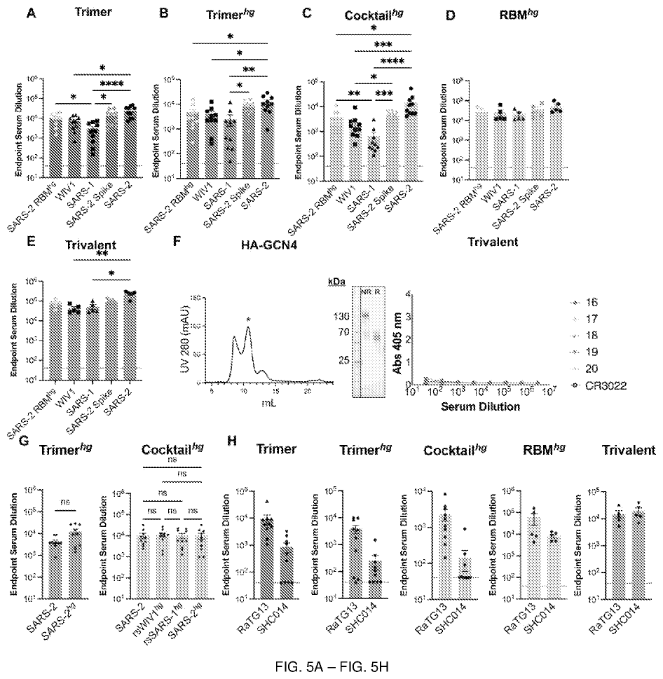

Across all cohorts, we observed a robust serum response to wild-type SARS-2 RBD (FIG. 4A, 5A-E) with minimal tag-directed responses (FIG. 5F). To specifically assess RBM-directed responses, we compared serum ELISA titers to wild-type SARS-2 RBD and our SARS-2 RBM^{hg} RBD construct; the latter has glycans that occlude the RBM. Notably, Trimer^{hg} and Cocktail^{hg} cohorts had a significant increase in serum antibody titers to wild-type SARS-2 RBD relative to SARS-2 RBM^{hg} RBD; this contrasted with the Trimer, RBM^{hg}, and Trivalent cohorts (FIG. 4A). Across the Trimer^{hg} and Cocktail^{hg} cohorts, the mean endpoint titer reduction to the SARS-2 RBM^{hg} RBD relative to wild-type SARS-2 RBD was ˜64%, which reflects a total or partial loss of affinity from antibodies due to steric interference by the RBM^{hg} engineered glycans. The Cocktail^{hg} cohort had a modest increase in RBM focusing relative to the Trimer^{hg} cohort. This may be due to increasing the overall antigenic distance (i.e., sequence difference) between the WIV1 and SARS-1 RBDs relative to SARS-2 while maintaining the identical SARS-2 RBM epitope. Additionally, we find that the Trimer^{hg} and Cocktail^{hg} cohorts had significantly lower titers to SARS-1 and WIV1 RBDs than to SARS-2 RBD (FIG. 5B, C). This difference was most pronounced in the Cocktail^{hg} cohort, indicating that the hyperglycosylation and engineered mutations within the RBD dampened responses to conserved, cross-reactive epitopes present outside the RBM. Furthermore, serum titers against the rsSARS-1 and rsWIV1 RBDs were comparable to SARS-2 RBD, indicating that there is minimal antibody response directed towards wild-type SARS-1 and WIV1 RBD epitopes in comparison to the SARS-2 RBM (FIG. 4B). We observed no significant glycan-dependent serum response in either the Trimer^{hg} or Cocktail^{hg} cohort (FIG. 5G). Collectively, these data confirm an enhanced SARS-2 RBM-focused serum response elicited by our engineered immunogens.

To determine whether the RBM^{hg} and Trivalent cohorts successfully directed the immune response to non-RBM epitopes on the RBD, we performed serum competition by incubating RBD-coated ELISA plates with B38, P2B-2F6, CR3022, and S309 IgGs, representing each of the four previously defined “classes” of SARS-CoV-2 RBD epitopes (FIG. 4C). Indeed, the previously characterized CR3022 and S309 antibodies have footprints that together cover much of the conserved non-RBM region of the RBD, with epitope buried surface area (BSA) of 917 Å2 and 795 Å2 respectively in comparison to BSA of 869 Å2 for ACE2 (Lan et al., Nature 581, 215-220, (2020); Pinto et al., Nature 583, 290-295, (2020); Yuan et al., Science 368, 630-633, (2020b)). We then assessed binding of mouse serum IgG (FIG. 4D). Only the Trivalent cohort showed a significant increase in serum competition when CR3022 and S309 IgGs are combined, indicating that only this regimen could effectively focus to conserved non-RBM epitopes on the SARS-2 RBD.

### Immunogen-Elicited Receptor Binding Motif-Focused Antibody Responses Potently Neutralize Sarbecoviruses

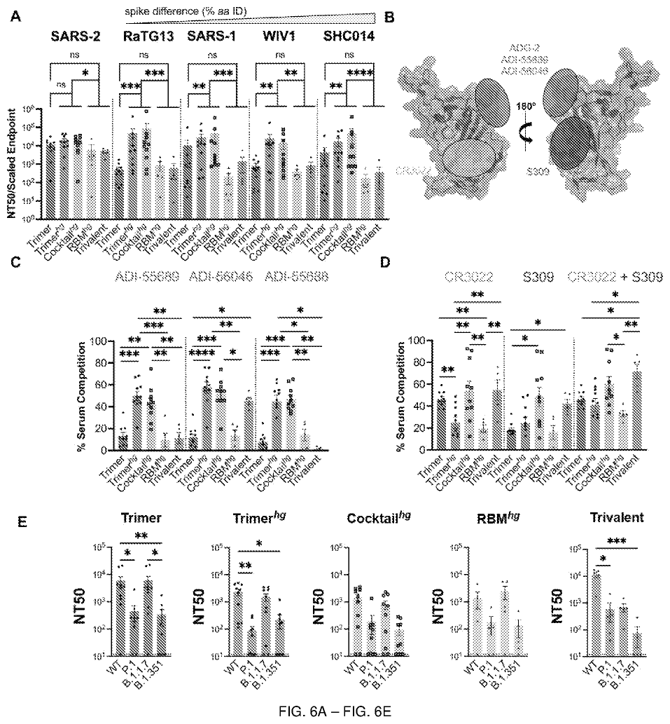

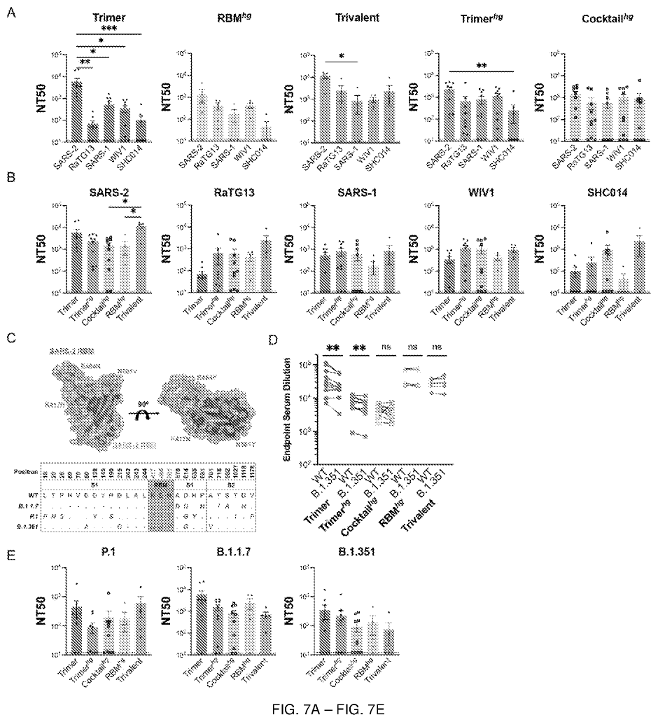

We next compared the neutralization potency (i. e., neutralization per unit of antigen-specific IgG) of all cohorts using SARS-1, SARS-2, WIV1, RaTG13-CoV (RaTG13), and SHC014-CoV (SHC014) pseudoviruses (Crawford et al., Viruses 12, (2020); Garcia-Beltran et al., Cell 184, 476-488 e411, (2021); Menachery et al., (2015); Shang et al., (2020)). WIV1, RaTG13, and SHC014 in this instance are broadly representative of possible future emerging sarbecoviruses with pandemic potential (Menachery et al., 2015; Menachery et al., Proc Natl Acad Sci USA 113, 3048-3053, 2016; Shang et al., Nature 581, 221-224, 2020). All cohorts elicited a potent SARS-2 neutralizing response. The RBM-focusing Trimer^{hg} and Cocktail^{hg} cohorts elicited a significantly more potent neutralizing response than the non-RBM focusing RBM^{hg} and Trivalent cohorts. Notably, the Trimer^{hg} and Cocktail^{hg} cohorts also neutralized SARS-1, WIV1, RaTG13, and SHC014 expressing pseudoviruses relative to the Trimer, RBM^{hg}, and Trivalent cohorts (FIG. 6A, 5A-E, H). This is noteworthy for the Trimer^{hg} cohort as it did not include any of these RBDs in the immunization regimen. Similarly, the Cocktail^{hg} cohort neutralized RaTG13 and SHC014, neither of which were present in the immunogen. In contrast, the Trimer cohort lost neutralization against RaTG13, SARS-1, WIV1, and SHC014, and the RBM^{hg} cohort trended towards a loss in neutralization as well (FIG. 7A, B). The Trivalent cohort also lost neutralization against SARS-1 and trended towards a loss against RaTG13, WIV1, and SHC014.

### Receptor Binding Motif-Focused Antibody Responses Target a Broadly Conserved Epitope

To further epitope map cross-reactive, RBM-focused serum responses, we performed ELISA-based antibody competition using cross-reactive antibodies CR3022, S309, ADI-55688, ADI-55689, and ADI-56046 with the WIV1 RBD (FIG. 6B-D). The latter three antibodies bind a conserved sarbecovirus RBM epitope also targeted by the antibody ADG-2, which is currently in clinical development and for which ADI-55688 is a precursor, as well as other antibodies with broad sarbecovirus neutralization (Martinez et al., bioRxiv, (2021); Rappazzo et al., Science 371, 823-829, (2021); Wec et al., Science 369, 731-736, (2020)). Competition ELISAs indicate that the cross-reactive WIV1-directed responses in the Trimer^{hg} and Cocktail^{hg} cohorts focus to the ADG-2-like epitope, as well as to the CR3022 and S309 epitopes in the Cocktail^{hg} cohort (FIG. 6C-D). Thus, SARS-2^{hg}, rsSARS-1^{hg}, and rsWIV1^{hg} RBDs not only can induce potent SARS-2 neutralizing antibodies, but also cross-reactive antibodies that bind to a conserved RBM epitope.

### Immune-Focused Responses Neutralize Variants of Concern

Many SARS-2 variants of concern include (see e.g., Table 4) mutations within the RBM including B.1.1.7 (Alpha), B.1.351 (Beta), and P.1 (Gamma) first detected in the United Kingdom, South Africa, and Brazil, respectively (FIG. 7C). We evaluated how enhanced focusing to the RBM affected binding to these variants. Serum from the Cocktail^{hg} cohort showed no significant loss of binding to the B.1.351 RBD compared to the wild-type SARS-2 RBD (FIG. 7D). In contrast, the Trimer and Trimer^{hg} cohorts had a significant loss of binding. The RBM^{hg} and Trivalent cohorts showed no significant loss of binding, consistent with a non-RBM focused serum antibody response.

Additionally, we tested all cohort sera for neutralization against B.1.1.7, B.1.351, and P.1 expressing pseudoviruses. While the Trimer and Trimer^{hg} cohorts still neutralized all pseudoviruses to some degree, there was reduced neutralization of P.1 and B.1.351, consistent with our ELISA data. The Trivalent cohort also showed reduced neutralization of P.1 and B.1.351 despite maintaining binding to the B.1.351 RBD in ELISA. In contrast, the Cocktail^{hg} and RBM^{hg} cohorts neutralized all variants (FIG. 6E). RBM^{hg} elicited responses likely were focused on neutralizing epitopes within the non-RBM RBD (e.g., CR3022/COVA1-16, S309 epitopes) and therefore were not sensitive to these RBM mutations (Liu et al., Immunity 53, 1272-1280 e1275, (2020a); Pinto et al., Nature 583, 290-295, (2020); Yuan et al., Science 368, 630-633, (2020)). However, the RBM-directed elicited response from the Cocktail^{hg} cohort maintained neutralization against all variants. This indicates that substantial immune-focusing to the RBM may allow for greater recognition (i.e., accommodation) of mutations compared to the RBM-directed antibody response elicited by natural infection or current vaccines (Yuan et al., Science. 73 (6556): 818-823, (2021); Zhou et al., Cell. 184 (9): 2348-2361, (2021)).

Additional Multimerization does not Improve SARS-CoV-2 Neutralization or Neutralization Breadth

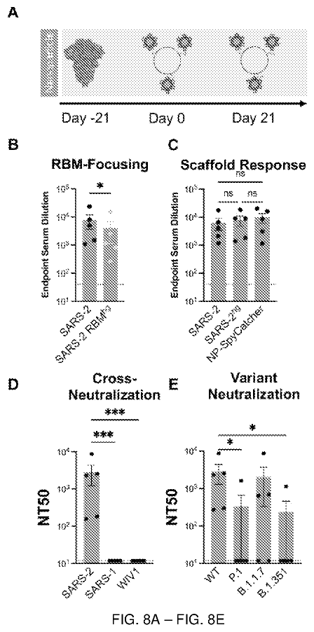

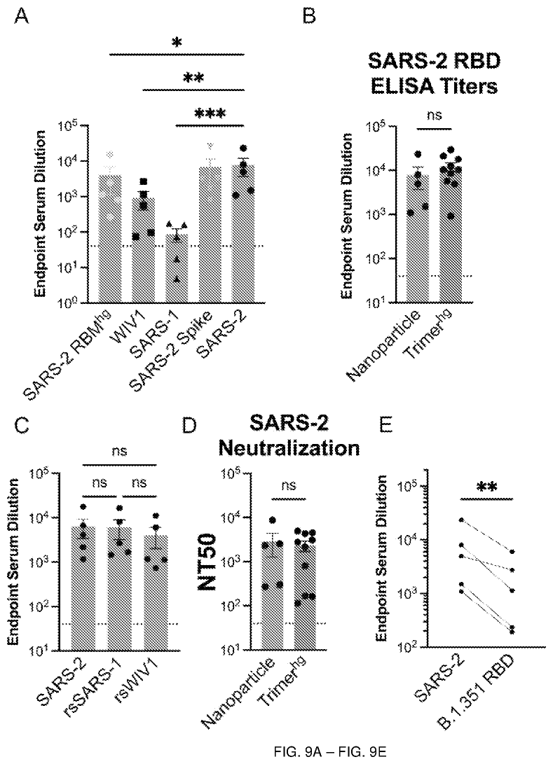

We evaluated whether further multimerization could improve the immunogenicity of our engineered immunogens. We created a SARS-2^{hg} RBD ferritin nanoparticle using SpyTag-SpyCatcher; the engineered RBD is the same used in our Trimer^{hg} cohort (FIG. 8A) (Zakeri et al., Proc Natl Acad Sci USA 109, E690-697, (2012)). We used the same immunization regimen as in the Trimer^{hg} cohort, allowing a direct comparison to the antigenicity and immunogenicity due to valency. Importantly, the nanoparticle immunogen did not elicit higher serum ELISA titers against the SARS-2 RBD (FIG. 9A, B), maintained RBM-focusing (FIG. 8B, C; FIG. 9C) and had comparable SARS-2 pseudovirus neutralization titers (FIG. 9D). However, the nanoparticle-boosted mice had markedly lower neutralization titers against SARS-1 and WIV1 pseudoviruses (FIG. 8D) and reduced neutralization of SARS-2 variants (FIG. 8E, FIG. 9E). These data indicate that at least for SARS-2^{hg} RBD, further multimerization using a ferritin nanoparticle confers minimal, if any, functional advantage.

Isolated Antibodies from Expanded IgG+ B Cell Lineages Include Antibodies with Broad Neutralization of Sarbecoviruses and Variants of Concern

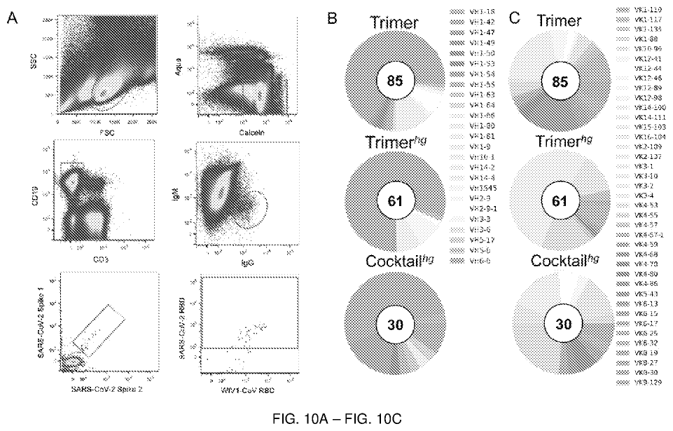

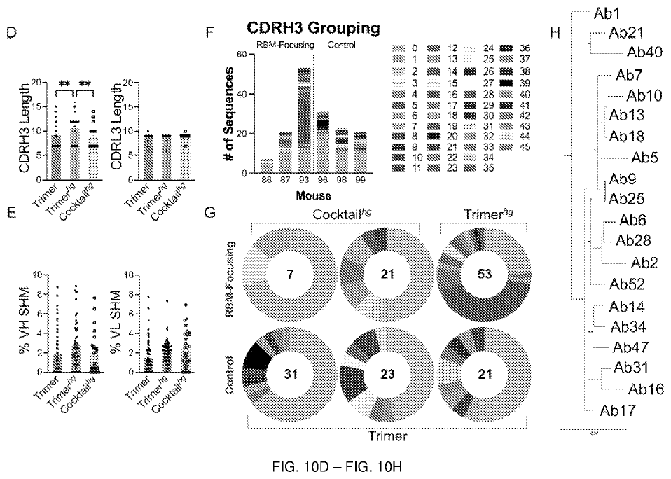

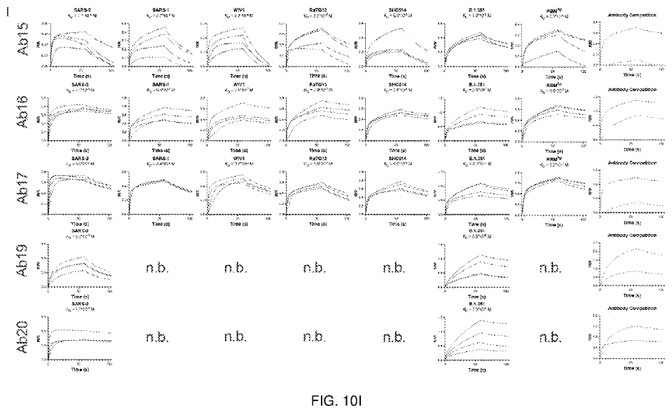

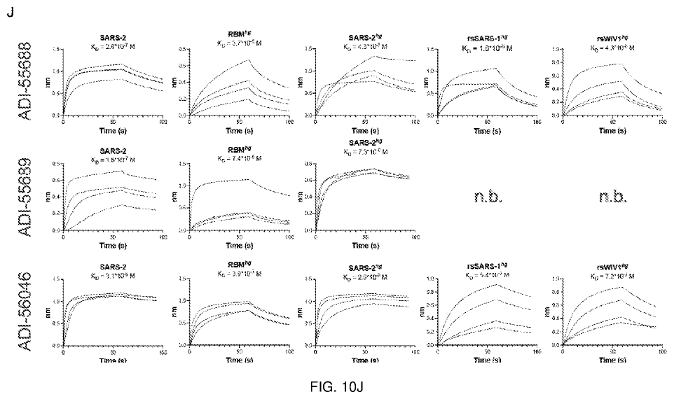

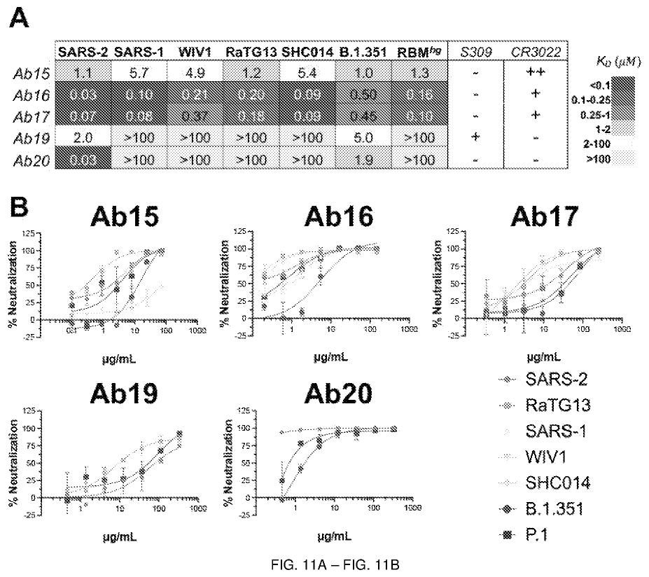

We next isolated a total of 85, 61, and 30 paired heavy and light chain sequences from SARS-2 RBD-specific IgG+ B cells from the Trimer, Trimer^{hg}, and Cocktail^{hg} cohorts, respectively (FIG. 10A). Overall, there was a predominance of IGHV1-42 gene usage across all cohorts, but light chain usage varied between the control Trimer cohort and the Trimer^{hg} and Cocktail^{hg} cohorts (FIG. 10B, C). CDRH3 length was significantly longer in the Trimer^{hg} cohort, with a median of 12 amino acids versus a median of 7 in both the Trimer and Cocktail^{hg} cohorts (FIG. 10D). Median somatic hypermutation was relatively similar between the cohorts (FIG. 10E). We chose 5 monoclonal antibodies from 4 different clonally related populations isolated from the Trimer^{hg} to express recombinantly for further characterization (FIG. 11A, FIG. 10F-I; Table 1). The CDRH3s of these clonally related populations were not shared with any antibodies isolated from the control Trimer cohort. Ab19 and Ab20 were SARS-2 specific, did not bind the RBM^{hg} construct, and did not compete with CR3022, indicating a largely RBM-directed epitope. Ab15 and the clonally related Ab16 and Ab17 (FIG. 10H) were exceptionally broad in their reactivity, engaging all coronavirus RBDs tested as well as the SARS-2 variant B.1.351 (FIG. 11A). These antibodies still bound the RBM^{hg} construct and either completely (Ab15) or partially (Ab16, Ab17) competed with CR3022; affinities to the B.1.351 and RBM^{hg} construct were between ˜2-20 fold lower than the affinity to the SARS-2 RBD. These data indicate a conserved epitope that partially overlaps both the CR3022 epitope and the RBM (FIG. 10J). In addition to their broad cross-reactivity, Abs 16 and 17 neutralized SARS-2, RaTG13, SARS-1, WIV1, and SHC014 (FIG. 11B). Furthermore, all antibodies neutralized P.1, and all except Ab19 neutralized B.1.351. This indicates that RBM-focusing can elicit antibodies capable of broadly neutralizing both related SARS-2 variants of concern and diverse sarbecoviruses.

Table 1 shows VH and VL sequences for antibodies selected for recombinant expression and characterization as shown in FIG. 11E.

TABLE 1

| Antibody | VH Sequence | VL Sequence |
|---|---|---|
| Ab15 | GAGGTCCAGCTGCAGCAGTCTGGACCTGAGC | GACATCCTGATGACCCAGTCTCCATCCTCCATG |
| | TGGTGAAGCCTGGGGCTTCAGTGAAGATATC | TCTGTATCTCTGGGAGACACAGTCAGCATCACT |
| | CTGCAAGGCTTCTGGTTACTCATTCACTGGC | TGCCATGCAAGTCAGGGCATTAGCAGTAATATA |
| | TACTACATGAACTGGGTGAAGCAAAGTCCTG | GGGTGGTTGCAGCAGAAACCAGGGAAATCATTT |
| | AAAAGAGCCTTGAGTGGATTGGAGAGATTAA | AAGGGCCTGATCTATCATGGAACCAACTTGGAA |
| | TCCTAACTTTGGTGGTACTACCTACAACCAG | GATGGAGTTCCATCAAGGTTCAGTGGCAGTGGA |
| | AAGTTCAAGGCCAAGGCCACATTGACTGTAG | TCTGGAGCAGCTTATTCTCTCACCATCAGCAGC |
| | ACAAATCCTCCAGCACAGCCTACATGCAGCT | CTGGAATCTGAAGATTTTGCAGACTATTACTGT |
| | CAAGAGCCTGACATCTGAGGACTCTGCAGTC | GTACAGTATACTCATTTTCCGTACACGTTCGGA |
| | TATTACTGTGCAAGATACTATGGTAACCTCT | GGGGGGACCAAGCTGGAAATAAAA (SEQ ID |
| | ATGCTATGGACTACTGGGGTCAAGGAACCTC | NO: 19) |
| | AGTCACCGTCTCC (SEQ ID NO: 18) | |
| Ab16 | GAGGTCCAGCTGCAGCAGTCTGGACCTGAGC | GACATCCTGATGACCCAATCTCCATCCTCCATG |
| | TGGTGAAGCCTGGGGCTTCAGTGAAGATATC | TCTGTATCTCTGGGAGACACAGTCAGCATCACT |
| | CTGCAAGGCTTCTGGTTACTCATTTAATAAC | TGCCATGCAAGTCAGGGCATTGGCAGTAATATA |
| | TACTACATGAACTGGGTGAAGCAGAGTCCTG | GGGTGGTTGCAGCAGAAACCAGGGAAATCATTT |
| | AAAAGAGCCTTGAGTGGATTGGAGAGATTAA | AAGGGCCTGATCTATCTTGGAACCAACTTGGAA |
| | TCCTAACTCTGGTTATACTTCCTACAACCAG | GATGGAGTTCCATCAAGGTTCAGTGGCAGTGGA |
| | AAGTTCAGGGCCAAGGCCACATTGACTGTAG | TCTGGAGCAGATTATTCTCTCACCATCAGCAGC |
| | ACAAATCCTCCACCACAGCCTACATGCAGCT | CTGGAATCTGAAGATTTTGCAGACTATTACTGT |
| | CAAGAGCCTGACATCTGAGGACTCTGCGGTC | GTACAGTATGTTCAGTTTCCGTACACGTTCGGA |
| | TATTACTGTGCAAGATACTTTGGTAACCTCT | GGGGGGACCAAGCTGGAAATAAAA (SEQ ID |
| | TTGCTATGGACTTCTGGGGTCAAGGAACCTC | NO: 21) |
| | AGTCACCGTCTCC (SEQ ID NO: 20) | |
| Ab17 | GAGGTCCAGCTGCAGCAGTCTGGACCTGAGC | GACATCCTGATGACCCAATCTCCATCCTCCATG |
| | TGGTGAAGCCTGGGGCTTCAGTGAAGATATC | TCTGTATCTCTGGGAGACACAGTCAGCATCACA |
| | CTGCAAGGCTTCTGGTTACTCATTCACTGAC | TGCCATGCAAGTCAGGGCATAAGTAGTAATATA |
| | TACTACATGAACTGGGTGAAGCAAAGTCCTG | GGGTGGTTGCAGCAGAAACCAGGGAAATCATTT |
| | AAAAGAGCCTTGAGTGGATTGGAGAGATTAA | AAGGGCCTGATCTATCATGGAACCAACTTGGAA |
| | TCCTAACACTGGTGGTACTACCTACAACCAG | GATGGAGTTCCATCAAGGTTCAGTGGCAGTGGA |
| | AAGTTCAAGGCCAAGGCCACATTGACTGTAG | TCTGGAGCAGATTATTCTCTCACCATCAGCAGC |
| | ACAAATCCTCCAGCACAGCCTACATGCAGCT | CTGGAATCTGAAGATTTTGCAGACTATTACTGT |
| | CAAGAGCCTGACATCTGAGGACTCTGCAGTC | GTACAGTATGTTCAGTTTCCGTACACGCTCGGA |
| | TATTACTGTGCAAGATACTATGGTAACCTCT | GGGGGGACCAAGCTGGAAATAAAA (SEQ ID |
| | ATGCTATGGACTACTGGGGTCAAGGAACCTC | NO: 23) |
| | AGTCACCGTCTCCTCA (SEQ ID NO: 22) | |
| Ab19 | CAGGTTCAGCTGCAGCAGTCTGGAGCTGAGC | GACATTGTGATGACCCAGTCTCACAAATTCATG |
| | TGGCGAGGCCTGGGGCTTCAGTGAAGCTGTC | TCCACATCAATAGGAGACAGGGTCAGCATCACC |
| | CTGCAAGGCTTCTGGCTACCCCTTCACAAGC | TGCAAGGCCAGTCACGATGTGAGTACTGCTGTA |
| | TATGGTATAAACTGGGTGAAGCAGAGAACTG | GCCTGGTATCAACAAAAACCAGGGCAATCTCCT |
| | GACAGGGCCTTGAGTGGATTGGAGAGATTTA | AAGTTACTGATTTACTGGGCATCCACCCGGCAC |
| | TCCTAGAATTGGAAATACTTACTATAATGAG | ACTGGAGTCCCTGATCGCTTCACAGGCAGTGGA |
| | AAGTTCAAGGGCAAGGCCACACTGACTGCAG | TCTGGGACAGATTATACTCTCACCATTAGAAGT |
| | ACAAATCCTCCAGCACAGCGTACATGGAGTT | GTGCAGGCAGAAGACCTGGCACTTTATTACTGT |
| | CCGCAGCCTGACATCTGAGGACTCTGCGGTC | CAGCAACATTATAGCACTCCGTACACGTTCGGA |
| | TATTTCTGTGCAAGATCGTGGAATAGTAACT | GGGGGGACCAAGCTGGAAATAAAA (SEQ ID |
| | ACGGGGAGTACTACTTTGACTACTGGGGCCA | NO: 25) |
| | AGGCACCACTCTCACAGTCTCC (SEQ ID | |
| | NO: 24) | |
| Ab20 | GAGGTCCAGCTGCAGCAGTCTGGACCTGAGC | GACATTGTGCTCACCCAATCTCCAGCTTCTTTG |
| | TGGTGAAGCCTGGGGCTTCAGTGAAGATATC | GCTGTGTCTCTAGGGCAGAGAGCCACCATCTCC |
| | CTGCAAGGCTTCTGGTTTCTCATTCACTGGC | TGCAGAGCCAGTGAAAGTGTTGAATATTATGGC |
| | TACTCCATGAACTGGATGAAACAAAGTCCTG | ACAGGTTTAGTGCAGTGGTTCCAACAGAAACCA |
| | AAAAGAGCCTTGAGTGGATTGGAGAAATTAA | GGACAGCCACCCAAACTCCTCATCTATGCTGCC |
| | TCCTACCACTGGTGGTACTACCTACAACCAG | TCCAACGTGGAATCTGGGGTCCCTGCCAGGTTT |
| | AAGTTCAAGGCCAAGGCCACATTGACTGTAG | AGTGGCAGTGGGTCTGGGACAGACTTCAGCCTC |
| | ACAAATCCTCCAGCACAGCCTACATACAACT | AACATCCATTCTGTGGAGGAGGATGATATTGCA |
| | CAAGAGCCTGACATCTGAGGACTCTGCAGTC | ATGTATTTCTGTCACCAAAGTAGGAAGCTTCCG |
| | TATTACTGTGCAAGGGGCCGGGCCGACTACT | TGGACGTTCGGTGGAGGCACCAAGCTGGAAAT |
| | GGGGCCAAGGCACCACTCTCACAGTCTCCTC | CAAA (SEQ ID NO: 27) |
| | A (SEQ ID NO: 26) | |

| Antibody | VH Sequence | VL Sequence |
|---|---|---|
| Ab15 | EVQLQQSGPELVKPGASVKISCKASGYSFTG | DILMTQSPSSMSVSLGDTVSITCHASQGISSNI |
| | YYMNWVKQSPEKSLEWIGEINPNFGGTTYNQ | GWLQQKPGKSFKGLIYHGTNLEDGVPSRFSGSG |
| | KFKAKATLTVDKSSSTAYMQLKSLTSEDSAV | SGAAYSLTISSLESEDFADYYCVQYTHFPYTFG |
| | YYCARYYGNLYAMDYWGQGTSVTVS (SEQ | GGTKLEIK (SEQ ID NO: 29) |
| | ID NO: 28) | |
| Ab16 | EVQLQQSGPELVKPGASVKISCKASGYSENN | DILMTQSPSSMSVSLGDTVSITCHASQGIGSNI |
| | YYMNWVKQSPEKSLEWIGEINPNSGYTSYNQ | GWLQQKPGKSFKGLIYLGTNLEDGVPSRFSGSG |
| | KFRAKATLTVDKSSTTAYMQLKSLTSEDSAV | SGADYSLTISSLESEDFADYYCVQYVQFPYTFG |
| | YYCARYFGNLFAMDFWGQGTSVTVS (SEQ | GGTKLEIK (SEQ ID NO: 31) |
| | ID NO: 30) | |
| Ab17 | EVQLQQSGPELVKPGASVKISCKASGYSFTD | DILMTQSPSSMSVSLGDTVSITCHASQGISSNI |
| | YYMNWVKQSPEKSLEWIGEINPNTGGTTYNQ | GWLQQKPGKSFKGLIYHGTNLEDGVPSRFSGSG |
| | KFKAKATLTVDKSSSTAYMQLKSLTSEDSAV | SGADYSLTISSLESEDFADYYCVQYVQFPYTLG |
| | YYCARYYGNLYAMDYWGQGTSVTVSS (SEQ | GGTKLEIK (SEQ ID NO: 33) |
| | ID NO: 32) | |
| Ab19 | QVQLQQSGAELARPGASVKLSCKASGYPFTS | DIVMTQSHKFMSTSIGDRVSITCKASHDVSTAV |
| | YGINWVKQRTGQGLEWIGEIYPRIGNTYYNE | AWYQQKPGQSPKLLIYWASTRHTGVPDRFTGSG |
| | KFKGKATLTADKSSSTAYMEFRSLTSEDSAV | SGTDYTLTIRSVQAEDLALYYCQQHYSTPYTFG |
| | YFCARSWNSNYGEYYFDYWGQGTTLTVS | GGTKLEIK (SEQ ID NO: 35) |
| | (SEQ ID NO: 34) | |
| Ab20 | EVQLQQSGPELVKPGASVKISCKASGFSFTG | DIVLTQSPASLAVSLGQRATISCRASESVEYYG |
| | YSMNWMKQSPEKSLEWIGEINPTTGGTTYNQ | TGLVQWFQQKPGQPPKLLIYAASNVESGVPARF |
| | KFKAKATLTVDKSSSTAYIQLKSLTSEDSAV | SGSGSGTDFSLNIHSVEEDDIAMYFCHQSRKLP |
| | YYCARGRADYWGQGTTLTVSS | WTFGGGTKLEIK (SEQ ID NO: 37) |
| | (SEQ ID NO: 36) | |

### Structural Characterization of Broadly Neutralizing Antibodies

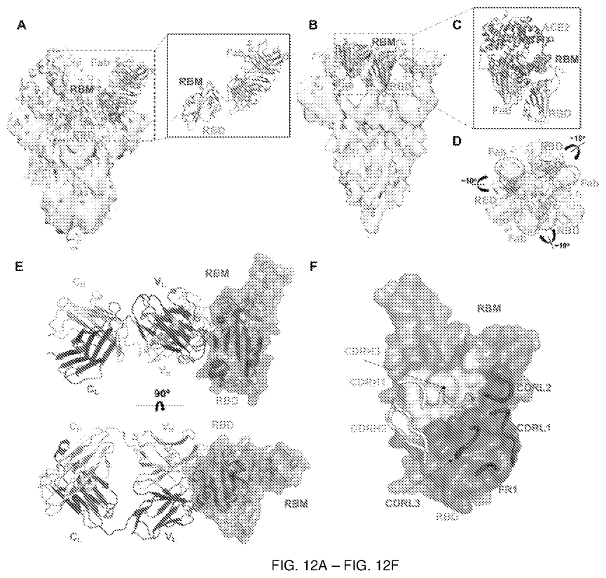

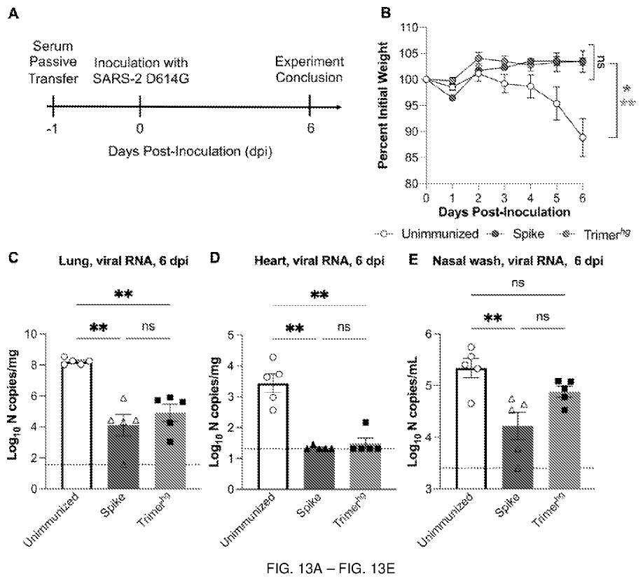

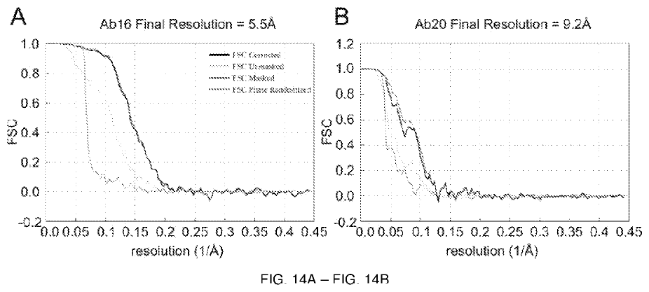

To define the epitope targeted by these Abs, we first obtained a low-resolution (9.2 Å) cryo-EM structure of Ab20 in complex with the SARS-2 spike (FIG. 12A). Ab20 appears to target an epitope on the upper loop of the SARS-2 RBM; its footprint likely will interfere with ACE2 binding and is consistent with the observation that Ab20 does not bind the RBM^{hg} with appreciable affinity. Moreover, Ab20 has a significant reduction in affinity for B.1.351 RBD relative to the wild-type SARS-2 RBD; mutation E484K within the RBM of B.1.351 appears to overlap with Ab20 footprint (FIG. 11E).

We next obtained a low resolution (5.5 Å) cryoEM structure of Ab16 in complex with SARS-2 spike (FIGS. 12B-D) and determined a high-resolution crystal structure of the clonally related Ab17 in complex with the SARS-2 RBD (FIGS. 12E-F). In the cryoEM structure with Ab16, the SARS-2 spike is in the “three RBD up” conformation with density for each RBD to be occupied by a Fab. Consistent with the reactivity from BLI, Ab16 appears to engage a conserved epitope that partially overlaps with the CR3022 epitope and includes part of the RBM, and Ab16 likely will sterically interfere with ACE2 binding (FIG. 12C). Furthermore, the complex appears to show an outward rotation of the bound RBD relative to the previously characterized “three RBD up” (PDB 7DX9) conformation (FIG. 12D). Indeed, this was previously hypothesized to contribute to SARS-1 neutralization by CR3022, and it may contribute to broad neutralization by Ab16 and Ab17 as well (Yuan et al., Science 368, 630-633, (2020b)). The higher-resolution co-crystal structure of Ab17 in complex with the SARS-2 RBD provides a more complete view of the antigen-combining site of these clonally related antibodies (FIG. 12E, Table 2). The crystal structure confirms a CR3022-overlapping epitope with additional interactions extending into the RBM. Furthermore, it overlaps with previously characterized conserved epitopes targeted by antibodies with broad sarbecovirus neutralization activity: antibody ADI-56046 from a human donor and antibodies K288.2 and K398.22 isolated from rhesus macaques (He et al., bioRxiv, (2021); Wec et al., Science 369, 731-736, (2020)). The overall footprint is large with a BSA of 1006 Å2 and includes interactions from CDR1-3 from both the heavy and light chains (FIG. 12F). Importantly, the Ab16 and Ab17 epitope is also left unmasked in the Trimer^{hg} and Cocktail^{hg} cohort boosting immunogens (FIG. 2A), allowing immune focusing to both conserved broadly neutralizing epitopes and the SARS-2 RBM.

Table 2 shows crystallographic data and refinement statistics. Statistics for the highest-resolution shell are shown in parentheses.

TABLE 2

| | Ab17 in complex with | |
|---|---|---|
| | SARS-2 RBD | |
| Wavelength (Å) | 0.9792 | |
| Resolution range (Å) | 74.66 − 3.5 | (3.625 − 3.5) |
| Space group | P 41 21 2 | |
| Unit cell (Å) | 207.931 207.931 86.662 | |
| (°) | 90 90 90 | |
| Total reflections | 513207 | (54030) |
| Unique reflections | 24538 | (2401) |
| Multiplicity | 20.9 | (22.5) |
| Completeness (%) | 99.98 | (100.00) |
| Mean I/sigma(I) | 13.89 | (3.37) |
| Wilson B-factor | 104.62 | |
| R_{merge} | 0.3361 | (1.898) |
| Rₘₑₐₛ | 0.3446 | (1.942) |
| Rₚᵢₘ | 0.07551 | (0.4066) |
| CC1/2 | 0.998 | (0.85) |
| CC* | 0.999 | (0.959) |
| Reflections used in refinement | 24537 | (2401) |
| Reflections used for R_{free} | 1206 | (118) |
| R_{work} | 0.296 | (0.339) |
| R_{free} | 0.345 | (0.405) |
| CC(work) | 0.799 | (0.738) |
| CC(free) | 0.777 | (0.583) |

| | |
|---|---|
| Protein residues | 1178 |
| RMS (bonds) (Å) | 0.003 |
| RMS (angles) (°) | 0.80 |
| Ramachandran favored (%) | 94.99 |
| Ramachandran allowed (%) | 4.83 |
| Ramachandran outliers (%) | 0.18 |
| Rotamer outliers (%) | 0.00 |
| Clashscore | 13.81 |
| Average B-factor | 95.58 |

### A SARS-CoV-2 Receptor Binding Motif-Focused Serum Response Protects Against SARS-CoV-2 Infection

Given that an RBM-focused immune response appears to be potently cross-neutralizing, we evaluated whether refocusing the immune response towards the SARS-2 RBM following imprinting with the SARS-2 spike was not inferior to maintaining an immune response directed towards the full SARS-2 spike. We compared protection against infection in K18-hACE2 transgenic mice with a SARS-2 virus containing the D614G mutation following passive transfer prior of sera collected at day 35 from the Trimer^{hg} cohort (FIG. 13A) (Winkler et al., Nat Immunol 21, 1327-1335, (2020)). For comparison, we used from mice that received SARS-2 spike protein (“Spike” cohort) three times, as well as unimmunized control mice (“Unimmunized” cohort). In comparison to the Unimmunized cohort, both the Trimer^{hg} and Spike immunization regimens conferred significant protection against weight loss (FIG. 13B). We also compared viral burden by analyzing viral RNA levels in the lung, heart, and nasal washes at 6 days post-infection. Within the lung and the heart, both the Spike and Trimer^{hg} sera conferred significant protection relative to the Unimmunized cohort (FIG. 13C, D). In nasal washes, there was a significant reduction in viral burden in the mice that received the Spike sera relative to the Unimmunized cohort, but the difference among the mice that received the Trimer^{hg} sera only trended towards significance (FIG. 13E). Across all metrics of protection, there was not a significant difference between Spike sera and Trimer^{hg} sera (FIGS. 13B-E). These data indicate that the Trimer^{hg} sera is relatively equivalent to the Spike sera in terms of protection conferred against severe SARS-2 infection and disease in mice and that refocusing the serum immune response towards the RBM may confer potent cross-neutralization sarbecoviruses without compromising protection against SARS-2.

The above-described results indicate that following an initial SARS-2 exposure (e.g., vaccination, infection), subsequent boosting with immunogens engineered to focus to conserved epitopes induces broad sarbecovirus immunity. Based on the data presented here, it would not occur at the expense of neutralizing activity against SARS-2. Although both the RBM-focused sera and the non-RBM focused sera showed reduced neutralization potency against SARS-2 variants P.1 and B.1.351, this effect was ameliorated somewhat relative to sera from the control Trimer cohort. Antibodies isolated from the Trimer^{hg} cohort neutralized both related sarbecoviruses and SARS-2 variants, and sera from the Cocktail^{hg} cohort retained neutralization against all variants tested. Notably, while we designed our RBM^{hg} and Trivalent cohorts to focus responses to RBD epitopes outside the RBM that are shared between SARS-2 and its variants, they still showed a reduction in neutralization potency. This indicates that mutations in P.1 and B.1.351 spikes outside the RBD also contribute to neutralization resistance, as both cohorts showed no significant loss of binding to the B.1.351 RBD.

Our study also shows how to use structure-guided hyperglycosylation and resurfacing to modulate the immune response. We showed how the former strategy used in the Trimer^{hg} and Cocktail^{hg} cohorts could direct responses to the SARS-2 RBM.

Collectively, our results demonstrate immunogen design approaches that can be leveraged to refocus antibody responses following SARS-2 spike imprinting. Importantly, these design strategies are not limited to coronaviruses and are adaptable to other viruses as a general approach to elicit protective responses to conserved epitopes. Refocusing to the SARS-2 RBM maintains protective SARS-2 neutralization while also eliciting antibody responses that recognize emerging variants and potently neutralize coronaviruses with pandemic potential.

The above results were obtained using the following materials and methods.

### Materials and Methods

### Immunogen and Coating Protein Expression and Purification

The SARS-CoV-2 (Genbank MN975262.1), SARS-CoV (Genbank ABD72970.1), WIV1-CoV (Genbank AGZ48828.1) RBDs were used as the basis for constructing these immunogens. To graft the SARS-2 RBM onto SARS-1 and WIV1 scaffolds to create the rsSARS-1 and rsWIV1 monomers, boundaries of SARS-2 residues 437-507 were used. All constructs were codon optimized by Integrated DNA Technologies and purchased as gblocks. Gblocks were then cloned into pVRC and sequence confirmed via Genewiz. Monomeric constructs for serum ELISA coating contained C-terminal HRV 3C-cleavable 8×His and SBP tags. Trimeric constructs also included C-terminal HRV 3C-cleavable 8×His tags, in addition to a hyperglycosylated GCN4 tag with two engineered C-terminal cystines modified from a previously published hyperglycosylated GCN4 tag (Sliepen et al., J Biol Chem 290, 7436-7442, 2015). Dr. Jason Mclellan at the University of Texas, Austin provided the spike plasmid, which contained a non-cleavable foldon trimerization domain in addition to C-terminal HRV 3C cleavable 6×His and 2× Strep II tags.

Expi 293F cells (ThermoFisher) were used to express proteins. Transfections were performed with Expifectamine reagents per the manufacturer's protocol. After 5-7 days, transfections were harvested and centrifuged for clarification. Cobalt-TALON resin (Takara) was used to perform immobilized metal affinity chromatography via the 8×His tag. Proteins were eluted using imidazole, concentrated, and passed over a Superdex 200 Increase 10/300 GL (GE Healthcare) size exclusion column. Size exclusion chromatography was performed in PBS (Corning). For immunogens, HRV 3C protease (ThermoScientific) cleavage of affinity tags was performed prior to immunization. Cobalt-TALON resin was used for a repurification to remove the His-tagged HRV 3C protease, cleaved tag, and remaining uncleaved protein.

### Fab and IgG Expression and Purification

The variable heavy and light chain genes for each antibody were codon optimized by Integrated DNA Technologies, purchased as gblocks, and cloned into pVRC constructs which already contained the appropriate constant domains as previously described (Schmidt et al., Cell Rep 13, 2842-2850, 2015a; Schmidt et al., Cell 161, 1026-1034, 2015b). The Fab heavy chain vector contained a HRV 3C-cleavable 8×His tag, and the IgG heavy chain vector contained HRV 3C-cleavable 8×His and SBP tags. The same transfection and purification protocol as used for the immunogens and coating proteins was used for the Fabs and IgGs.

### Biolayer Interferometry

Biolayer interferometry (BLI) experiments were performed using a BLItz instrument (Fortebio) with FAB2G biosensors or Ni-NTA biosensors (Fortebio). All proteins were diluted in PBS. Fabs were immobilized to the biosensors, and coronavirus proteins were used as the analytes. To determine binding affinities, single-hit measurements were performed starting at 10 UM to calculate an approximate K_{D} in order to evaluate which concentrations should be used for subsequent titrations. Measurements at a minimum of three additional concentrations were performed. Vendor-supplied software was used to generate a final K_{D} estimate via a global fit model with a 1:1 binding isotherm.

### Immunizations

All immunizations were performed using female C57BL/6 mice (Jackson Laboratory) aged 6-10 weeks. Mice received 20 μg of protein adjuvanted with 50% w/v Sigma adjuvant in 100 μL of inoculum via the intraperitoneal route. Following an initial prime (day-21), boosts occurred at days 0 and 21. Serum samples were collected for characterization on day 35 from all cohorts. All experiments were conducted with institutional IACUC approval (MGH protocol 2014N000252).

### Serum ELISAs

Serum ELISAs were executed using 96-well, clear, flat-bottom, high bind microplates (Corning). These plates were coated with 100 μL of protein, which were adjusted to a concentration of 5 μg/ml (in PBS). Plates were incubated overnight at 4° C. After incubation, plates had their coating solution removed and were blocked using 1% BSA in PBS with 1% Tween. This was done for 60 minutes at room temperature. This blocking solution was removed, and sera was diluted 40-fold in PBS. A 5-fold serial dilution was then performed. CR3022 IgG, similarly serially diluted (5-fold) from a 5 μg/mL starting concentration, was used as a positive control. 40 μL of primary antibody solution was used per well. Following this, samples were incubated for 90 minutes at room temperature. Plates were washed three times using PBS-Tween. 150 μL of HRP-conjugated rabbit anti-mouse IgG antibody, sourced commercially from Abcam (at a 1:20,000 dilution in PBS), was used for the secondary incubation. Secondary incubation was performed for one hour, similarly at room temperature. Plates were subsequently washed three times using PBS-Tween. 1×ABTS development solution (ThermoFisher) was used according to the manufacturer's protocol. Development was abrogated after 30 minutes using a 1% SDS solution, and plates were read using a SectraMaxiD3 plate reader (Molecular Devices) for absorbance at 405 nm.

### Competition ELISAs

A similar protocol to the serum ELISAs was used for the competition ELISAs. For the primary incubation, 40 μL of the relevant IgG at 1 μM was used at room temperature for 60 minutes. Mouse sera were then spiked in such that the final concentration of sera fell within the linear range for the serum ELISA titration curve for the respective coating antigen, and an additional 60 minutes of room temperature incubation occurred. After removing the primary solution, plates were washed three times with PBS-Tween. Secondary incubation consisted of HRP-conjugated goat anti-mouse IgG, human/bovine/horse SP ads antibody (Southern Biotech) at a concentration of 1:4000. The remaining ELISA procedure (secondary incubation, washing, developing) occurred as described for the serum ELISAs. Percent binding loss was calculated relative to a no IgG control. Negative percent binding loss values were set to zero for the purpose of visualizations.

### ACE2 Cell Binding Assay

ACE2 expressing 293T cells (Moore et al., J Virol 78, 10628-10635, 2004) (a kind gift from Nir Hacohen and Michael Farzan) were harvested. A wash was performed using PBS supplemented with 2% FBS. 200,000 cells were allocated to each labelling condition. Primary incubation occurred using 100 μL of 1 μM antigen in PBS on ice for 60 minutes. Two washes were performed with PBS supplemented with 2% FBS. Secondary incubation was performed using 50 μL of 1:200 streptavidin-PE (Invitrogen) on ice for 30 mins. Two washes were performed with PBS supplemented with 2% FBS, and then cells were resuspended in 100 μL of PBS supplemented with 2% FBS. A Stratedigm S1000Exi Flow Cytometer was used to perform flow cytometry. FlowJo (version 10) was used to analyze FCS files.

### Pseudovirus Neutralization Assay

Serum neutralization against SARS-CoV-2, SARS-CoV, WIV1-CoV, RaTG13, and SHC014 was assayed using pseudotyped lentiviral particles expressing spike proteins described previously (Garcia-Beltran et al., Cell 184, 476-488 e411, (2021a)). Transient transfection of 293T cells was used to generate lentiviral particles. Viral supernatant titers were measured using flow cytometry of 293T-ACE2 cells (Moore et al., 2004) and utilizing the HIV-1 p24CA antigen capture assay (Leidos Biomedical Research, Inc.). 384-well plates (Grenier) were used to perform assays on a Tecan Fluent Automated Workstation. For mouse sera, samples underwent primary dilutions of 1:3 or 1:9 followed by serial 3-fold dilutions. 20 μL each of sera and pseudovirus (125 infectious units) were loaded into each well. Plates were then incubated for 1 hour at room temperature. Following incubation, 10,000 293T-ACE2 cells (Moore et al., J Virol 78, 10628-10635, (2004)) in 20 μL of media containing 15 μg/mL polybrene was introduced to each well. The plates were then further incubated at 37° C. for 60-72 hours.

Cells were lysed using assay buffers described previously (Siebring-van Olst et al., J Biomol Screen 18, 453-461, (2013)). Luciferase expression was quantified using a Spectramax L luminometer (Molecular Devices). Neutralization percentage for each concentration of serum was calculated by deducting background luminescence from cells-only sample wells and subsequently dividing by the luminescence of wells containing both virus and cells. Nonlinear regressions were fitted to the data using GraphPad Prism (version 9), allowing IC50 values to be calculated via the interpolated 50% inhibitory concentration. IC50 values were calculated with a neutralization values greater than or equal to 80% at maximum serum concentration for each sample. NT50 values were then calculated using the reciprocal of IC50 values. Serum neutralization potency values were calculated by dividing the NT50 against a particular pseudovirus by the endpoint titer against the respective RBD. For samples with NT50 values below the limit of detection, the lowest limit of detection across all neutralization assays was used as the NT50 value to calculate neutralization potency. This prevents a higher limit of detection from skewing neutralization potency results. Endpoint titers were normalized relative to a CR3022 IgG control, which was run in every serum ELISA. ELISA titers that were too low to calculate an endpoint titer were set to 40, which was the starting point for the serum dilutions.

In comparing NT50 values for the various cohorts across the wild-type and variant pseudoviruses, the lowest limit of detection across all neutralization assays performed for a given cohort was used for any NT50 values that fell below the limit of detection. This prevents a higher limit of detection in some assays from skewing the comparison results.

### Flow Cytometry

Single cell suspensions were generated from mouse spleens following isolation via straining through a 70 μm cell strainer. Treatment with ACK lysis buffer was performed to remove red blood cells, and cells were washed with PBS. Aqua Live/Dead amine-reactive dye (0.025 mg/mL) was first used to stain single cell suspensions. The following B and T cell staining panel of mouse-specific antibodies was then applied: CD3-BV786 (BioLegend), CD19-BV421 (BioLegend), IgM-BV605 (BioLegend), IgG-PerCP/Cy5.5 (BioLegend). Staining was performed using a previously described staining approach (Sangesland et al., 2019; Weaver et al., (2016)).

SBP-tagged coronavirus proteins were labelled using streptavidin-conjugated flurophores as previously described (Kaneko et al., Cell 183, 143-157 e113, (2020)). Briefly, a final conjugated probe concentration of 0.1 μg/mL was achieved following the addition of streptavidin conjugates to achieve a final molar ratio of probe to streptavidin valency of 1:1. This addition was performed in 5 increments with 20 minutes of incubation at 4° C. with rotation in between. The coronavirus protein panel consisted of the following flurorescent probes: SARS-CoV-2 RBD-APC/Cy7 (streptavidin-APC/Cy7 from BioLegend), WIV1 RBD-BV650 (streptavidin-BV650 from BioLegend), SARS-CoV-2 spike-StreptTactin PE (StrepTactin PE from IBA Lifesciences), and SARS-CoV-2 spike-StreptTactin APC (StrepTactin APC from IBA Lifesciences).

A BD FACSAria Fusion cytometer (BD Biosciences) was used to perform flow cytometry. FlowJo (version 10) was used to analyze the resultant FCS files. Sorted cells were IgG+ B cells that were double-positive for SARS-CoV-2 spike and positive for the SARS-CoV-2 RBD.

### B Cell Receptor Sequencing

Cells were sorted into 96-well plates containing 4 μL of lysis buffer, consisting of 0.5×PBS, 10 mM DTT, and 4 units of RNaseOUT (ThermoFisher). Following sorting, plates were spun down at 3000 g for 1 minute and stored at −80° C. Plates were later thawed and a reverse transcriptase reaction was performed using the SuperScript IV VILO MasterMix (ThermoFisher) in a total volume of 20 μL according to the manufacturer's recommendations. Two rounds of PCR were then performed using previously published primers (Rohatgi et al., J Immunol Methods 339, 205-219, 2008; Tiller et al., J Immunol Methods 350, 183-193, 2009). Variable heavy and light chains were then sequenced via Sanger sequencing (Genewiz).

IMGT High V-Quest was used to analyze variable heavy and light chain sequences, and Cloanalyst was used to identify clonal lineages and to infer common ancestors in order to generate phylogenetic trees (Kepler et al., Front Immunol 5, (2014)). Data were plotted using Python and FigTree.

### Cryo-EM Grid Preparation and Image Recording

Complexes of SARS-CoV-2 spike (6P) with Ab16 Fab or Ab20 Fab were formed by combining spike at 0.7 mg/mL with Fab at 0.6 mg/ml (three-fold excess of binding sites) in a buffer composed of 10 mM Tris pH 7.5 with 150 mM NaCl (Hsieh et al., Science 369, 1501-1505, (2020)). Spike. Fab complexes were incubated for 30 minutes on ice before application to thick C-flat 1.2-1.3 400 Cu mesh grids (Protochips). Grids were glow discharged (PELCO easiGlow) for 30 seconds at 15 mA and prepared with a Gatan Cryoplunge 3 by applying 3.8 μL of sample and blotting for 4.0 seconds in the chamber maintained at a humidity between 88% and 92%. Images for Spike complexes with Ab16 or Ab20 were recorded on a Talos Arctica microscope operated at 200 keV with a Gatan K3 direct electron detector. Automated image acquisition was performed with Serial EM (Mastronarde, J Struct Biol 152, 36-51, (2005)).

### Cryo-EM Image Analysis and 3D Reconstruction and Model Fitting

Image analysis for was carried out in RELION as previously (Tong et al., Cell 184, 4969-4980 e4915, (2021)). Briefly, particles were extracted from motion-corrected micrographs and subjected to 2D classification, initial 3D model generation, 3D classification, and 3D refinement. Ab16 was C3 symmetric. CTF refinement was performed to correct beam tilt, trefoil, anisotropic magnification, and per particle defocus in RELION (Scheres, J Struct Biol 180, 519-530, (2012)). Bayesian polishing was also performed in RELION leading to a 6.6 Å reconstruction following 3D refinement. The final 3D refined map was sharpened with a B-factor of −297.5 Å² resulting in a 5.5 Å resolution map as determined by the Fourier shell correlation (0.143 cutoff) (FIG. 14A). Heavy and light chains of PDB entries 6LHQ and 4HC1 were aligned and extracted to make an initial model for the Fab. Spike with 3 RBD in the “up” conformation (PDB 7DX9) and model of Ab16 Fab were docked into the cryoEM map using Chimera (FIGS. 12B-D).

Micrographs from Ab20 in complex with spike were processed as above for Ab16. Most particles exhibited C1 symmetry due to conformational heterogeneity of the RBD relative to the S2 core of spike. Accordingly, particles with C3 symmetry were isolated by 3D classification for further refinement. CTF refinement was performed to correct beam tilt, trefoil, anisotropic magnification, and per particle defocus in RELION (Scheres, J Struct Biol 180, 519-530, (2012)). Bayesian polishing was also performed in RELION leading to a 10 Å reconstruction following 3D refinement. The final 3D refined map was sharpened with a B-factor of −768 Å2 resulting in a 9.2 Å resolution map as determined by the Fourier shell correlation (0.143 cutoff) (FIG. 14B). Heavy and light chains of PDB entries 4L5F and 4HC1 were aligned and extracted to make an initial model for the Fab. Spike with 3 RBD in the “down” conformation (PDB 6VXX) and model of Ab20 Fab were docked into the cryoEM map using Chimera (FIG. 12A).

The final reconstructions for Ab16 (EMD-24894) and Ab20 (EMD-24895) in complex with SARS-CoV-2 spike were deposited in the Electron Microscopy Data Bank.

### Crystallization

Ab17 and the SARS-2 RBD were incubated in a 1:1.2 molar ratio for 2 hours at 4° C. The resulting 1:1 complex was purified from excess SARS-2 RBD by gel filtration chromatography using a Superdex 200 Increase 10/300 GL (GE Healthcare) size exclusion column in 10 mM Tris-HCl, 150 mM NaCl, pH 7.5. The Ab17:SARS-2 RBD complex was concentrated to ˜13 mg/mL. Crystals grew in a hanging drop over a reservoir of 0.1 M HEPES pH 7.0 and 30% v/v Jeffamine@ ED-2001 pH 7.0 (Index screen condition D3, Hampton Research). Crystals were harvested, cryoprotected with additional crystallization buffer supplemented with MPD, and flash cooled using liquid nitrogen.

### Structure Determination and Refinement

Diffraction data were recorded at beamline 24-ID-E at the Advanced Photon source. Data were processed using XDS via the RAPD pipeline (Collaborative Computational Project, Acta Crystallogr D Biol Crystallogr 50, 760-763, (1994); Evans, Acta Crystallogr D Biol Crystallogr 62, 72-82, (2006); Evans, Acta Crystallogr D Biol Crystallogr 67, 282-292, (2011); Evans and Murshudov, Acta Crystallogr D Biol Crystallogr 69, 1204-1214, (2013); Kabsch, Acta Crystallogr D Biol Crystallogr 66, 133-144, (2010a); Acta Crystallogr D Biol Crystallogr 66, 125-132, (2010b)). Molecular replacement was performed using PHASER (McCoy et al., 2007). Refinement was performed using PHENIX, and model modifications were performed using COOT (Emsley and Cowtan, Acta Crystallogr D Biol Crystallogr 60, 2126-2132, (2004); Terwilliger et al., Acta Crystallogr D Biol Crystallogr 64, 61-69, (2008)). For the search model, a VHVL comprised of PDB entries 4L5F and 4HCl, respectively, with the CDRH3, CDRH2, and CDRL3 removed along with the SARS-2 RBD from PDB 6MOJ were used. The CDRH3, CDRH2, and CDRL3 were rebuilt de novo along with mutations in the V_{H}V_{L} using COOT, and refinement was performed using PHENIX. Refinement statistics are shown in Table 2.

### Animal Protection Experiments

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anesthesia that was induced and maintained with etamine hydrochloride and xylazine, and all efforts were made to minimize animal suffering.

K18-hACE2 C57BL/6J mice (strain: 2B6.Cg-Tg (K18-ACE2) 2Prlmn/J) were obtained from Jackson Laboratory (034860). Mice were administered 200 μL of pooled immune sera via intraperitoneal injection. One day after transfer, mice were inoculated with 10³ FFU of WA1/2020 SARS-CoV-2 strain with a D614G mutation via the intranasal route (Chen et al., Nat Med 27, 717-726, (2021)). Mice were monitored for weight loss, and daily weights were recorded. On day 6 post-inoculation, final weights were obtained, animals were sacrificed, and tissues were harvested.

### Measurement of Viral Burden

Tissues from each mouse were weighed. Homogenization was performed with sterile zirconia beads using a MagNa Lyser instrument (Roche Life Sciences) in 1 mL of DMEM supplemented with 2% heat-inactivated fetal bovine serum. Clarification was performed via centrifugation at 10,000 rpm for 5 min. Samples were stored at −80° C. RNA extraction was performed using the MagMax mirVana Total RNA isolation kit (Thermo Scientific) in combination with a Kingfisher Flex extraction machine (Thermo Scientific). RT-qPCR was then used to determine viral RNA levels as previously described (Hassan et al., Cell 182, 744-753 e744, (2020)). Viral RNA levels were normalized to tissue weight.

### Statistical Analysis

Curve fitting and statistical analyses were performed with GraphPad Prism (version 9). Non-parametric statistics were used throughout where feasible. Tests, numbers of animals, and statistical comparison groups are indicated in each of the Figure Legends. To compare multiple populations, the Kruskal-Wallis non-parametric ANOVA was used with post hoc analysis using Dunn's test for multiple comparisons. The Mann-Whitney U test was used to compare two populations without consideration for paired samples. The ratio-paired t-test was used to compare two populations with consideration for paired samples and evidence of normality. Analysis of weight change was determined by one-way ANOVA with Dunnett's test of area under the curve. P values in ANOVA analyses were corrected for multiple comparisons. A p value <0.05 was considered significant.

### II. Strategies for Immune Focusing

As is described below, we leveraged rational immunogen design strategies to focus humoral responses to the receptor binding motif (RBM) on the SARS-CoV-2 spike. Using glycan engineering and epitope scaffolding, we find an improved targeting of the serum response to the RBM in context of SARS-CoV-2 spike imprinting. Furthermore, we observed a robust SARS-CoV-2-neutralizing serum response with increased potency against related sarbecoviruses, SARS-CoV, WIV1-CoV, RaTG13-CoV, and SHC014-CoV. Thus, RBM focusing is a promising strategy to elicit breadth across emerging sarbecoviruses and represents an adaptable design approach for targeting conserved epitopes on other viral glycoproteins.

We have employed two immunogen design strategies used to direct humoral responses include “masking” epitopes via engineering putative N-linked glycosylation sites (PNGs) and the design of protein scaffolds to present broadly protective epitopes to the SARS-2 spike protein. This provides an opportunity to potentially improve serum neutralization potency, efficacy against variants, and cross-reactivity of antibody responses. A potential target of these efforts is the angiotensin converting enzyme 2 (ACE2) receptor binding motif (RBM) of the receptor binding domain (RBD) (Piccoli et al., Cell 183, 1024-1042 (2020); Barnes et al., Nature 588, 682-687 (2020)). Indeed, several potently neutralizing RBM-directed antibodies that interfere with ACE2 binding are protective and some can also neutralize related sarbecoviruses (Wec et al., Science 369, 731-736 (2020); Rappazzo et al., Science 371, 823-829 (2021); Barnes et al., Nature 588, 682-687 (2020); Wu et al., Science 368, 1274-1278 (2020); Hansen et al., Science 369, 1010-1014 (2020)). Below, we show that hyperglycosylation of the RBD and a “resurfacing” approach that grafts the RBM from SARS-2 onto heterologous coronavirus RBDs focuses serum responses to the RBM. This immune-focused response is potently neutralizing with breadth across SARS-2 variants and other coronaviruses.

Our results are described as follows.

### Resurfacing and Hyperglycosylation Approaches for Immune Focusing

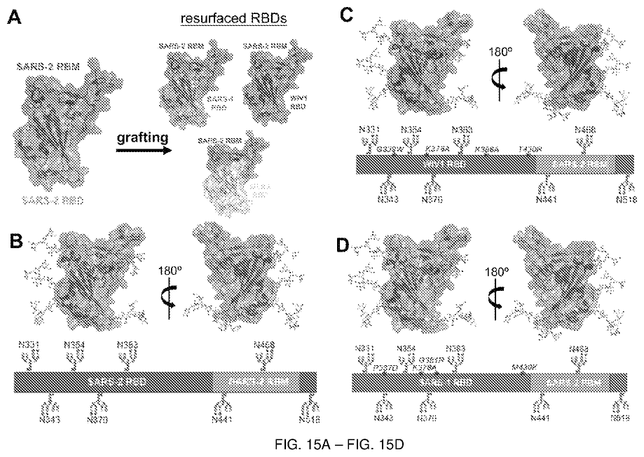

The RBM of SARS-2 and related sarbecoviruses, SARS-CoV (SARS-1) and WIV1-CoV (WIV1), is a contiguous sequence spanning residues 437-507 (SARS-2 numbering) of the spike protein. In an effort to elicit RBM-specific responses only, we first asked whether the RBM itself could be recombinantly expressed in absence of the rest of the RBD (FIG. 15A). While the SARS-2 RBM could indeed be overexpressed, it failed to both engage the conformationally-specific RBM-directed antibody B38 and bind to cell-surface expressed ACE2 (FIG. 1A-B). These results indicate that the RBM is conformationally flexible, and that the RBD serves as a structural “scaffold” to stabilize the RBM in its binding-compatible conformation. To circumvent the considerable hurdle of de novo scaffold design for RBM presentation, we asked whether heterologous sarbecovirus RBDs from SARS-1 and WIV1 and the more distantly related merbecovirus MERS-CoV (MERS) could serve as scaffolds (FIG. 15A). In context of immunizations, we hypothesized that these heterologous RBDs would present the SARS-2 RBM while removing any other SARS-2-specific epitopes. The SARS-1, WIV1 and MERS RBDs share a pairwise amino acid identity with SARS-2 of 73.0%, 75.4% and 19.5%, respectively. The RBM is less conserved despite have a shared ACE2 receptor for SARS-1 and WIV1 with only 49.3% and 52.1% identity, respectively; as MERS uses DPP4 as a receptor, its RBM shares no notable identity (Raj et al., Nature 495, 251-254 (2013)). While we were unable to “resurface” MERS RBD with the SARS-2 RBM, the related SARS-1 and WIV1 RBDs successfully accepted the RBM transfer. These resurfaced constructs, rsSARS-1 and rsWIV1 retained binding to the SARS-2 RBM-specific B38 antibody as well as effectively engaged ACE2 (FIG. 1C) (Wu et al., Science 368, 1274-1278 (2020)). These data indicate that there are sequence and structural constraints within the RBD required for successful RBM grafting; such an approach may be facilitated by using CoV RBDs that use the same receptor for viral entry.

We next used these resurfaced RBDs as templates for further modification using glycan engineering. This approach aimed to mask conserved, cross-reactive epitopes shared between the SARS-1, SARS-2, and WIV1 RBDs. There are two evolutionarily conserved PNGs at positions 331 and 343; SARS-1 and WIV1 have an additional conserved PNG at position 370 (SARS-2 numbering). To further increase overall surface glycan density, we introduced novel PNGs onto wildtype SARS-2 as well as rsSARS-1 and rsWIV1 RBDs. Based on structural modeling and biochemical validation, we identified 5 potential sites on rsSARS-1 and rsWIV1 as well as 6 on SARS-2. Including the native PNGs, all constructs had a total of 8 glycans (FIG. 1D, FIG. 3H-J, FIG. 15B-D)-we denote these hyperglycosylated (hg) constructs as SARS-2^{hg}, rsSARS-1^{hg}, and rsWIV1^{hg}. We expressed these constructs in mammalian cells to ensure complex glycosylation in order to maximize any glycan “shielding” effect. We subsequently characterized these constructs using the RBM-directed antibody B38, as well as ACE2 binding, to ensure that the engineered PNGs did not adversely affect the RBM conformation. Overall, the hyperglycosylated constructs were largely comparable in affinity for B38, with only ˜2-fold decrease, and still effectively engaged ACE2 (FIG. 1E-G). These results confirm a conformational and functionally intact RBM.

Next, we assessed whether the engineered PNGs abrogated binding to sarbecovirus cross-reactive antibodies S309 and CR3022-both antibodies were isolated from SARS-1 convalescent individuals (Pinto et al., Nature 583, 290-295 (2020); Yuan et al., Science 368, 630-633 (2020)). The CR3022 contact residues on SARS-1 and WIV1 differ only at a single residue while SARS-2 differs at 5 residues across both CR3022 and S309 epitopes (Wu et al., PLOS Pathog 16, e1009089 (2020)). Importantly, these epitopic regions were shown to be a significant portion of the SARS-2 RBD-directed response in murine immunizations and thus any RBM focusing would require masking of these regions (FIG. 1E-G) (Pinto et al., Nature 583, 290-295 (2020); Yuan et al., Science 368, 630-633 (2020); Hauser et al., bioRxiv, (2020)). While SARS-2^{hg} effectively abrogated S309 and CR3022 binding, the engineered PNGs at the antibody:antigen interface on rsSARS-1^{hg} and rsWIV1^{hg} did not completely abrogate S309 and CR3022 binding. We therefore incorporated unique mutations on rsSARS-1^{hg} and rsWIV1^{hg} so that any elicited antibodies would be less likely to cross-react between these two constructs. To that end, we found K378A and the engineered glycan at residue 383 (SARS-2 numbering) completely abrogated CR3022 binding in both rsSARS-1^{hg} and rsWIV1^{hg} (FIG. 1E-G). For S309, mutations P337D in rsSARS-1^{hg} and G339W in rsWIV1^{hg} in addition to glycans at residues 441 and 354 (SARS-2 numbering) were sufficient to disrupt binding (FIG. 1E-G). We made two additional mutations, G381R, M430K on rsSARS-1^{hg} and K386A, T430R on rsWIV1^{hg}, to further increase the antigenic distance between these scaffolds (FIG. 15C, D).

Serum Analysis from Cohorts

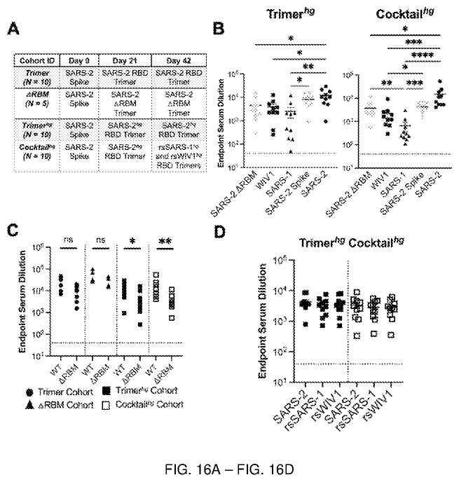

We then tested the immunogenicity and antigenicity of our optimized constructs and assessed their RBM immune-focusing properties, in the murine model. To increase avidity and to minimize any off-target tag-specific responses, we generated trimeric versions of each immunogen using a hyperglycosylated, cysteine-stabilized GCN4 tag (hgGCN4^{cys}). We first primed all cohorts with SARS-2 spike to reflect pre-existing SARS-2 immunity and to imprint an initial RBM response that may be recalled and selectively expanded by our immunogens. To test potential RBM immune-focusing, one cohort was sequentially immunized with SARS-2^{hg} trimers (“Trimer^{hg} cohort”) and a second cohort was immunized with SARS-2^{hg} trimers followed by a cocktail of rsSARS-1^{hg} and rsWIV1^{hg} (“Cocktail^{hg} cohort”) (FIG. 16A). In order to compare the efficacy of RBM-focusing, we included a “ΔRBM cohort”. This cohort was immunized with a modified SARS-2 RBD (ΔRBM) with four novel glycans engineered at positions 448, 475, 494, and 501 on the RBM. These PNGs effectively abrogate RBM-directed B38 antibody binding and engagement of ACE2 and should restrict elicited humoral responses to this epitope. Finally, as a control cohort, we included a SARS-2 spike prime followed with sequential immunizations with wildtype (i.e., unmodified) SARS-2 RBD trimer (“Trimer cohort”).

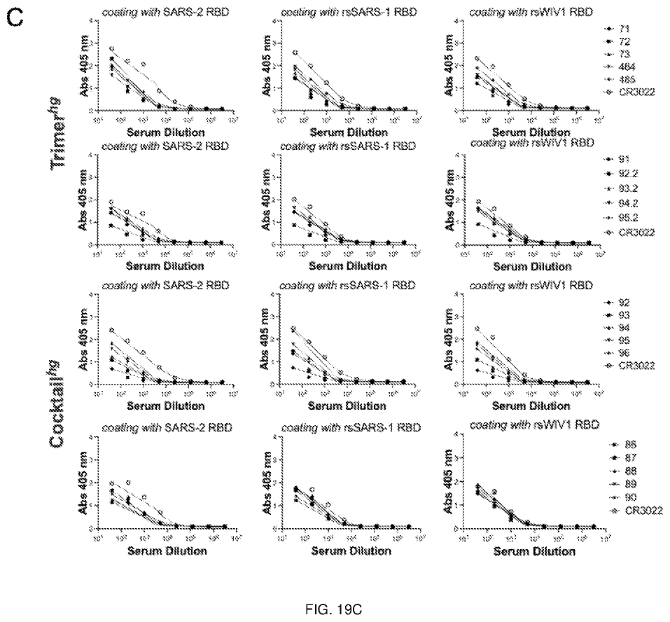

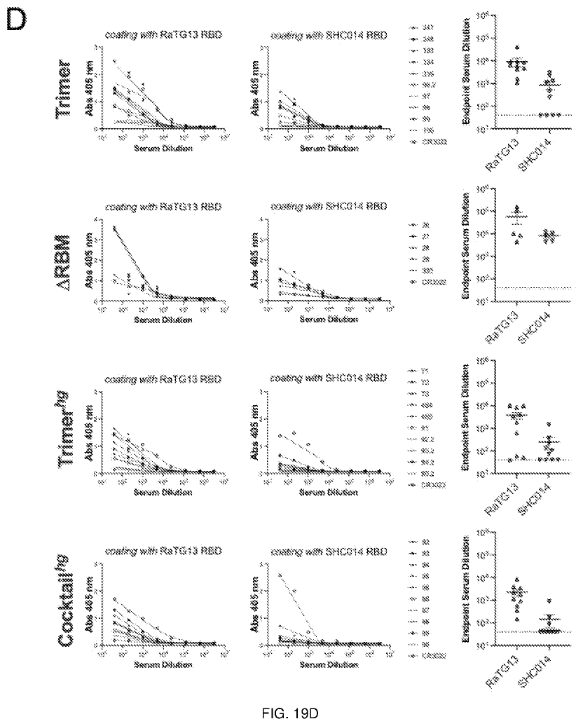

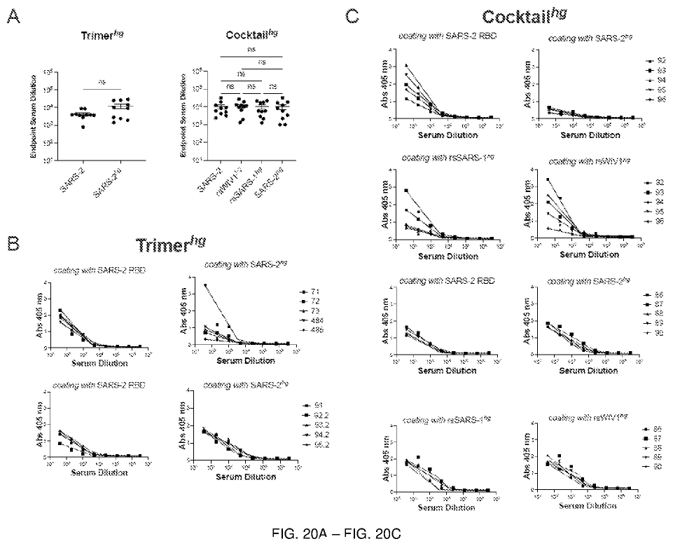

Overall, we found that all cohorts elicit robust serum responses to wildtype SARS-2 RBD (FIG. 16B-C, FIG. 1E-G). In order to specifically evaluate the RBM-directed responses, we compared serum ELISA titers to wildtype SARS-2 RBD and the SARS-2 ΔRBM RBD construct. We find that the Trimer^{hg} and Cocktail^{hg} cohorts had a significant increase in serum titers to wildtype SARS-2 RBD relative to SARS-2 ΔRBM RBD; this was in contrast to the ΔRBM and Trimer cohorts (FIG. 16B, C, FIG. 19A, B). Across the Trimer^{hg} and Cocktail^{hg} cohorts, the mean binding loss to the SARS-2 ΔRBM RBD relative to wildtype SARS-2 RBD was 64%, indicating that ˜64% of serum antibodies are RBM-directed by this metric (It is possible, however, that our engineered glycans ΔRBM construct does not fully restrict access by all RBM-directed responses with differing angles of approach). The Cocktail^{hg} cohort had a slight increase in RBM focusing relative to the Trimer^{hg} cohort. This may be due to increasing the overall antigenic distance (i.e., sequence difference) between the WIV1 and SARS-1 RBDs relative to SARS-2 while maintaining the identical SARS-2 RBM epitope. Additionally, we find that the Trimer^{hg} and Cocktail^{hg} cohorts had significantly lower titers to SARS-1 and WIV1 RBDs as compared to SARS-2 RBD (FIG. 16B). This difference was most pronounced in the Cocktail^{hg} cohort, indicating that the hyperglycosylation and engineered mutations within the RBD effectively dampened responses to these conserved, cross-reactive epitopes that are present outside the RBM. Furthermore, serum titers against the rsSARS-1 and rsWIV1 RBDs were comparable to SARS-2 RBD, indicating that there is minimal antibody response directed towards wildtype SARS-1 and WIV1 RBD epitopes in comparison to the SARS-2 RBM (FIG. 16D, FIG. 19C). We observed no significant glycan-dependent serum response in either cohort that used hyperglycosylation (FIG. 20). Collectively, these data confirm an enhanced SARS-2 RBM-focused serum response elicited by our engineered immunogens.

### Potency and Characterization of Neutralization

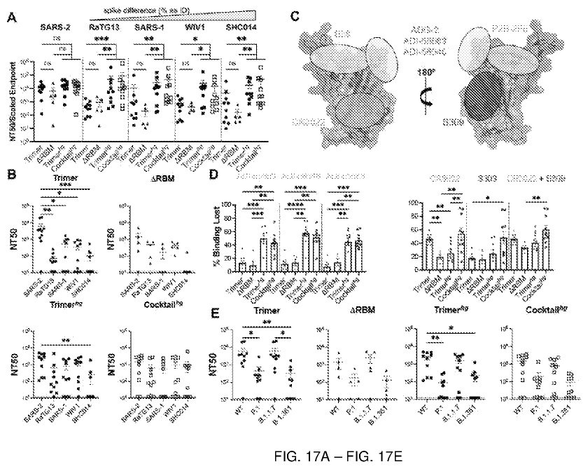

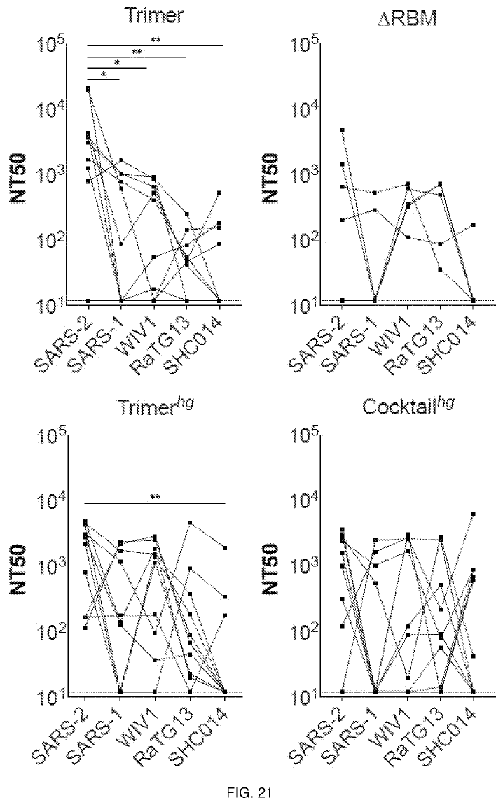

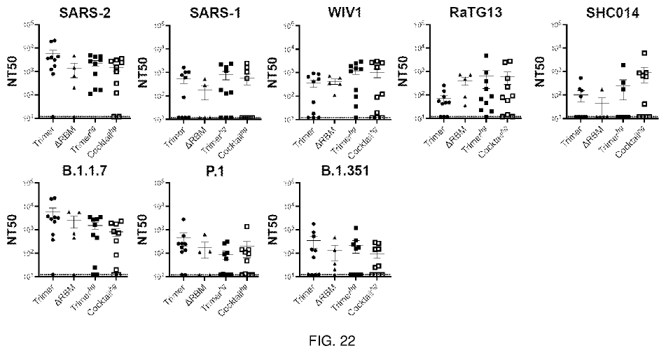

We next compared the neutralization potency of all cohorts using SARS-1, SARS-2, and WIV1 pseudoviruses, as well as RaTG13-CoV (RaTG13) and SHC014-CoV (SHC014) (Shang et al., Nature 581, 221-224 (2020); Garcia-Beltran et al., Cell 184, 476-488 e411 (2021); Crawford et al., Viruses 12, (2020); Menachery et al., Nat Med 21, 1508-1513 (2015)). While all cohorts elicited a potent SARS-2 neutralizing response, notably, the Trimer^{hg} and Cocktail^{hg} cohorts also exhibited potent SARS-1, WIV1, RaTG13, and SHC014 pseudovirus neutralization relative to the Trimer and ΔRBM cohorts (FIG. 17A, FIG. 21, FIG. 22, FIG. 19B, D). This is particularly noteworthy for the Trimer^{hg} cohort as it did not include SARS-1 or WIV1 RBDs in the immunization regimen. WIV1, RaTG13, and SHC014 in this instance are broadly representative of possible future emerging sarbecoviruses with pandemic potential (Shang et al., Nature 581, 221-224 (2020); Menachery et al., Nat Med 21, 1508-1513 (2015); Menachery et al., Proc Natl Acad Sci USA 113, 3048-3053 (2016)). The Trimer cohort lost significant neutralization against RaTG13, SARS-1, WIV1, and SHC014, and the ΔRBM trended towards a loss in neutralization as well, mirroring patterns seen following SARS-2 infection and immunization in humans, as well as SARS-2 spike-based immunization in mice (Garcia-Beltran et al., Cell, (2021); Garcia-Beltran et al., Cell 184, 476-488 e411 (2021); He et al., bioRxiv, (2021)). While the Trimer^{hg} cohort had a significant loss in neutralization against the most genetically divergent sarbecovirus, SHC014, it retained potency against RaTG13, SARS-1, and WIV1. Importantly, the Cocktail^{hg} had no significant loss in neutralization against either RaTG13 or SHC014, neither of which are vaccine-matched strains (FIG. 17B, FIG. 21, FIG. 22).

To further epitope map the RBM-focused responses, we performed ELISA-based antibody competition using cross-reactive antibodies CR3022, S309, ADI-55688, ADI-55689, and ADI-56046 and WIV1 RBD (FIG. 17C-D). The latter two antibodies bind a conserved sarbecovirus RBM epitope also targeted by the antibody ADG-2, which is currently in clinical development and for which ADI-55688 is a precursor, and other antibodies with broad sarbecovirus neutralization (Wec et al., Science 369, 731-736 (2020); Rappazzo et al., Science 371, 823-829 (2021); Martinez et al., bioRxiv, (2021)). Competition ELISAs indicate that the cross reactive WIV1-directed responses in the Trimer^{hg} and Cocktail^{hg} cohorts focus to the ADG-2-like epitope, as well as to the CR3022 and S309 epitopes in the Cocktail^{hg} cohort (FIGS. 17C-D). Thus, SARS-2^{hg}, rsSARS-1^{hg}, and rsWIV1^{hg} RBDs can induce not only potent SARS-2 neutralizing antibodies, but also cross-reactive antibodies that bind to a conserved RBM epitope (FIG. 10J). Notably, these results are in contrast to our previous work showing that a cocktail of sarbecovirus that included SARS-1 and WIV1 RBDs could predominantly focus the antibody response towards the conserved CR3022 and S309 epitopic regions.

### Binding of SARS-2 Variants

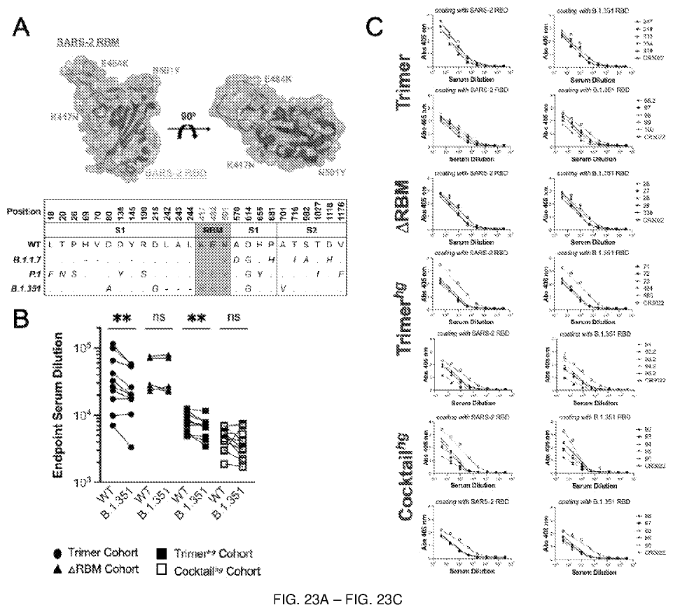

Many SARS-2 variants of concern include mutations within the RBM including B.1.1.7, B.1.351 and P.1 first detected in the United Kingdom, South Africa, and Brazil, respectively (FIG. 23A). We therefore asked what the consequence was of enhanced focusing to the RBM and whether the elicited responses elicited were sensitive to these mutations. Interestingly, serum from the Cocktail^{hg} cohort showed no significant loss of binding to the B.1.351 RBD compared to the wildtype SARS-2 RBD (FIG. 23B). This is in contrast to the control Trimer cohort and the Trimer^{hg} cohort, which showed a significant loss of binding and parallels the observation of reduced serum binding from human subjects immunized with current SARS-2 vaccines (Garcia-Beltran et al., Cell, (2021); Zhou et al., Cell, (2021); Wang et al., Nature 592, 616-622 (2021) 8, 38, 39). Second, we tested all sera for neutralization against SARS-2 variant pseudoviruses: B.1.1.7, B.1.351 and P.1. While the control Trimer cohort and the Trimer^{hg} cohort could still neutralize all pseudoviruses, there was a significant loss of neutralization to the P.1 and B.1.351 variants, consistent with our ELISA data. In contrast, we find no significant loss of neutralization against these variants in the Cocktail^{hg} and ΔRBM cohorts (FIG. 17E). For ΔRBM cohort, the elicited responses were likely focused to neutralizing epitopes within the non-RBM RBD (e.g., CR3022, S309) and therefore were not sensitive to these RBM mutations. However, the neutralizing response observed in the Cocktail^{hg} cohort potentially indicates that substantial immune-focusing to the RBM may allow for greater recognition (i.e., accommodation) of mutations compared to the RBM-directed antibody response elicited via infection or vaccination (Zhou et al., Cell, (2021); Yuan et al., Science, (2021)).

### SARS-2 RBD-Directed B Cell Characteristics

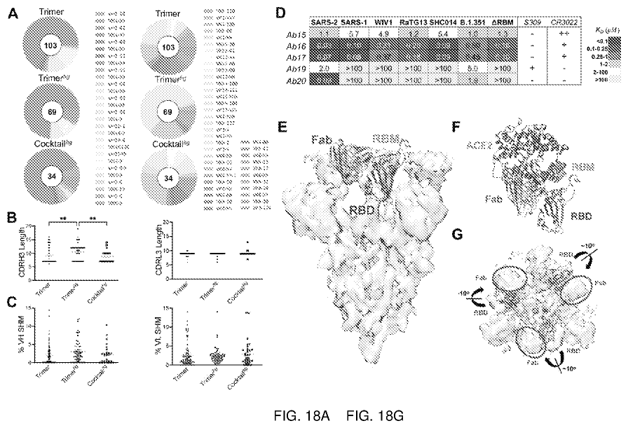

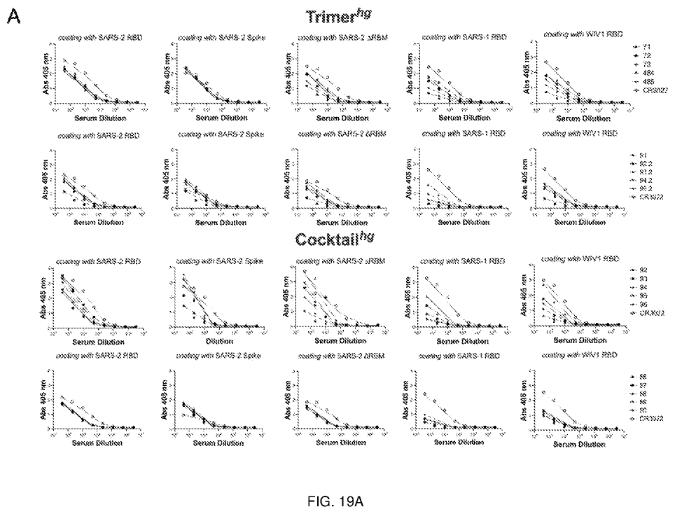

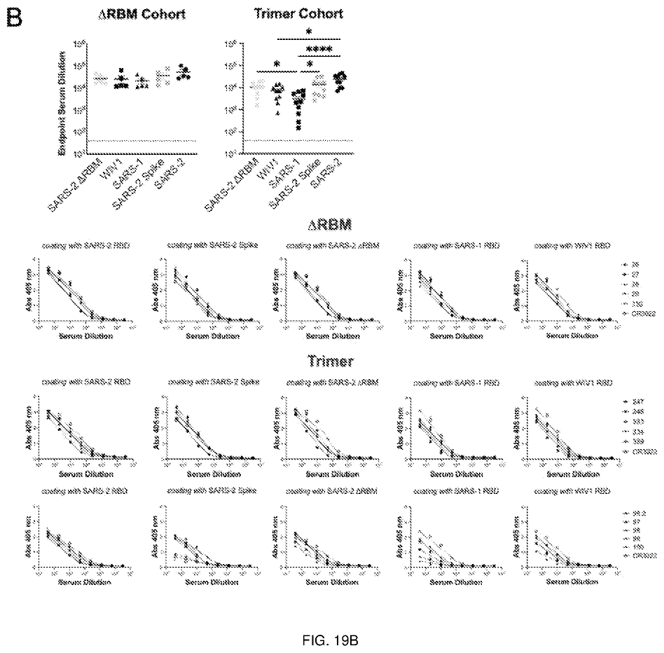

We next isolated SARS-2 RBD-specific IgG+ B cells from the Trimer, Trimer^{hg}, and Cocktail^{hg} cohorts and obtained paired heavy and light chain sequences (FIG. 10A). Overall, there was a predominance of IGHV1-42 gene usage across all cohorts, but light chain usage patterns varied more noticeably between the control Trimer cohort and the Trimer^{hg} and Cocktail^{hg} cohorts (FIG. 18A). CDRH3 length was significantly longer in the Trimer^{hg} cohort; mean somatic hypermutation trended towards being higher in the Trimer^{hg} and Cocktail^{hg} cohorts compared to the Trimer cohort (FIG. 18B-C). We recombinantly expressed representative antibodies from clonally related populations from the Trimer^{hg} and Cocktail^{hg} cohorts to test for breadth and to crudely epitope map (FIG. 18D, FIG. 10I; Table 1). Ab19 and Ab20 were SARS-2 specific, did not bind the ΔRBM construct and did not compete with CR3022, indicating an RBM-focused epitope. Importantly, Ab15, Ab16, and Ab17 were exceptionally broad in their reactivity, engaging all coronavirus RBDs tested as well as the SARS-2 variant B.1.351 (FIG. 18D). These antibodies still bound the ΔRBM construct and either completely (Ab15) or partially (Ab16, Ab17) competed with CR3022; affinities to the B.1.351 and ΔRBM construct were between ˜2-20 fold lower than the affinity to the SARS-2 RBD. These data indicate a conserved epitope that partially overlaps both the CR3022 epitope and the RBM (FIG. 10J).

To further define the epitope targeted by these Abs, we obtained a low-resolution cryo-EM structure of Ab16 in complex with the SARS-2 spike (FIG. 18E-G). The SARS-2 spike is in the “three RBD up” conformation with density for each RBD to be occupied by a Fab. Consistent with the reactivity in BLI, Ab16 appears to engage a conserved epitope that partially overlaps with the CR3022 epitope and encompasses part of the RBM (FIG. 18E). This latter observation will likely sterically interfere with ACE2 binding (FIG. 18F). Furthermore, the complex appears to show an outward rotation of the bound RBD relative to the previously characterized “three RBD up” (PDB 7DX9) conformation (FIG. 18G). Indeed, this was previously hypothesized to contribute to SARS-1 neutralization by CR3022 (28). The Ab16 binding footprint appears to overlap with previously characterized conserved epitopes targeted by antibodies with broad sarbecovirus neutralization activity: ADI-56046 and antibodies K288.2 and K398.22 isolated from rhesus macaques (Wec et al., Science 369, 731-736 (2020); He et al., bioRxiv, (2021)). This region is also left unmasked in the Trimer^{hg} and Cocktail^{hg} cohort boosting immunogens (FIG. 15), allowing immune focusing to both conserved broadly neutralizing epitopes and the SARS-2 RBM.

Collectively, our results demonstrate immunogen design approaches that can be leveraged to enhance RBD, and more specifically, RBM-focused humoral responses. It is a strategy that maintains protective SARS-2 neutralization while also eliciting humoral responses that recognize emerging variants and coronaviruses with pandemic potential. Importantly, these design strategies are not limited to coronaviruses and are adaptable to other viruses as a general approach to elicit protective responses to conserved epitopes.

The above results were obtained using the following materials and methods.

### Materials and Methods

### Immunogen and Coating Protein Expression and Purification

The SARS-CoV-2 (Genbank MN975262.1), SARS-CoV (Genbank ABD72970.1), WIV1-CoV (Genbank AGZ48828.1) RBDs were used as the basis for constructing these immunogens. To graft the SARS-2 RBM onto SARS-1 and WIV1 scaffolds to create the rsSARS-1 and rsWIV1 monomers, boundaries of SARS-2 residues 437-507 were used. All constructs were codon optimized by Integrated DNA Technologies and purchased as gblocks. Gblocks were then cloned into pVRC and sequence confirmed via Genewiz. Monomeric constructs for serum ELISA coating contained C-terminal HRV 3C-cleavable 8×His and SBP tags. Trimeric constructs also included C-terminal HRV 3C-cleavable 8×His tags, in addition to a previously published hyperglycosylated GCN4 tag with two engineered C-terminal cystines (Hauser et al., bioRxiv, (2020); Sliepen et al., J Biol Chem 290, 7436-7442 (2015)). Dr. Jason Mclellan at the University of Texas, Austin provided the spike plasmid, which contained a non-cleavable foldon trimerization domain in addition to C-terminal HRV 3C cleavable 6×His and 2× Strep II tags. The SARS-2 ΔRBM RBD construct was generated as previously described with four additional engineered putative N-linked glycosylation sites at positions 448, 475, 494, and 501.

Expi 293F cells (ThermoFisher) were used to express proteins. Transfections were performed with Expifectamine reagents per the manufacturer's protocol. After 5-7 days, transfections were harvested and centrifuged for clarification. Cobalt-TALON resin (Takara) was used to perform immobilized metal affinity chromatography via the 8×His tag. Proteins were eluted using imidazole, concentrated, and passed over a Superdex 200 Increase 10/300 GL (GE Healthcare) size exclusion column. Size exclusion chromatography was performed in PBS (Corning). For immunogens, HRV 3C protease (ThermoScientific) cleavage of affinity tags was performed prior to immunization. Cobalt-TALON resin was used for a repurification to remove the His-tagged HRV 3C protease, cleaved tag, and remaining uncleaved protein.

### Fab and IgG Expression and Purification

The variable heavy and light chain genes for each antibody were codon optimized by Integrated DNA Technologies, purchased as gblocks, and cloned into pVRC constructs which already contained the appropriate constant domains as previously described (Schmidt et al., Cell Rep 13, 2842-2850 (2015); Schmidt et al., Cell 161, 1026-1034 (2015)). The Fab heavy chain vector contained a HRV 3C-cleavable 8×His tag, and the IgG heavy chain vector contained HRV 3C-cleavable 8×His and SBP tags. The same transfection and purification protocol as used for the immunogens and coating proteins was used for the Fabs and IgGs.

### Biolayer Interferometry

Biolayer interferometry (BLI) experiments were performed using a BLItz instrument (Fortebio) with FAB2G biosensors or Ni-NTA biosensors (Fortebio). All proteins were diluted in PBS. Fabs were immobilized to the biosensors, and coronavirus proteins were used as the analytes. To determine binding affinities, single-hit measurements were performed starting at 10 μM to calculate an approximate K_{D} in order to evaluate which concentrations should be used for subsequent titrations. Measurements at a minimum of three additional concentrations were performed. Vendor-supplied software was used to generate a final K_{D} estimate via a global fit model with a 1:1 binding isotherm.

### Immunizations

All immunizations were performed using female C57BL/6 mice (Jackson Laboratory) aged 6-10 weeks. Mice received 20 μg of protein adjuvanted with 50% w/v Sigma adjuvant in 100 μL of inoculum via the intraperitoneal route. Following an initial prime (day 0), boosts occurred at days 21 and 42. Serum samples were collected for characterization on day 56 from all cohorts, in addition to day 35 for the Trimer^{hg} and Cocktail^{hg} cohorts. All experiments were conducted with institutional IACUC approval (MGH protocol 2014N000252).

### Serum ELISAs

Serum ELISAs were executed using 96-well, clear, flat-bottom, high bind microplates (Corning). These plates were coated with 100 μL of protein, which were adjusted to a concentration of 5 μg/ml (in PBS). Plates were incubated overnight at 4° C. After incubation, plates had their coating solution removed and were blocked using 1% BSA in PBS with 1% Tween. This was done for 60 minutes at room temperature. This blocking solution was removed, and sera was diluted 40-fold in PBS. A 5-fold serial dilution was then performed. CR3022 IgG, similarly serially diluted (5-fold) from a 5 μg/mL starting concentration, was used as a positive control. 40 μL of primary antibody solution was used per well. Following this, samples were incubated for 90 minutes at room temperature. Plates were washed three times using PBS-Tween. 150 μL of HRP-conjugated rabbit anti-mouse IgG antibody, sourced commercially from Abcam (at a 1:20,000 dilution in PBS), was used for the secondary incubation. Secondary incubation was performed for one hour, similarly at room temperature. Plates were subsequently washed three times using PBS-Tween. 1× ABTS development solution (ThermoFisher) was used according to the manufacturer's protocol. Development was abrogated after 30 minutes using a 1% SDS solution, and plates were read using a SectraMaxiD3 plate reader (Molecular Devices) for absorbance at 405 nm.

### Competition ELISAs

A similar protocol to the serum ELISAs was used for the competition ELISAs. For the primary incubation, 40 μL of the relevant IgG at 1 μM was used at room temperature for 60 minutes. Mouse sera were then spiked in such that the final concentration of sera fell within the linear range for the serum ELISA titration curve for the respective coating antigen, and an additional 60 minutes of room temperature incubation occurred. After removing the primary solution, plates were washed three times with PBS-Tween. Secondary incubation consisted of HRP-conjugated goat anti-mouse IgG, human/bovine/horse SP ads antibody (Southern Biotech) at a concentration of 1:4000. The remaining ELISA procedure (secondary incubation, washing, developing) occurred as described for the serum ELISAs. Percent binding loss was calculated relative to a no IgG control. Negative percent binding loss values were set to zero for the purpose of visualizations.

### ACE2 Cell Binding Assay

ACE2 expressing 293T cells (Moore et al., J Virol 78, 10628-10635 (2004)) (a kind gift from Nir Hacohen and Michael Farzan) were harvested. A wash was performed using PBS supplemented with 2% FBS. 200,000 cells were allocated to each labelling condition. Primary incubation occurred using 100 μL of 1 μM antigen in PBS on ice for 60 minutes. Two washes were performed with PBS supplemented with 2% FBS. Secondary incubation was performed using 50 L of 1:200 streptavidin-PE (Invitrogen) on ice for 30 mins. Two washes were performed with PBS supplemented with 2% FBS, and then cells were resuspended in 100 μL of PBS supplemented with 2% FBS. A Stratedigm S1000Exi Flow Cytometer was used to perform flow cytometry. FlowJo (version 10) was used to analyze FCS files.

### Pseudovirus Neutralization Assay

Serum neutralization against SARS-CoV-2, SARS-CoV, WIV1-COV, RaTG13, and SHC014 was assayed using pseudotyped lentiviral particles expressing spike proteins described previously (Garcia-Beltran et al., Cell 184, 476-488 e411 (2021)). Transient transfection of 293T cells was used to generate lentiviral particles. Viral supernatant titers were measured using flow cytometry of 293T-ACE2 cells (Moore et al., J Virol 78, 10628-10635 (2004)) and utilizing the HIV-1 p24CA antigen capture assay (Leidos Biomedical Research, Inc.). 384-well plates (Grenier) were used to perform assays on a Tecan Fluent Automated Workstation. For mouse sera, samples underwent primary dilutions of 1:3 or 1:9 followed by serial 3-fold dilutions. 20 μL each of sera and pseudovirus (125 infectious units) were loaded into each well. Plates were then incubated for 1 hour at room temperature. Following incubation, 10,000 293T-ACE2 cells (Moore et al., J Virol 78, 10628-10635 (2004)) in 20 μL of media containing 15 μg/mL polybrene was introduced to each well. The plates were then further incubated at 37° C. for 60-72 hours.

Cells were lysed using assay buffers described previously (Siebring-van Olst et al., J Biomol Screen 18, 453-461 (2013)). Luciferase expression was quantified using a Spectramax L luminometer (Molecular Devices). Neutralization percentage for each concentration of serum was calculated by deducting background luminescence from cells-only sample wells and subsequently dividing by the luminescence of wells containing both virus and cells. Nonlinear regressions were fitted to the data using GraphPad Prism (version 9), allowing IC50 values to be calculated via the interpolated 50% inhibitory concentration. IC50 values were calculated with neutralization values greater than or equal to 80% at maximum serum concentration for each sample. NT50 values were then calculated using the reciprocal of IC50 values. Serum neutralization potency values were calculated by dividing the NT50 against a particular pseudovirus by the endpoint titer against the respective RBD. For samples with NT50 values below the limit of detection, the lowest limit of detection across all neutralization assays was used as the NT50 value to calculate neutralization potency. This prevents a higher limit of detection from skewing neutralization potency results. Endpoint titers were normalized relative to a CR3022 IgG control, which was run in every serum ELISA. ELISA titers that were too low to calculate an endpoint titer were set to 40, which was the starting point for the serum dilutions.

In comparing NT50 values for the various cohorts across the wildtype and variant pseudoviruses, the lowest limit of detection across all neutralization assays performed for a given cohort was used for any NT50 values that fell below the limit of detection. This prevents a higher limit of detection in some assays from skewing the comparison results.

### Flow Cytometry

Single cell suspensions were generated from mouse spleens following isolation via straining through a 70 μm cell strainer. Treatment with ACK lysis buffer was performed to remove red blood cells, and cells were washed with PBS. Aqua Live/Dead amine-reactive dye (0.025 mg/mL) was first used to stain single cell suspensions. The following B and T cell staining panel of mouse-specific antibodies was then applied: CD3-BV786 (BioLegend), CD19-BV421 (BioLegend), IgM-BV605 (BioLegend), IgG-PerCP/Cy5.5 (BioLegend). Staining was performed using a previously described staining approach (Sangesland et al., Immunity 51, 735-749 e738 (2019); Weaver et al., Nat Protoc 11, 193-213 (2016)).

SBP-tagged coronavirus proteins were labelled using streptavidin-conjugated fluorophores as previously described (Kaneko et al., Cell 183, 143-157 e113 (2020)). Briefly, a final conjugated probe concentration of 0.1 μg/mL was achieved following the addition of streptavidin conjugates to achieve a final molar ratio of probe to streptavidin valency of 1:1. This addition was performed in 5 increments with 20 minutes of incubation at 4° C. with rotation in between. The coronavirus protein panel consisted of the following fluorescent probes: SARS-CoV-2 RBD-APC/Cy7 (streptavidin-APC/Cy7 from BioLegend), WIV1 RBD-BV650 (streptavidin-BV650 from BioLegend), SARS-CoV-2 spike-StreptTactin PE (StrepTactin PE from IBA Lifesciences), and SARS-CoV-2 spike-StreptTactin APC (StrepTactin APC from IBA Lifesciences).

A BD FACSAria Fusion cytometer (BD Biosciences) was used to perform flow cytometry. FlowJo (version 10) was used to analyze the resultant FCS files. Sorted cells were IgG+ B cells that were double-positive for SARS-CoV-2 spike and positive for the SARS-CoV-2 RBD.

### B Cell Receptor Sequencing

Cells were sorted into 96-well plates containing 4 μL of lysis buffer, consisting of 0.5×PBS, 10 mM DTT, and 4 units of RNaseOUT (ThermoFisher). Following sorting, plates were spun down at 3000 g for 1 minute and stored at −80° C. Plates were later thawed and a reverse transcriptase reaction was performed using the SuperScript IV VILO MasterMix (ThermoFisher) in a total volume of 20 μL according to the manufacturer's recommendations. Two rounds of PCR were then performed using previously published primers (Rohatgi et al., J Immunol Methods 339, 205-219 (2008); Tiller et al., J Immunol Methods 350, 183-193 (2009)). Variable heavy and light chains were then sequenced via Sanger sequencing (Genewiz).

IMGT High V-Quest was used to analyze variable heavy and light chain sequences, and IgBlast was used to identify clonal lineages. Data were plotted using Python.

### Cryo-EM Grid Preparation and Image Recording

Complexes of SARS-CoV-2 spike (6P) with Ab16 Fab were formed by combining spike at 0.7 mg/mL with Fab at 0.6 mg/mL (three-fold excess of binding sites) in a buffer composed of 10 mM Tris pH 7.5 with 150 mM NaCl. Spike. Fab complexes were incubated for 30 minutes on ice before application to thick C-flat 1.2-1.3 400 Cu mesh grids (Protochips). Grids were glow discharged (PELCO easiGlow) for 30 seconds at 15 mA and prepared with a Gatan Cryoplunge 3 by applying 3.8 μL of sample and blotting for 4.0 seconds in the chamber maintained at a humidity between 88% and 92%. Images for Spike complexes with Ab16 were recorded on a Talos Arctica microscope operated at 200 keV with a Gatan K3 direct electron detector. Automated image acquisition was performed with Serial EM (Mastronarde et al., J Struct Biol 152, 36-51 (2005)).

### Cryo-EM Image Analysis and 3D Reconstruction and Model Fitting

Image analysis for was carried out in RELION as previously. Briefly, particles were extracted from motion-corrected micrographs and subjected to 2D classification, initial 3D model generation, 3D classification, and 3D refinement. 2D class averages are shown in FIG. 24. Ab16 was C3 symmetric. CTF refinement was performed to correct beam tilt, trefoil, anisotropic magnification, and per particle defocus in RELION (Scheres et al., J Struct Biol 180, 519-530 (2012)). Bayesian polishing was also performed in RELION leading to a 6.6 Å reconstruction following 3D refinement. The final 3D refined map was sharpened with a B-factor of −297.5 Å2 resulting in a 5.5 Å resolution map as determined by the Fourier shell correlation (0.143 cutoff). Heavy and light chains of PDB entries 4L5F and 4HC1 were aligned and extracted to make an initial model for the Fab. Spike with 3 RBD in the “up” conformation (PDB 7DX9) and model of Ab16 Fab were docked into the cryoEM map using Chimera.

### Statistical Analysis

Curve fitting and statistical analyses were performed with GraphPad Prism (version 9). Non-parametric statistics were used throughout. To compare multiple populations, the Kruskal-Wallis non-parametric ANOVA was used with post hoc analysis using Dunn's test for multiple comparisons. The Mann-Whitney U test was used to compare two populations without consideration for paired samples. The ratio-paired t-test was used to compare two populations with consideration for paired samples and evidence of normality. P values in ANOVA analyses were corrected for multiple comparisons. A p value <0.05 was considered significant.

Exemplary viral receptor binding domains, including receptor binding motifs (RBM; shown in bold), are illustrated in Table 3 below. Receptor binding motif (RBM) is shown in bold.

TABLE 3

| | Genbank | |
|---|---|---|
| | Accession | |
| Coronavirus | Number | RBD Sequence (with RBM in bold) |
| HKU9 | NC_009021.1 | SYCTPPYSVLQDPPQPVVWRRYMLYDCVFDFTVVVDSLPTHQLQCYGVSPR |
| | | RLASMCYGSVTLDVMRINETHLNNLFNRVPDTFSLYNYALPDNFYGCLHAF |
| | | YLNSTAPYAVANRFPIKPGGRQSNSAFIDTVINAAHYSPFSYVYGLAVITL |
| | | KPAAGSKLVCPVAN |
| | | (SEQ ID NO: 38) |
| MERS | YP_00904720 | EAKPSGSVVEQAEGVECDFSPLLSGTPPQVYNFKRLVFTNCNYNLTKLLSL |
| | 4.1 | FSVNDFTCSQISPAAIASNCYSSLILDYFSYPLSMKSDLSVSSAGPISQFN |
| | | YKQSFSNPTCLILATVPHNLTTITKPLKYSYINKCSRFLSDDRTEVPQLVN |
| | | ANQYSPCVSIVPSTVWEDGDYYRKQLSPLEGGGWLVASGSTVAMTEQLQMG |
| | | FGITVQYGTDTNSVCPKLEFANDTKIASQL |
| | | (SEQ ID NO: 39) |
| Rs672/2006 | FJ588686.1 | RVSPTHEVIRFPNITNRCPFDKVFNASRFPNVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAKQDQGQYYYRSSRKTKLKPFERDLTSDENGVR |
| | | TLSTYDFYPNVPIEYQATRVVVLSFELLNAPATVCGPKL |
| | | (SEQ ID NO: 40) |
| Rp/Shaanxi2 | JX993987.1 | RVSPTQEVVRFPNITNRCPFDKVFNATRFPSVYAWERTKISDCVADYTVLY |
| 011 | | NSTSFSTFKCYGVSPSKLIDLCFTSVYADTFLIRSSEVRQVAPGETGVIAD |
| | | YNYKLPDDFTGCVIAWNTANQDQGQYYYRSSRKEKLKPFERDLSSDENGVY |
| | | TLSTYDFYPSVPLDYQATRVVVLSFELLNAPATVCGPKL |
| | | (SEQ ID NO: 41) |
| Cp/Yunnan2 | JX993988.1 | RVSPSTEVIRFPNITNRCPFDRVFNASRFPSVYAWERTKISDCVADYTVLY |
| 011 | | NSTSFSTFKCYGVSPSKLIDLCFTSVYADTFLIRFSEVRQIAPGETGVIAD |
| | | YNYKLPDEFTGCVIAWNTANQDRGQYYYRSSRKTKLKPFERDLSSDENGVR |
| | | TLSTYDFYPSVPLEYQATRVVVLSFELLNAPATVCGPKL |
| | | (SEQ ID NO: 42) |
| BtRf- | KJ473812.1 | RVSPVTEVVRFPNITNLCPFDKVFNATRFPSVYAWERTKISDCVADYTVFY |
| HeB2013 | | NSTSFSTENCYGVSPSKLIDLCFTSVYADTFLIRFSEVRQVAPGQTGVIAD |
| | | YNYKLPDDFTGCVIAWNTAKQDVGSYFYRSHRSSKLKPFERDLSSEENGVR |
| | | TLSTYDENQYVPLEYQATRVVVLSFELLNAPATVCGPKL |
| | | (SEQ ID NO: 43) |
| BtRf-SX2013 | KJ473813.1 | RVSPVTEVVRFPNITNLCPFDKVFNATRFPSVYAWERTKISDCVADYTVFY |
| | | NSTSFSTENCYGVSPSKLIDLCFTSVYADTFLIRFSEVRQVAPGQTGVIAD |
| | | YNYKLPDDFTGCVIAWNTAKQDVGSYFYRSHRSSKLKPFERDLSSEENGVR |
| | | TLSTYDFNQYVPLEYQATRVVVLSFELLNAPATVCGPKL |
| | | (SEQ ID NO: 44) |
| BtRs- | KJ473814.1 | RVTPTQEVVRFPNITNRCPFDRVENASRFPSVYAWERTKISDCVADYTVLY |
| HuB2013 | | NSTSFSTFKCYGVSPSKLIDLCFTSVYADTFLIRSSEVRQVAPGETGVIAD |
| | | YNYKLPDDFTGCVIAWNTAKQDTGYYYYRSHRKTKLKPFERDLSSDDGNGV |
| | | YTLSTYDENPNVPVAYQATRVVVLSFELLNAPATVCGPKL |
| | | (SEQ ID NO: 45) |
| BtRs- | KJ473815.1 | RVSPTQEVVRFPNITNRCPFDKVFNATRFPNVYAWERTKISDCVADYTVLY |
| GX2013 | | NSTSFSTFKCYGVSPSKLIDLCFTSVYADTFLIRSSEVRQVAPGETGVIAD |
| | | YNYKLPDDFTGCVIAWNTAKQDTGNYYYRSHRKTKLKPFERDLSSDDGNGV |
| | | YTLSTYDFNPNVPVAYQATRVVVLSFELLNAPATVCGPKL |
| | | (SEQ ID NO: 46) |
| BtRs- | KJ473816.1 | RVSPTREVVRFPNITNRCPFDSIFNASRFPSVYAWERTKISDCVADYTVLY |
| YN2013 | | NSTLFSTFKCYGVSPSKLIDLCFTSVYADTFLIRFSEVRQVAPGETGVIAD |
| | | YNYRLPDDFTGCVIAWNTANQDVGSYFYRSHRSTKLKPFERDLSSDENGVR |
| | | TLSTYDENPNVPLDYQATRVVVLSFELLNAPATVCGPKL |
| | | (SEQ ID NO: 47) |
| Longquan- | KF294457.1 | RVSPTQEVIRFPNITNRCPFDKVENVTRFPNVYAWERTKISDCVADYTVLY |
| 140 | | NSTSFSTFKCYGVSPSKLIDLCFTSVYADTFLIRSSEVRQVAPGETGVIAD |
| | | YNYKLPDDFTGCVIAWNTAKQDIGNYYYRSHRKTKLKPFERDLSSDDGNGV |
| | | YTLSTYDFNPNVPVAYQATRVVVLSFELLNAPATVCGPKL |
| | | (SEQ ID NO: 48) |
| YNLF_31C | KP886808.1 | RVAPVTEVVRFPNITNLCPFDKVFNATRFPSVYAWERTKISDCVADYTVFY |
| | | NSTSFSTENCYGVSPSKLIDLCFTSVYADTFLIRFSEVRQVAPGQTGVIAD |
| | | YNYKLPDDFTGCVIAWNTAKYDVGSYFYRSHRSSKLKPFERDLSSEENGAR |
| | | TLSTYDFNQNVPLEYQATRVVVLSFELLNAPATVCGPKL |
| | | (SEQ ID NO: 49) |
| YNLF_34C | KP886809.1 | RVAPVTEVVRFPNITNLCPFDKVFNATRFPSVYAWERTKISDCVADYTVFY |
| | | NSTSFSTENCYGVSPSKLIDLCFTSVYADTFLIRFSEVRQVAPGQTGVIAD |
| | | YNYKLPDDFTGCVIAWNTAKYDVGSYFYRSHRSSKLKPFERDLSSEENGAR |
| | | TLSTYDFNQNVPLEYQATRVVVLSFELLNAPATVCGPKL |
| | | (SEQ ID NO: 50) |
| As6526 | KY417142.1 | RVSPTQEVVRFPNITNRCPFDKVFNATRFPSVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAQQDKGQYYYRSSRKTKLKPFERDLSSDENGVR |
| | | TLSTYDFYPTVPIEYQATRVVVLSFELLNAPATVCGPKL |
| | | (SEQ ID NO: 51) |
| Rs4081 | KY417143.1 | RVSPTHEVVRFPNITNRCPFDKVFNASRFPNVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAKQDQGQYYYRSSRKTKLKPFERDLTSDENGVR |
| | | TLSTYDFYPNVPIEYQATRVVVLSFELLNAPATVCGPKL |
| | | (SEQ ID NO: 52) |
| Rs4237 | KY417147.1 | RVSPTQEVIRFPNITNRCPFDKVFNASRFPNVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAKQDQGQYYYRSSRKTKLKPFERDLSSDENGVR |
| | | TLSTYDFYPTVPIEYQATRVVVLSFELLNAPATVCGPKL |
| | | (SEQ ID NO: 53) |
| Rs4247 | KY417148.1 | RVSPTQEVIRFPNITNRCPFDKVENASRFPNVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAKQDTGHYYYRSHRKTKLKPFERDLSSDDGNGV |
| | | YTLSTYDFNPNVPVAYQATRVVVLSFELLNAPATVCGPKL |
| | | (SEQ ID NO: 54) |
| Rs4255 | KY417149.1 | RVSPTHEVIRFPNITNRCPFDKVENASRFPNVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAKQDQGQYYYRSSRKTKLKPFERDLSSDENGVR |
| | | TLSTYDFYPTVPIEYQATRVVVLSFELLNAPATVCGPKL |
| | | (SEQ ID NO: 55) |
| BtRI- | QDF43815.1 | RVSPTQEVVRFPNITNRCPFDKVENASRFPSVYAWERIKISDCVADYTVLY |
| BetaCoV/SC | | NSTSFSTFKCYGVSPSKLIDLCFTSVYADTFLIRSSEVRQVAPGETGVIAD |
| 2018 | | YNYKLPDDFTGCVIAWNTAKQDTGSYYYRSHRKTKLKPFERDLSSDDGNGV |
| | | YTLSTYDFNPNVPVAYQATRVVVLSFELLNAPATVCGPKL |
| | | (SEQ ID NO: 56) |
| BtRs- | MK211375.1 | RVSPTQEVVRFPNITNRCPFDKVENASRFPNVYAWERTKISDCVADYTVLY |
| BetaCoV/YN | | NSTSFSTFKCYGVSPSKLIDLCFTSVYADTFLIRSSEVRQVAPGETGVIAD |
| 2018A | | YNYKLPDDFTGCVIAWNTAKQDTGHYYYRSHRKTKLKPFERDLSSDDGNGV |
| | | YTLSTYDFNPNVPVAYQATRVVVLSFELLNAPATVCGPKL |
| | | (SEQ ID NO: 57) |
| BtRS- | MK211377.1 | RVSPTHEVIRFPNITNRCPFDKVFNASRFPNVYAWERTKISDCVADYTVLY |
| BetaCoV/YN | | NSTSFSTFKCYGVSPSKLIDLCFTSVYADTFLIRSSEVRQVAPGETGVIAD |
| 2018C | | YNYKLPDDFTGCVIAWNTAKQDQGQYYYRSSRKTKLKPFERDLTSDENGVR |
| | | TLSTYDFYPNVPIEYQATRVVVLSFELLNAPATVCGPKL |
| | | (SEQ ID NO: 58) |
| BtRs- | MK211378.1 | RVSPTHEVIRFPNITNRCPFDKVENASRFPNVYAWERTKISDCVADYTVLY |
| BetaCoV/YN | | NSTSFSTFKCYGVSPSKLIDLCFTSVYADTFLIRSSEVRQVAPGETGVIAD |
| 2018D | | YNYKLPDDFTGCVIAWNTAKQDQGQYYYRSSRKTKLKPFERDLTSDENGVR |
| | | TLSTYDFYPNVPIEYQATRVVVLSFELLNAPATVCGPKL |
| | | (SEQ ID NO: 59) |
| HKU3-1 | DQ022305.2 | RVSPTQEVIRFPNITNRCPFDKVENATREPNVYAWERTKISDCVADYTVLY |
| | | NSTSFSTFKCYGVSPSKLIDLCFTSVYADTFLIRSSEVRQVAPGETGVIAD |
| | | YNYKLPDDFTGCVIAWNTAKHDTGNYYYRSHRKTKLKPFERDLSSDDGNGV |
| | | YTLSTYDFNPNVPVAYQATRVVVLSFELLNAPATVCGPKL |
| | | (SEQ ID NO: 60) |
| HKU4 | NC_009019.1 | EASATGTFIEQPNATECDFSPMLTGVAPQVYNFKRLVFSNCNYNLTKLLSL |
| | | FAVDEFSCNGISPDSIARGCYSTLTVDYFAYPLSMKSYIRPGSAGNIPLYN |
| | | YKQSFANPTCRVMASVLANVTITKPHAYGYISKCSRLTGANQDVETPLYIN |
| | | PGEYSICRDFSPGGFSEDGQVFKRTLTQFEGGGLLIGVGTRVPMTDNLQMS |
| | | FIISVQYGTGTDSVCPMLDLGDSLTITNRL |
| | | (SEQ ID NO: 61) |
| HKU5 | NC_009020.1 | EASPRGEFIEQATTQECDFTPMLTGTPPPIYNFKRLVFTNCNYNLTKLLSL |
| | | FQVSEFSCHQVSPSSLATGCYSSLTVDYFAYSTDMSSYLQPGSAGAIVQEN |
| | | YKQDFSNPTCRVLATVPQNLTTITKPSNYAYLTECYKTSAYGKNYLYNAPG |
| | | AYTPCLSLASRGFSTKYQSHSDGELTTTGYIYPVTGNLQMAFIISVQYGTD |
| | | TNSVCPMQALRNDTSIEDKL |
| | | (SEQ ID NO: 62) |
| SARS-1 | ABD72970.1 | RVVPSGDVVRFPNITNLCPFGEVENATKFPSVYAWERKKISNCVADYSVLY |
| | | NSTFFSTFKCYGVSATKLNDLCFSNVYADSFVVKGDDVRQIAPGQTGVIAD |
| | | YNYKLPDDFMGCVLAWNTRNIDATSTGNYNYKYRYLRHGKLRPFERDISNV |
| | | PFSPDGKPCTPPALNCYWPLNDYGFYTTTGIGYQPYRVVVLSFELLNAPAT |
| | | VCGPKL |
| | | (SEQ ID NO: 63) |
| WIV1 | AGZ48828.1 | RVAPSKEVVRFPNITNLCPFGEVENATTFPSVYAWERKRISNCVADYSVLY |
| | | NSTSFSTFKCYGVSATKLNDLCFSNVYADSFVVKGDDVRQIAPGQTGVIAD |
| | | YNYKLPDDFTGCVLAWNTRNIDATQTGNYNYKYRSLRHGKLRPFERDISNV |
| | | PFSPDGKPCTPPAFNCYWPLNDYGFYITNGIGYQPYRVVVLSFELLNAPAT |
| | | VCGPKL |
| | | (SEQ ID NO: 64) |
| RaTG13 | QHR63300.2 | RVQPTDSIVRFPNITNLCPFGEVENATTFASVYAWNRKRISNCVADYSVLY |
| | | NSTSFSTFKCYGVSPTKLNDLCFTNVYADSFVITGDEVRQIAPGQTGKIAD |
| | | YNYKLPDDFTGCVIAWNSKHIDAKEGGNFNYLYRLFRKANLKPFERDISTE |
| | | IYQAGSKPCNGQTGLNCYYPLYRYGFYPTDGVGHQPYRVVVLSFELLNAPA |
| | | TVCGPKK |
| | | (SEQ ID NO: 65) |
| SHC014 | QJE50589.1 | RVAPSKEVVRFPNITNLCPFGEVENATTFPSVYAWERKRISNCVADYSVLY |
| | | NSTSFSTFKCYGVSATKLNDLCFSNVYADSFVVKGDDVRQIAPGQTGVIAD |
| | | YNYKLPDDFLGCVLAWNTNSKDSSTSGNYNYLYRWVRRSKLNPYERDLSND |
| | | IYSPGGQSCSAVGPNCYNPLRPYGFFTTAGVGHQPYRVVVLSFELLNAPAT |
| | | VCGPKLS |
| | | (SEQ ID NO: 66) |
| SARS-2 | MN975262.1 | RVQPTESIVRFPNITNLCPFGEVENATRFASVYAWNRKRISNCVADYSVLY |
| | | NSASFSTFKCYGVSPTKLNDLCFTNVYADSFVIRGDEVRQIAPGQTGKIAD |
| | | YNYKLPDDFTGCVIAWNSNNLDSKVGGNYNYLYRLERKSNLKPFERDISTE |
| | | IYQAGSTPCNGVEGFNCYFPLQSYGFQPTNGVGYQPYRVVVLSFELLHAPA |
| | | TVCGPKK |
| | | (SEQ ID NO: 67) |

TABLE 4

| SARSCoV2 Variants | |
|---|---|
| Being | |
| Monitored/Variants | |
| of Interest/Variants | |
| of Concern | Representative RBD Sequence (with RBM in bold) |
| Alpha (B.1.1.7, | RVQPTESIVRFPNITNLCPFGEVENATRFASVYAWNRKRISNCVADYSVLYNSASFST |
| Q.1Q.8) | FKCYGVSPTKLNDLCFTNVYADSFVIRGDEVRQIAPGQTGKIADYNYKLPDDFTGCVI |
| | AWNSNNLDSKVGGNYNYLYRLFRKSNLKPFERDISTEIYQAGSTPCNGVEGENCYFPL |
| | QSYGFQPTYGVGYQPYRVVVLSFELLHAPATVCGPKK (SEQ ID NO: 68) |
| Beta (B.1.351, | RVQPTESIVRFPNITNLCPFGEVENATRFASVYAWNRKRISNCVADYSVLYNSASFST |
| B.1.351.2, | FKCYGVSPTKLNDLCFTNVYADSFVIRGDEVRQIAPGQTGNIADYNYKLPDDFTGCVI |
| B.1.351.3) | AWNSNNLDSKVGGNYNYLYRLFRKSNLKPFERDISTEIYQAGSTPCNGVKGENCYFPL |
| | QSYGFQPTYGVGYQPYRVVVLSFELLHAPATVCGPKK (SEQ ID NO: 69) |
| Gamma (P.1, P.1.1, | RVQPTESIVRFPNITNLCPFGEVENATRFASVYAWNRKRISNCVADYSVLYNSASFST |
| P.1.2) | FKCYGVSPTKLNDLCFTNVYADSFVIRGDEVRQIAPGQTGTIADYNYKLPDDFTGCVI |
| | AWNSNNLDSKVGGNYNYLYRLFRKSNLKPFERDISTEIYQAGSTPCNGVKGFNCYFPL |
| | QSYGFQPTYGVGYQPYRVVVLSFELLHAPATVCGPKK (SEQ ID NO: 70) |
| Epsilon (B.1.427, | RVQPTESIVRFPNITNLCPFGEVENATRFASVYAWNRKRISNCVADYSVLYNSASFST |
| B.1.429) | FKCYGVSPTKLNDLCFTNVYADSFVIRGDEVRQIAPGQTGKIADYNYKLPDDFTGCVI |
| | AWNSNNLDSKVGGNYNYRYRLFRKSNLKPFERDISTEIYQAGSTPCNGVEGENCYFPL |
| | QSYGFQPTNGVGYQPYRVVVLSFELLHAPATVCGPKK (SEQ ID NO: 71) |
| Eta (B.1.525) | RVQPTESIVRFPNITNLCPFGEVENATRFASVYAWNRKRISNCVADYSVLYNSASFST |
| | FKCYGVSPTKLNDLCFTNVYADSFVIRGDEVRQIAPGQTGKIADYNYKLPDDFTGCVI |
| | AWNSKNLDSKVGGNYNYLFRLFRKSNLKPFERDISTEIYQAGSTPCNGVKGENCYFPL |
| | QSYGFQPTNGVGYQPYRVVVLSFELLHAPATVCGPKK (SEQ ID NO: 72) |
| lota (B.1.526) | RVQPTESIVRFPNITNLCPFGEVENATRFASVYAWNRKRISNCVADYSVLYNSASFST |
| | FKCYGVSPTKLNDLCFTNVYADSFVIRGDEVRQIAPGQTGKIADYNYKLPDDFTGCVI |
| | AWNSNNLDSKVGGNYNYLYRLFRKSNLKPFERDISTEIYQAGSTPCNGVKGFNCYFPL |
| | QSYGFQPTNGVGYQPYRVVVLSFELLHAPATVCGPKK (SEQ ID NO: 73) |
| Kappa (B.1.617.1) | RVQPTESIVRFPNITNLCPFGEVENATRFASVYAWNRKRISNCVADYSVLYNSASFST |
| | FKCYGVSPTKLNDLCFTNVYADSFVIRGDEVRQIAPGQTGKIADYNYKLPDDFTGCVI |
| | AWNSNNLDSKVGGNYNYRYRLFRKSNLKPFERDISTEIYQAGSTPCNGVQGFNCYFPL |
| | QSYGFQPTNGVGYQPYRVVVLSFELLHAPATVCGPKK (SEQ ID NO: 74) |
| Zeta (P.2) | RVQPTESIVRFPNITNLCPFGEVENATRFASVYAWNRKRISNCVADYSVLYNSASFST |
| | FKCYGVSPTKLNDLCFTNVYADSFVIRGDEVRQIAPGQTGKIADYNYKLPDDFTGCVI |
| | AWNSNNLDSKVGGNYNYLYRLFRKSNLKPFERDISTEIYQAGSTPCNGVKGENCYFPL |
| | QSYGFQPTNGVGYQPYRVVVLSFELLHAPATVCGPKK (SEQ ID NO: 75) |
| Mu (B.1.621, | RVQPTESIVRFPNITNLCPFGEVENATRFASVYAWNRKRISNCVADYSVLYNSASFST |
| B.1.621.1) | FKCYGVSPTKLNDLCFTNVYADSFVIRGDEVRQIAPGQTGKIADYNYKLPDDFTGCVI |
| | AWNSNNLDSKVGGNYNYLYRLFRKSNLKPFERDISTEIYQAGSTPCNGVKGENCYFPL |
| | QSYGFQPTYGVGYQPYRVVVLSFELLHAPATVCGPKK (SEQ ID NO: 76) |
| Delta (B.1.617.2, | RVQPTESIVRFPNITNLCPFGEVENATRFASVYAWNRKRISNCVADYSVLYNSASFST |
| AY.1 sublineages) | FKCYGVSPTKLNDLCFTNVYADSFVIRGDEVRQIAPGQTGKIADYNYKLPDDFTGCVI |
| | AWNSNNLDSKVGGNYNYRYRLFRKSNLKPFERDISTEIYQAGSKPCNGVEGENCYFPL |
| | QSYGFQPTNGVGYQPYRVVVLSFELLHAPATVCGPKK (SEQ ID NO: 77) |

Exemplary immunogens generated and disclosed herein are illustrated in Table 5 below.

TABLE 5

| Immunogen | Amino Acid Sequence |
|---|---|
| SARS-2^{hg} | RVQPTESIVRFPNITNLCPFGEVEN |
| trimer | ATRFASVYAWNRSRISNCVADYSVL |
| | YNSSSFSTFKCYGVNATKLNDLCFT |
| | NVYADSFVIRGDEVRQIAPGQTGKI |
| | ADYNYKLPDDFTGCVIAWNSNNNDS |
| | KVGGNYNYLYRLFRKSNLKPFERDN |
| | STEIYQAGSTPCNGVEGENCYFPLQ |
| | SYGFQPTNGVGYQPYRVVVLSFELN |
| | HSPATVCGPKKGAGSSGSGRMKQIE |
| | DKIENITSKIYNITNEIARIKKCCG |
| | NRTSGGLEVLFQGPGSSHHHHHHHH |
| | (SEQ ID NO: 78) |
| SARS-2 RBM^{hg} | RVQPTESIVRFPNITNLCPFGEVEN |
| trimer | ATRFASVYAWNRKRISNCVADYSVL |
| | YNSASFSTFKCYGVSPTKLNDLCFT |
| | NVYADSFVIRGDEVRQIAPGQTGKI |
| | ADYNYKLPDDFTGCVIAWNSNNLDS |
| | KVGGNYSYLYRLERKSNLKPFERDI |
| | STEIYQNGSTPCNGVEGENCYFPLQ |
| | NYSFQPTNGTGYQPYRVVVLSFELL |
| | HAPATVCGPKKGAGSSGSGRMKQIE |
| | DKIENITSKIYNITNEIARIKKCCG |
| | NRTSGGLEVLFQGPGSSHHHHHHHH |
| | (SEQ ID NO: 79) |
| rsWIV1^{hg} | RVAPSKEVVRFPNITNLCPFWEVEN |
| trimer | ATTFPSVYAWNRSRISNCVADYSVL |
| | YNSTSFSTFACYGVNATALNDLCFS |
| | NVYADSFVVKGDDVRQIAPGQTGVI |
| | ADYNYKLPDDERGCVLAWNSNNNDS |
| | KVGGNYNYLYRLFRKSNLKPFERDN |
| | STEIYQAGSTPCNGVEGENCYFPLQ |
| | SYGFQPTNGVGYQPYRVVVLSFELN |
| | NSPATVCGPKLGAGSSGSGRMKQIE |
| | DKIENITSKIYNITNEIARIKKCCG |
| | NRTSGGLEVLFQGPGSSHHHHHHHH |
| | (SEQ ID NO: 80) |
| rsSARS-1^{hg} | RVVPSGDVVRFPNITNLCDFGEVEN |
| trimer | ATKFPSVYAWNRSKISNCVADYSVL |
| | YNSTFFSTFACYRVNATKLNDLCFS |
| | NVYADSFVVKGDDVRQIAPGQTGVI |
| | ADYNYKLPDDFKGCVLAWNSNNNDS |
| | KVGGNYNYLYRLFRKSNLKPFERDN |
| | STEIYQAGSTPCNGVEGENCYFPLQ |
| | SYGFQPTNGVGYQPYRVVVLSFELN |
| | NSPATVCGPKLGAGSSGSGRMKQIE |
| | DKIENITSKIYNITNEIARIKKCCG |
| | NRTSGGLEVLFQGPGSSHHHHHHHH |
| | (SEQ ID NO: 81) |

For all sequences in Table 5, a hgGCN4^{cys} tag is present and is represented by the amino acid sequence RMKQIEDKIENITSKIYNITNEIARIKKCCGNRT (SEQ ID NO: 82); an HRV 3C protease cleavage site is present and represented by the amino acid sequence LEVLFQGP (SEQ ID NO: 83); and an 8×His affinity tag is present and represented by the amino acid sequence HHHHHHHH (SEQ ID NO: 84). For all sequences in Table 5, linker sequences are present between: the immunogen and the hgGCN4^{cys} tag (e.g., GAGSSGSG (SEQ ID NO: 85)); the hgGCN4^{cys} tag and the HRV 3C protease cleavage site (e.g., SGG (SEQ ID NO: 86)); and the HRV 3C protease cleavage site and the 8×His affinity tag (e.g., GSS (SEQ ID NO: 87)). The linker sequences are represented in Table 5 by the underlined amino acids.

III. Immunization with Resurfaced Receptor Binding Domains Conjugated to Ferritin Nanoparticles Results in Broad Neutralization with a Large Scaffold-Directed Antibody Response

In this study, we conjugated resurfaced RBDs to ferritin nanoparticles and then immunized wild-type mice in a homologous prime-boost format with these 24-mer antigens. In characterizing the resulting serum immune response, we found that the serum antibody response reacted towards all components of the vaccine construct. This included the ferritin nanoparticle, which could impact the extent to which repeated boosting with ferritin nanoparticle-conjugated immunogens is feasible. Pseudovirus neutralization was highest against SARS-2 across all cohorts, indicating that these immunization regimens may elicit neutralizing antibodies that target the SARS-2 RBM and that therefore lack breadth against related sarbecoviruses due to RBM antigenic differences.

### Results

### Nanoparticle Production

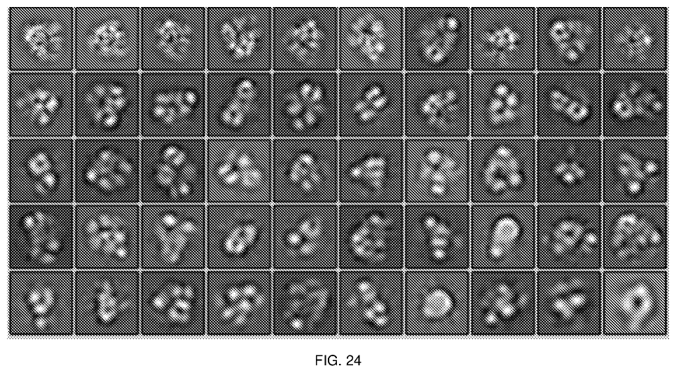

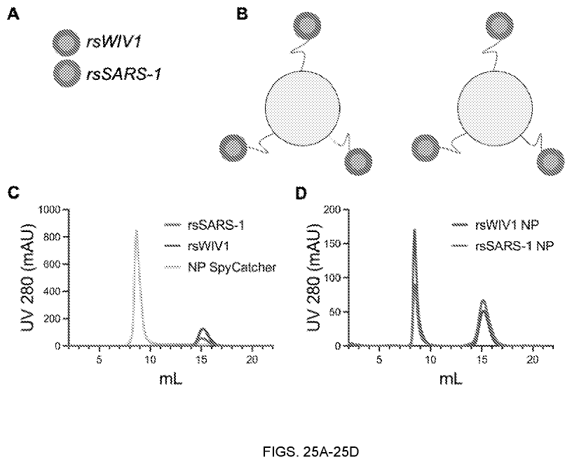

The previous examples described the process of grafting the SARS-2 receptor binding motif (RBM) onto heterologous coronavirus scaffolds, specifically the SARS-1 and WIV1 receptor binding domains (RBDs). This resulted in the resurfaced SARS-1 (rsSARS-1) and resurfaced WIV1 (rsWIV1) RBD constructs (FIG. 25A). These immunogens can be leveraged to elicit antibody responses to the SARS-2 RBM as well as the RBDs from the heterologous coronaviruses used as scaffolds, which may confer broad neutralization of sarbecoviruses.

We generated multimerized versions of these resurfaced RBDs using the SpyTag/SpyCatcher covalent attachment system (Zakeri et al., Proc Natl Acad Sci USA 109, E690-697 (2012)) to decorate a 24-mer ferritin nanoparticle with a single RBD, as previously described (FIG. 25B). To do this, we first expressed the SpyCatcher ferritin nanoparticles and RBDs with SpyTag separately (FIG. 25C). We then combined a 1.2 molar excess of each RBD with the SpyCatcher ferritin nanoparticle overnight and repeated size exclusion chromatography to separate the conjugated nanoparticle from the excess RBD (FIG. 25D).

### Testing Nanoparticle Constructs in a Mouse Model

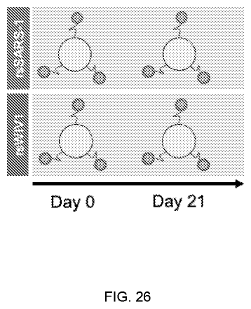

To evaluate the immunogenicity of the conjugated nanoparticles, we immunized C57BL/6 mice in a homologous prime-boost format. One cohort received the rsSARS-1 nanoparticle (“rsSARS-1 cohort”), while the other cohort received the rsWIV1 nanoparticle (“rsWIV1 cohort”); both cohorts contained N=5 mice (FIG. 26). Each mouse received 20 μg of protein adjuvanted with Sigma Adjuvant via intraperitoneal injection at day 0 and day 21.

### Characterizing Serum Reactivity

At day 35, serum samples were collected from each mouse for characterization of immunogenicity. We first tested serum titers against a variety of coronavirus proteins, including the wild-type SARS-2 RBD, the SARS-2 spike, the SARS-1 RBD, and the WIV1 RBD. We also measured titers against the SARS-2 RBM^{hg} construct, which has engineered glycans across the RBM. Comparing titers against the SARS-2 RBM^{hg} construct and the wild-type SARS-2 RBD can indicate whether the majority of SARS-2 RBD-directed serum antibodies target the SARS-2 RBM, as these antibodies would be less likely to bind to the SARS-2 RBM^{hg} construct.

In the rsSARS-1 cohort, there were significantly higher titers against the SARS-1 and WIV1 RBDs compared to the full-length SARS-2 spike ectodomain (FIG. 27A). There were no other statistically significant differences in titers, including between the wild-type SARS-2 RBD and the SARS-2 RBM^{hg} construct. In the rsWIV1 cohort, there were no statistically significant differences in titers, but a similar pattern of reactivity to the rsSARS-1 cohort was observed with lower titers against the SARS-2 spike in comparison to all of the tested RBDs (FIG. 27B).

We also wanted to evaluate reactivity against additional sarbecovirus RBDs to get a sense of the breadth of the antibody responses elicited by these immunogens. Reactivity against the Omicron BA. 1 RBD, the RaTG13 RBD, and the SHC014 RBD was markedly lower across both cohorts, within the ˜10³-10⁴ range versus the ˜10⁴-105 range of titers against the SARS-2, SARS-1, and WIV1 RBDs (FIG. 27C). This indicates that while there are some cross-reactive antibody responses elicited by these immunogens, much of the elicited serum antibody response targets antigens included in the original immunization.

Given this observation, we also wanted to measure the titers against the SpyCatcher ferritin nanoparticle. This was found to be within the ˜10⁴-105 range across both the rsSARS-1 and rsWIV1 cohorts (FIG. 27D).

### Characterizing Serum Neutralization

We used a pseudovirus neutralization assay to quantify neutralization of SARS-2, SARS-1, and WIV1 across both cohorts. Within the rsSARS-1 cohort, there was no significant difference in neutralization between the three pseudoviruses (FIG. 28A). However, neutralization of SARS-1 and WIV1 was approximately an order of magnitude less than neutralization of SARS-2. This pattern was observed within the rsWIV1 cohort as well, with differences in neutralization of SARS-1 and WIV1 in comparison to SARS-2 reaching statistical significance (FIG. 28B). This may indicate that the majority of the neutralizing antibody response targets epitopes in SARS-2 that are not conserved across SARS-1 and WIV1. The SARS-2 RBM varies considerably in comparison to the SARS-1 (49.3% amino acid identity) and WIV1 (52.1% amino acid identity) RBMs, whereas the non-RBM portions of the SARS-1 (85.0% amino acid identity) and WIV1 (87.1% amino acid identity) RBDs are more similar to SARS-2. This may indicate that the majority of the neutralizing antibody response elicited by these immunization regimens targets the SARS-2 RBM.

The above-described results indicate that immunizing C57BL/6 mice with the rsSARS-1 and rsWIV1 RBD conjugated to ferritin nanoparticles resulted in a predominantly SARS-2-neutralizing response, with breadth expanding to include neutralization of SARS-1 and WIV1 pseudoviruses as well. This may indicate that immunizing naïve mice with these constructs results in a serum response in which a large extent of the neutralizing antibody response targets the SARS-2 RBM, which shares limited homology with the RBMs of related sarbecoviruses like SARS-1 and WIV1

In addition to the RBD-directed antibody response elicited by these immunogens, there is also a significant SpyCatcher nanoparticle-directed serum antibody response. In our study, SpyCatcher nanoparticle ELISA titers were comparable to titers against the SARS-2, SARS-1, and WIV1 RBDs. These high titers indicate that boosting with nanoparticle conjugated immunogens repeatedly could result in a considerable nanoparticle-directed antibody response, particularly in a scenario where nanoparticle-directed memory B cells exist but the immune system is naïve to the antigen conjugated to the nanoparticle. This immune imprinting phenomenon has previously been highlighted with respect to seasonal influenza vaccine antibody responses, which can be preferentially directed towards non-neutralizing, conserved epitopes. Boosting with SpyCatcher nanoparticle-conjugated immunogens in the context of prior SpyCatcher nanoparticle imprinting might be similarly disadvantageous, resulting in an antibody response that preferentially targets conserved epitopes on the conserved SpyCatcher nanoparticle rather than novel epitopes on the conjugated immunogen.

These results provide useful immunogenicity profiling data that can inform future design efforts across a range of viral families. In particular, these findings raise the concern that repeated immunization with SpyCatcher nanoparticle-conjugated immunogens could result in a substantial SpyCatcher nanoparticle-directed antibody response. The effect of this off-target antibody response with respect to vaccine efficacy merits further investigation.

The above results were obtained using the following materials and methods.

### Materials and Methods

### Immunogen and Coronavirus Protein Expression and Purification

SARS-2 RBM boundaries from residues 437-507 were used to construct the rsSARS-1 and rsWIV1 RBDs. Genbank accession numbers for the RBDs expressed in this study are as follows: SARS-2 RBD (Genbank MN975262.1), SARS-1 RBD (Genbank ABD72970.1), WIV1 RBD (Genbank AGZ48828.1), RaTG13 RBD (Genbank QHR63300.2), SHC014 RBD (Genbank QJE50589.1). The SARS-2 spike plasmid contained a non-cleavable Foldon trimerization domain and C-terminal HRV 3C-cleavable 6×His and 2× Strep II tags. For use in ELISAs, all RBDs were codon optimized and cloned into the pVRC expression vector containing a C-terminal HRV 3C-cleavable 8×His tag and SBP tag. Resurfaced RBDs for use in immunogens were cloned into the pVRC expression vector containing a C-terminal HRV 3C-cleavable 8×His tag and SpyTag (B. Zakeri et al., Proc Natl Acad Sci USA 109, E690-697 (2012)). The SpyCatcher-ferritin nanoparticle fusion was designed based on previously published constructs (B. Zakeri et al., Proc Natl Acad Sci USA 109, E690-697 (2012); M. Kanekiyo et al., Nature 499, 102-106 (2013)) and was cloned into pVRC with a C-terminal 8×His tag.

All constructs were expressed in Expi293F cells (ThermoFisher) using Expifectamine transfection reagents according to the manufacturer's protocol. Transfection supernatants were harvested after 5-7 days and purified using Cobalt-TALON resin (Takara) for immobilized affinity chromatography. Following elution, proteins underwent additional purification by size-exclusion chromatography using a Superdex 200 Increase 10/300 GL column (Cytiva).

### Nanoparticle Conjugation

SpyCatcher nanoparticles were incubated overnight at 4° C. with rotation with a 1.2-molar excess of resurfaced RBDs with SpyTag. Afterwards, the conjugation reaction was purified by size-exclusion chromatography using a Superdex 200 Increase 10/300 GL column (Cytiva) to separate the conjugated nanoparticle from excess resurfaced RBD. Fractions corresponding to the conjugated nanoparticle peak were concentrated and used for immunizations.

### Immunizations

All immunization experiments were conducted with institutional IACUC approval (MGH protocol 2014N000252). Immunizations were performed using C57BL/6 female mice (Charles River Laboratories) aged 6-10 weeks. Immunizations occurred at days 0 and 21, and each mouse received an intraperitoneal injection containing 20 μg of protein adjuvanted with 50% w/v Sigma Adjuvant System in 100 μL of inoculum. Serum characterization was performed using day 35 samples.

### Serum ELISAs

Serum ELISAs were performed as described above. Briefly, coating occurred overnight at 4° C. using 100 μL of protein at 5 μg/mL. Blocking was performed for 60 minutes at room temperature using 150 μL of 1% BSA in PBS-Tween. Diluted mouse serum was used as the primary antibody, with a starting dilution of 1:40 followed by a serial 5-fold dilution. Primary incubation occurred for 90 minutes at room temperature. After washing, 150 μL of HRP-conjugated anti-mouse IgG (Abcam) was added at a concentration of 1:20,000 in PBS. Secondary incubation occurred for 60 minutes at room temperature. Plates were then washed and developed for 30 minutes at room temperature with 150 μL of 1× ABTS (ThermoFisher). Development was stopped with 100 μL of 1% SDS, and plates were read for absorbance at 405 nm on a SpectraMaxiD3 plate reader (Molecular Devices).

### Pseudovirus Neutralization Assay

Serum pseudovirus neutralization assays were performed according to methods described herein. Briefly, transient transfection of 293T cells was used to produce pseudotyped lentiviral particles. Pseudoviruses were tittered using 293T-ACE2 cells (M. J. Moore et al., J Virol 78, 10628-10635 (2004)) and the HIV-1 p24CA antigen capture assay (Leidos Biomedical Research, Inc.). Pseudovirus neutralization assays were performed with a Tecan Fluent Automated Workstation using 384-well plates (Grenier). Mouse sera underwent an initial 1:3 dilution, with subsequent 3-fold dilutions. 20 μL each of sera and pseudovirus (125 infectious units) was added to each well. Incubation occurred for 60 minutes at room temperature. Then, 10,000 293T-ACE2 cells (M. J. Moore et al., J Virol 78, 10628-10635 (2004)) as well as 15 μg/mL polybrene was added. Incubation occurred at 37° C. for 60-72 hours. Following cell lysis as previously described (E. Siebring-van Olst et al., J Biomol Screen 18, 453-461 (2013)), luciferase expression was quantified using a Spectramax L luminometer (Molecular Devices).

To calculate neutralization percentage, background luminescence from cells-only wells was subtracted from each sample and then divided by the luminescence of wells that contained only cells and virus, without any mouse sera. Nonlinear regressions were fit using GraphPad Prism (version 9) to calculate IC50 values for samples that attained at least 80% neutralization (as measured with maximum serum concentration). NT50 values were then calculated by taking the reciprocal of the IC50 values.

### IV. Cross-Reactive SARS-CoV-2 Epitope Targeted Across Donors Informs Immunogen Design

The data described below demonstrate how immune-focusing to a conserved epitope targeted by human cross-reactive antibodies guides pan-sarbecovirus vaccine development, providing a template for identifying such epitopes and translating to immunogen design.

### Results

### An Engineered Immunogen for Epitope Targeting

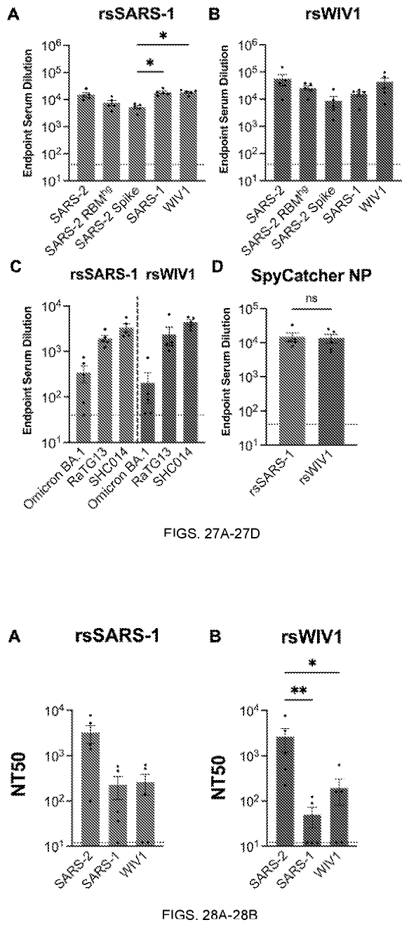

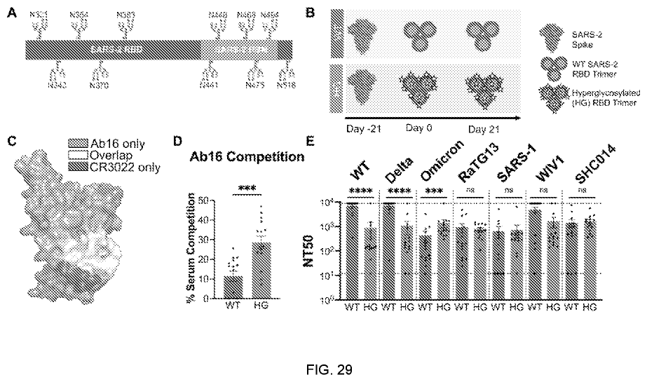

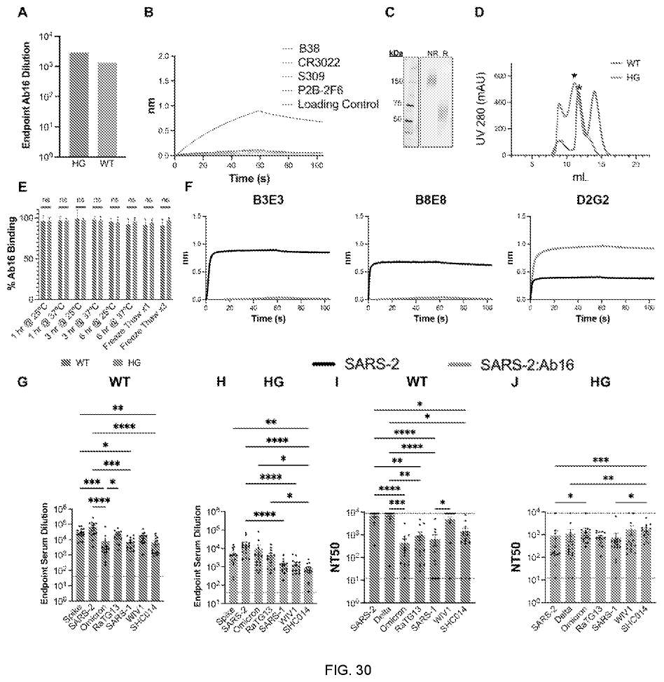

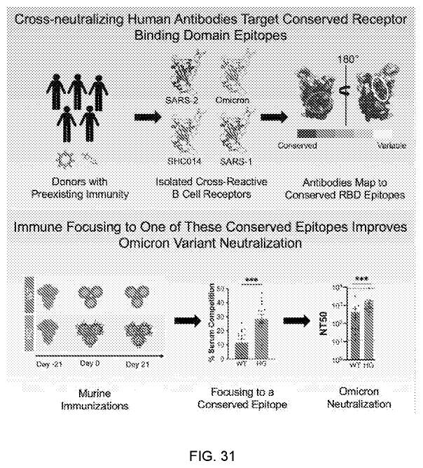

Based on these observations across human donors, we used a structure-guided approach to design an immunogen to focus antibody responses to the class 4 epitope. Using glycan engineering, we developed a hyperglycosylated immunogen that selectively exposed this epitope while occluding all other epitopes (FIG. 29A, 30A-B); this builds upon our previous hyperglycosylated immunogens to direct immune responses to conserved epitopes. This class 4-focusing immunogen has 11 engineered putative N-linked glycosylation sites; 9 are novel and two are native to SARS-2. We made trimeric versions of this immunogen using a non-immunogenic, hyperglycosylated GCN4 trimerization tag to increase valency and for subsequent testing in vivo (FIG. 30C-F). Two murine cohorts were primed with the SARS-2 spike to approximate pre-existing vaccine or infection-elicited immunity to SARS-2 followed by boosting with trimeric wild-type SARS-2 RBD (“WT Cohort”) or the hyperglycosylated immunogen (“HG Cohort”) (FIG. 29B).

### Assessing Immunogenicity and Focusing

We assayed binding of sera 14 days after the second boost against coronavirus proteins by ELISA. Both cohorts had similar reactivity to all sarbecovirus RBDs tested, but the WT cohort showed a significant drop-off in reactivity against the Omicron RBD versus the SARS-2 RBD while the HG cohort did not (FIG. 30G-H). To assess immune focusing to the class 4 epitope, we assayed competition of the murine class 4 antibody Ab16, which competes with CR3022 but has broad neutralization activity across SARS-2 variants and other sarbecoviruses. Ab16 competes with CR3022 and the class 4 donor antibodies (FIG. 29C, 30F), and the Ab16 footprint was previously structurally characterized; Ab16 was also used to confirm the conformational integrity of the HG immunogen (FIG. 30A). The HG cohort had significant increase in serum competition with Ab16, with mean serum competition of ˜2.5× relative to WT (FIG. 29D). This indicates that boosting with the hyperglycosylated immunogen immune focuses to the class 4 epitope.

### Immune Focusing Improves Cross-Neutralization

We assessed serum pseudovirus neutralization of wild-type SARS-2, Delta and Omicron variants, and related sarbecoviruses RaTG13, SARS-1, WIV1, and SHC014. Sera from all cohorts had broad neutralization with NT50s ranging from ˜10³-10⁴ against wild-type SARS-2 to ˜10²-10³ against SARS-1 and WIV1 (FIG. 29E). Neutralization of wild-type SARS-2 and the closely related Delta variant was significantly lower in the HG cohort, however neutralization of Omicron was significantly improved in the HG Cohort (FIG. 29E). Neutralization of related sarbecoviruses RaTG13, SARS-1, WIV1, and SHC014 showed no significant differences between the WT and HG cohorts (FIG. 29E). This is consistent with the fact that there is no significant decrease in the HG cohort between neutralization of wild-type SARS-2 and all other coronaviruses tested, while there are significant decreases in the WT cohort (FIGS. 30I-J). Because serum antibody responses from the HG cohort significantly competed with Ab16 (class 4 epitope), relative to the WT Cohort (FIG. 29D), this indicates that the observed immune focusing elicited by this first-generation immunogen may also neutralize future antigenically distinct SARS-2 variants. While these data demonstrate a proof-of-principle that focusing SARS-2 serum antibody responses to a broadly neutralizing epitope can improve breadth, the lack of improved serum neutralization across other sarbecoviruses (FIGS. 30I-J) indicates that further iterative design cycles would likely be required.

The above-described results indicate that human subjects generate cross-reactive antibodies targeting conserved epitopes, and that these antibodies are genetically diverse. In mice, selectively boosting responses to one of these conserved epitopes using an engineered immunogen improved Omicron variant neutralization, in the context of preexisting immunity. These data provide evidence for a pan-sarbecovirus vaccine design by demonstrating that prior knowledge of the exact variant or emerging sarbecovirus may not be required to elicit a broadly neutralizing antibody response.

Thus, eliciting broadly cross-reactive antibody responses via vaccination, in combination with developing broadly protective therapeutic antibodies, may help reduce the likelihood that ongoing SARS-2 evolution and potential emerging sarbecoviruses will escape both existing protective immunity and available treatments.

The above results were obtained using the following materials and methods.

### Materials and Methods

### Mice

All immunizations were performed using female C57BL/6 mice (Charles River Laboratories, strain 027) aged 6-10 weeks. Immunization experiments were conducted with institutional IACUC approval (MGH protocol 2014N000252).

### Cell Lines

Expi293F cells (Thermo Fisher Cat #A14527; RRID: CVCL_D615) cells were cultured in accordance with the manufacturer's instructions. Human ACE2 expressing HEK 293T cells (Moore et al., (2004). J Virol 78, 10628-10635. 10.1128/JVI.78.19.10628-10635.2004) were a gift from Nir Hacohen and Michael Farzan and were cultured in Dulbecco's Modified Eagle Medium (ThermoFisher) with 2% fetal bovine serum (Peak Serum FBS) and 1% penicillin-streptomycin at 10,000 U/mL (Gibco). A549-Ace2 cells (BEI Resources, Cat #NR-53821) were cultured in in D10+ media (DMEM (Corning) supplemented with HEPES (Corning), 1× Penicillin 100 IU/mL/Streptomycin 100 μg/mL (Corning), 1× Glutamine (Glutamax, ThermoFisher Scientific), and 10% Fetal Bovine serum (FBS; Sigma).

### Immunogen and Coronavirus Protein Expression and Purification

Coronavirus receptor binding domains (RBDs) were based on the following Genbank sequences: SARS-2 RBD (Genbank MN975262.1), SARS-1 RBD (Genbank ABD72970.1), WIV1 RBD (Genbank AGZ48828.1), RaTG13 RBD (Genbank QHR63300.2), SHC014 RBD (Genbank QJE50589.1). All constructs were purchased as gblocks following codon optimization using Integrated DNA Technologies. Genes were cloned into pVRC and sequence confirmed via Genewiz. Monomeric constructs used for ELISA coating and single B cell sorting included C-terminal HRV 3C-cleavable 8×His and Avi tags, and trimeric constructs included a C-terminal HRV 3C-cleavable 8×His tag and a hyperglycosylated GCN4 tag with two engineered C-terminal cystines, the lattermost tag being derived from a previously published hyperglycosylated GCN4 tag (Sliepen et al., J Biol Chem 290, 7436-7442 (2015)). The spike plasmid was provided courtesy of Dr. Jason Mclellan at the University of Texas, Austin, containing C-terminal HRV 3C cleavable 6×His and 2× Strep II tags and a non-cleavable foldon trimerization domain (Wrapp, et al., Science 367, 1260-1263. (2020)).

Proteins were expressed in Expi293F cells (ThermoFisher) after transfection using Expifectamine per manufacturer's protocol. After 7 days, transfections were harvested and centrifuged for clarification. Immobilized metal-affinity chromatography using Cobalt-TALON resin (Takara) was performed via the 8×His tag. Proteins were eluted with 350 mM imidazole, concentrated, and passed over a size exclusion column (Superdex 200 Increase 10/300 GL, GE Healthcare) in PBS (Corning). Affinity tags were cleaved by HRV 3C protease (ThermoScientific) and purified protein was isolated by orthogonal purification using Cobalt-TALON resin to remove the His-tagged HRV 3C protease, cleaved tag, and uncleaved protein.

### IgG Expression and Purification

Variable heavy and light chain genes were synthesized as gBlocks from Integrated DNA Technologies and cloned into pVRC plasmids containing appropriate constant domains (Schmidt, et al., Cell Rep 13, 2842-2850. (2015); Schmidt, et al., Cell 161, 1026-1034. (2015)). Fabs and IgGs were purified and transfected using the same protocol for immunogens and coating proteins. The heavy chain plasmid included a HRV 3C-cleavable 8×His tag, and the IgG heavy chain vector included HRV 3C-cleavable 8×His tags.

### Serum and Recombinant IgG ELISAs

Serum ELISAs were done using Corning clear flat-bottom 96-well high binding microplates. Plates were coated with protein a concentration of 2.5 μg/mL (in PBS) at a working volume of 100 μL, then incubated overnight (>8 hours) at 4° C. Following incubation, coating solution was decanted, and all wells were blocked with 150 μL of 1% BSA in PBS with 1% Tween for 120 minutes at ambient temperature. After blocking, sera diluted 40-fold or IgGs at a specified concentration were prepared as primary antibody solution, then serially diluted 5-fold in tubes. For serum ELISAs, CR3022 IgG was also serially diluted (5-fold) from a 5 μg/mL starting concentration, to serve as a control curve. 40 μL of primary antibody solution was then added to each well, and plates were incubated at ambient temperature for 90 minutes. After incubation, plates were washed three times in PBS/Tween solution.

The secondary antibody solution consisted of Abcam HRP-conjugated rabbit anti-mouse IgG antibody (for mouse serum ELISAs) or Abcam HRP-conjugated goat anti-human IgG antibody (for human IgG ELISAs) diluted 1:20,000 in PBS. 150 μL of the resulting solution was added to each well before incubating for one hour at ambient temperature. Plates were again washed three times with PBS/Tween solution. Following manufacturer (ThermoFisher) protocol, 150 μL of 1× ABTS development solution was added to each well for color development, which was arrested after 30 minutes with 100 μL of 0.1% sodium azide. Plates were read using a SpectraMaxiD3 plate reader (Molecular Devices) for absorbance at 405 nm.

### Competition Biolayer Interferometry

Antibody competition with D2G2 (a representative of the group of antibodies that does not target a class 4 epitope) was assessed via biolayer interferometry (BLI). SARS-2 RBD at 8 UM was complexed with at least a 5-molar excess of Fab for 30 minutes. Binding of the complex to D2G2 Fab was measured using a BLItz (ForteBio). Ni-NTA sensors (Sartorius) were used with D2G2 immobilized and complexes as the analytes. Absorbance was recorded and used to qualitatively assess whether D2G2 was able to bind to the complex. All reagents were diluted in 1× Kinetics Buffer (ForteBio).

Antibody competition with Ab16 (a class 4 antibody) was also assessed via BLI. SARS-2 RBD at 8 UM was complexed with at least a 5-molar excess Ab16 Fab for 30 minutes, and then binding of the complex to the B3E3, B8E8, and D2G2 Fabs was measured using a BLItz (ForteBio) and compared to binding of SARS-2 RBD at 8 μM without Ab16 to qualitatively determine whether each Fab was able to bind the SARS-2 RBD:Ab16 complex. FAB2G sensors were used with B3E3, B8E8, or D2G2 immobilized and SARS-2 RBD:Ab16 complexes or SARS-2 RBD as the analytes. All reagents were diluted in 1× Kinetics Buffer (ForteBio).

### Immunizations

Female C57BL/6 mice (Charles River Laboratories) aged 6-10 weeks were used for all immunizations. Mice were immunized by the intraperitoneal route, introducing 20 μg of protein adjuvanted with 50% w/v Sigma adjuvant per 100 μL of inoculum. Priming occurred on day-21, boosts occurred at days 0 and 21, and serum was collected on day 35 from all cohorts. Two separate replicates of the immunization experiments were performed, the first with N=5 mice per cohort and the second with N=10 mice per cohort. All immunizations were approved by institutional IACUC (MGH protocol 2014N000252).

### Pseudovirus Neutralization Assay

Monoclonal IgGs and mouse sera were assayed against wild-type SARS-2, Omicron variant SARS-2, Delta variant SARS-2, SARS-1, WIV1, RaTG13, and SHC014 pseudotyped lentiviral particles expressing spike proteins described previously (Garcia-Beltran, et al., Cell 184, 476-488 (2021)). Lentiviral particles were generated via transient transfection of 293T cells, with viral supernatant titers assesed via flow cytometry of 293T-ACE2 cells (Moore, et al., J Virol 78, 10628-10635. (2004)) and the HIV-1 p24CA antigen capture assay (Leidos Biomedical Research, Inc.). All assays were performed in a 384-well format (plates from Grenier) using a Tecan Fluent Automated Workstation.

Mouse serum samples started at an initial 1:3 dilution followed by six subsequent serial 3-fold dilutions. Monoclonal IgGs started at an initial specified concentration and then were subsequently diluted 3-fold as well. Each plate well contained 20 μL of sera and 20 μL of pseudovirus (125 infectious units); this mixture was incubated for 1 hour at room temperature. 10,000 293T-ACE2 cells (Moore, et al., J Virol 78, 10628-10635. (2004)) and the HIV-1 p24CA antigen capture assay (Leidos Biomedical Research, Inc.) in 20 μL of media containing 15 μg/mL polybrene was then added to each well and incubated at 37° C. for an additional 60-72 hours.

Assay buffers described previously (Siebring-van Olst, et al., J Biomol Screen 18, 453-461. (2013)) were used to perform cell lysis, and luciferase expression was then measured with a Spectramax L luminometer (Molecular Devices). Background luminescence from cells-only sample wells was calculated and subtracted from each sample well. Neutralization percentage at a given serum or monoclonal IgG concentration was then calculated by dividing by the luminescence of wells containing only virus and cells. GraphPad Prism (version 9) was used to fit nonlinear regressions were to the data and used to calculate IC50 values for all serum samples with neutralization values greater than or equal to 80% at maximum concentration. NT50 values were then calculated by taking the reciprocal of the IC50 values.

### Quantification and Statistical Analysis

GraphPad Prism was also used to perform other statistical analyses: the Kruskal-Wallis non-parametric ANOVA with post hoc analysis using Dunn's test for multiple comparisons was used to compare multiple populations, and the Mann-Whitney U test was used to compare two populations without consideration of paired samples. A p value <0.05 was considered to be statistically significant.

### V. Engineered Immunogens Elicit Broad Anti-Sarbecovirus Responses in the Context of SARS-CoV-2 Spike Imprinting

In this study, we leveraged rational, structure-guided immunogen design with the primary goal of eliciting broad immunity against sarbecoviruses. All experiments were performed in the context of prior immunity elicited against the SARS-2 spike to simulate prior infection and/or vaccination. Additionally, we sought to begin formally interrogating the value of increasing scaffold diversity when grafting epitopes of interest by measuring the relationship between scaffold antigenic difference and the extent to which the antibody response was biased towards the grafted epitope of interest. We employed iterative designs to graft the SARS-2 receptor binding motif onto heterologous coronavirus scaffolds with varying degrees of antigenic distance from SARS-2. Using multimerized versions of these grafted immunogens, we successfully elicited broad immunity against sarbecoviruses in the context of prior imprinting with the SARS-2 spike. Additionally, we found that immunization regimens that preferentially presented the SARS-2 receptor binding motif relative to other epitopes displayed improved neutralization breadth.

### Results

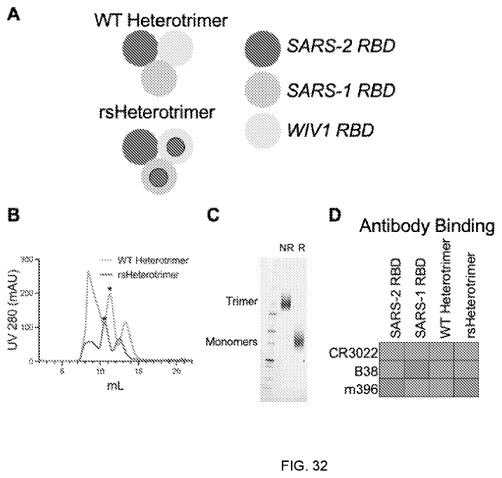

Immunization with receptor binding domains (RBDs) from multiple different sarbecoviruses has been previously shown to elicit a broadly neutralizing antibody response against both the sarbecoviruses with RBDs included in the immunogen and related sarbecoviruses. We aimed to engineer an immunogen that could present multiple RBDs on a single molecule while minimizing the epitopes introduced in the process of this multimerization. To that end, we used a previously described cystine-stabilized, hyperglycosylated GCN4 trimerization tag to combine the SARS-CoV-2 (SARS-2), SARS-CoV (SARS-1), and WIV1-CoV (WIV1) RBDs into a single “wild-type heterotrimer” immunogen (FIG. 32A).

In addition to characterizing the immunogenicity of the wildtype heterotrimer, we wanted to assess whether a heterotrimeric construct could be used to immune focus to an epitope of interest on the SARS-2 RBD. We selected the SARS-2 receptor binding motif (RBM) as a target. Focusing to this epitope may confer improved neutralization, as antibodies binding to RBM epitopes are likely to sterically interfere with ACE2 binding and subsequent cell entry. This “resurfaced heterotrimer” immunogen consists of the wild-type SARS-2 RBD in combination with the previously described resurfaced SARS-1 (rsSARS-1) and resurfaced WIV1 (rsWIV1) RBDs (FIG. 32A). As a result, this construct preferentially presents epitopes conserved across all three receptor binding domains: the SARS-2 RBM as well as conserved epitopes within the non-RBM portions of the SARS-2, SARS-1, and WIV1 RBDs. There is minimal conservation between the wild-type RBMs of the SARS-2, SARS-1, and WIV1 RBDs, so we hypothesized that immunizing with this resurfaced heterotrimeric construct would generate an increased antibody response to the SARS-2 RBM in comparison to the wild-type heterotrimer.

Both constructs were expressed in Expi293 cells from single plasmids encoding a polycistronic construct with three separate affinity tags, His, FLAG, and SBP, at the end of the trimerization tag for each of the constituent monomers. Cobalt, FLAG, and streptavidin resins were then used in sequence to isolate heterotrimeric constructs. Size-exclusion chromatography was performed following the cobalt resin purification step to remove aggregated and non-trimerized protein (FIG. 32B). Affinity tags were cleaved with HRV 3C protease following elution of the purified heterotrimers from streptavidin resin, and the constructs were re-purified via size-exclusion chromatography. The final cleaved product was run on an SDS-PAGE gel under reducing and non-reducing conditions to validate protein size and integrity of the trimerization tag (FIG. 32C). Bio-layer interferometry (BLI) was used to validate that antibody specificities of constituent monomers were appropriately retained following completed purification (FIG. 32D).

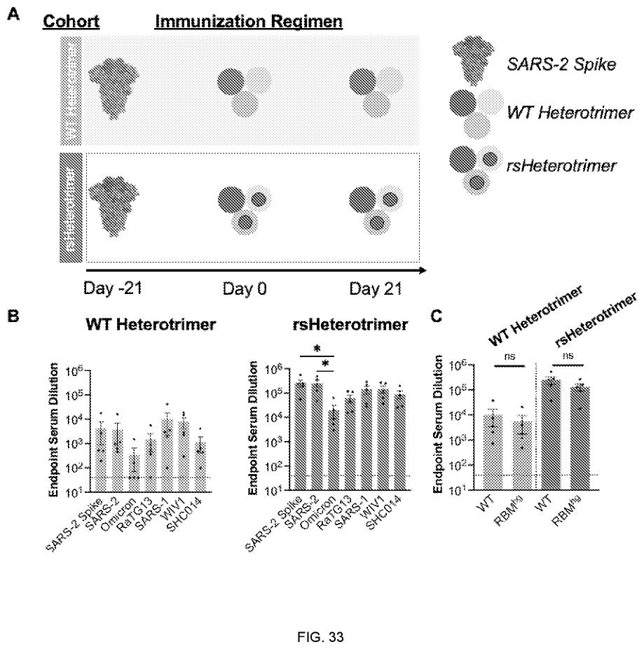

We then immunized C57BL/6 mice to evaluate the immunogenicity of these constructs in vivo. Given the approaching ubiquity of prior immunity to SARS-2, we primed both cohorts on day-21 with the recombinantly expressed SARS-2 spike ectodomain protein to simulate prior immunity elicited by infection or vaccination. Cohorts were then boosted twice on days 0 and 21. One cohort received the wild-type heterotrimer (“WT Heterotrimer cohort”), while the other received the resurfaced heterotrimer (“rsHeterotrimer cohort”) (FIG. 33A). Each cohort contained 5 mice, and sera was collected from each mouse at day 35 for evaluation of immunogenicity.

We performed ELISAs to measure serum antibody binding to a range of coronavirus RBDs (SARS-2, Omicron BA.1, SARS-1, WIV1, RaTG13, and SHC014) as well as the full SARS-2 spike protein (FIG. 33B). Overall, the WT Heterotrimer cohort had markedly lower endpoint antibody titers against these proteins, in the range of ˜10²-10⁴ in comparison to ˜10⁴-106 in the rsHeterotrimer cohort. However, both cohorts displayed a similar pattern of reactivity against the tested antigens, with titers being lowest against the Omicron RBD. Differences between titers against the Omicron RBD and the SARS-2 spike and RBD were statistically significant in the rsHeterotrimer Cohort. We also assessed the difference in serum antibody binding to the wild-type SARS-2 RBD in comparison to the previously described SARS-2 RBM^{hg} construct, which has 4 engineered putative N-linked glycosylation sites on the RBM that were designed to abrogate binding of RBM-directed antibodies. Consequently, a significant difference in the titers against these two constructs would indicate that the serum SARS-2 RBD-directed antibody response primarily targeted the SARS-2 RBM. Given that there is no significant difference between the wild-type SARS-2 RBD and the SARS-2 RBM^{hg} titers in either cohort, it is unlikely that the serum antibody responses are focused toward the SARS-2 RBM (FIG. 33C). Notably, the SARS-2 RBM^{hg} titers are slightly lower than the wild-type SARS-2 RBD titers, indicating that there likely are some RBM-directed antibodies within the serum.

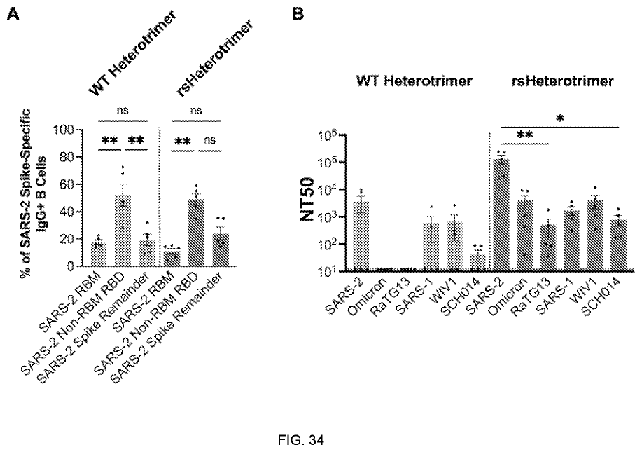

To complement our analysis of the serum response, we also analyzed antigen-specific memory B cells isolated from each cohort at day 42. We used the SARS-2 spike, SARS-2 RBD, and a version of the SARS-2 RBM^{hg} construct with two engineered putative N-linked glycosylation sites to bin antigen-specific memory B cells as binding to the SARS-2 RBM, the non-RBM portion of the SARS-2 RBD, or the non-RBD “remainder” of the SARS-2 spike using gating scheme. Memory B cells from both cohorts demonstrated similar patterns of reactivity, with the largest proportion of SARS-2 spike-specific B cells (˜50-60%) targeting the non-RBM portion of the SARS-2 RBD (FIG. 34A). These results are comparable to the pattern of memory B cell reactivity described in mice that were primed with the SARS-2 spike and boosted twice with homotrimeric SARS-2 RBD.

Given that the non-RBM portion of the SARS-2 RBD shares some conserved epitopes with related sarbecoviruses, we wanted to characterize the breadth of neutralization of the day 35 serum response from the WT Heterotrimer and rsHeterotrimer cohorts. Pseudovirus neutralization assays were performed against wild-type SARS-2, the SARS-2 Omicron BA.1 variant, RaTG13, SARS-1, WIV1, and SHC014. Sera from both cohorts displayed similar patterns of neutralization, with the highest titers against SARS-2 (FIG. 34B). This could reflect the effect of SARS-2 imprinting. Overall, titers were lower against the SARS-2 Omicron variant and related sarbecoviruses in comparison to wild-type SARS-2. However, these differences were only statistically significant for RaTG13 and SHC014 in the rsHeterotrimer cohort. Notably, the WT Heterotrimer cohort had no measurable neutralization against the SARS-2 Omicron variant and RaTG13. Titers in the WT Heterotrimer cohort were lower than those of the rsHeterotrimer cohort by about 1-2 orders of magnitude overall. This correlates with the difference in ELISA titers observed between the cohorts.

The results from both the WT Heterotrimer cohort and the rsHeterotrimer cohort demonstrate that an antibody response targeting predominantly non-RBM epitopes within the SARS-2 RBD can broadly bind and neutralize related sarbecoviruses. While the rsHeterotrimer cohort had higher ELISA and neutralization titers compared to the WT Heterotrimer cohort, this immunization regimen did not successfully focus to the SARS-2 RBM. We hypothesized that this could be the result of the similarity between the SARS-2, SARS-1, and WIV1 RBD epitopes outside of the RBM, given that there is at least 85% amino acid sequence conservation between each pair. In order to test this hypothesis, we attempted to graft the SARS-2 RBD onto more antigenically distinct heterologous coronavirus scaffolds. These scaffolds were selected from clade 2 of the sarbecovirus subgenus. The members of this clade were predominantly isolated from bats in southeast Asia, and these viruses do not use ACE2 as a host receptor.

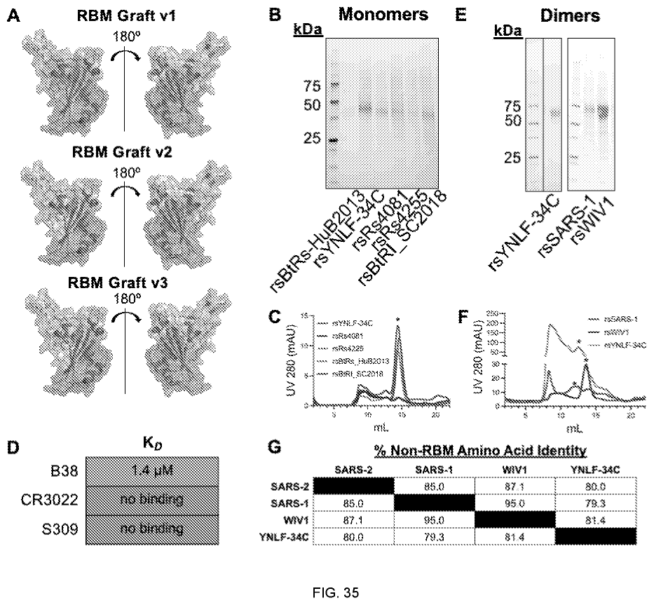

To graft the SARS-2 RBM onto these diverse scaffolds, we tested three different approaches. The first “v1” grafts used the same RBM boundaries as the previously published rsSARS-1 and rsWIV1 constructs: residues 437-507 (SARS-2 spike numbering) (FIG. 35A). Previously, a more extensive RBM graft has been shown by Letko et al., to confer ACE2 binding (Letko, M., et al., Nat Microbiol, (2020)). We replicated this graft in our “v2” construct, using SARS-2 RBM boundaries 401-507. The final “v3” construct incorporated the full v2 graft along with the SARS-2 RBD residues 348-352 in an effort to ensure that the full SARS-2 RBM was in contact with SARS-2-like residues.

We first attempted to express the v1 resurfaced constructs for 20 different clade 2 sarbecovirus RBDs. Of these 20 constructs, 5 showed evidence of expression in Expi 293F cells: rsBtRs-HuB2013, rsYNLF-34C, rsRs4081, rsRs4255, and rsBtRI_SC2018. However, additional biochemical analysis by size exclusion chromatography revealed that these proteins were almost completely aggregated. We then expressed the v2 and v3 resurfaced constructs for these 5 clade 2 sarbecovirus RBDs. For all 5 proteins, the v2 constructs attained the highest level of expression, with visible bands on SDS-Page gel analysis (FIG. 35B). Purified monomeric protein could be isolated via size exclusion chromatography (FIG. 35C). Of the 5 v2 constructs tested, rsYNLF-34C showed the highest level of monomeric protein expression from a standardized 25 mL transfection of Expi293F cells (FIG. 35C). We performed BLI to validate that rsYNLF-34C maintains binding to the RBM-directed Fab B38 with comparable affinity to the wild-type SARS-2 RBD. Furthermore, rsYNLF-34C did not have any detectable binding on BLI to the CR3022 or S309 Fabs. This indicates a lack of structural conservation in the non-RBM regions of the rsYNLF-34C RBD in comparison to the SARS-2 RBD. Consequently, we chose to advance this construct further for additional characterization as a candidate immunogen.

We constructed dimeric forms of the rsYNLF-34C, rsSARS-1, and rsWIV1 immunogens to improve immunogenicity as previously described for wild-type coronavirus RBDs. The rsWIV1 construct utilized the end-to-end dimerization approach previously described, while the rsSARS-1 and rsYNLF-34C dimers were constructed with 13 amino acid flexible linkers between the two RBDs to improved construct expression. Dimeric construct size was confirmed using SDS-PAGE gel analysis, and dimeric peaks could be isolated via size exclusion chromatography (FIG. 351E-F). The successful expression of these three dimeric constructs resulted in a toolkit that could be used to assess antigenic differences between scaffolds ranging from 79.3% to 95.0% amino acid identity (FIG. 35G).

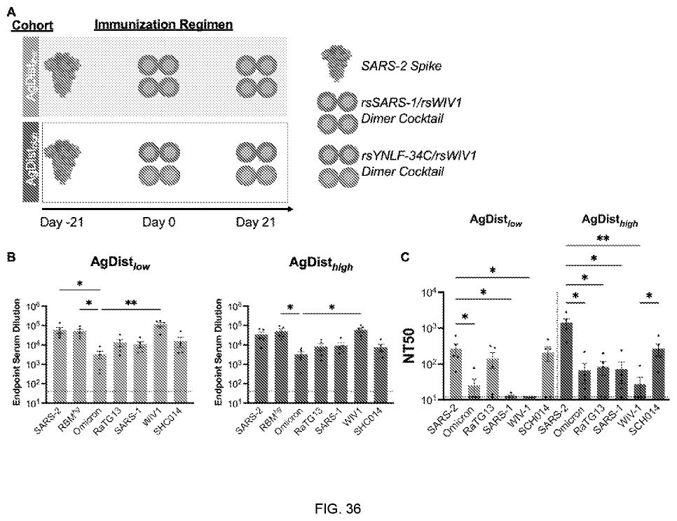

To test the hypothesis that increased antigenic distance between coronavirus RBD scaffolds would improve focusing to the SARS-2 RBM, we immunized two cohorts of five C57BL/6 mice (FIG. 36A). Both cohorts were primed with the SARS-2 spike ectodomain at day-21. The “AgDistlow” cohort was then boosted twice at days 0 and 21 with a cocktail of the rsSARS-1 and rsWIV1 dimers, which have 95.0% amino acid sequence identity in the non-RBM portion of the RBDs. The “AgDisthigh” cohort was boosted twice at days 0 and 21 with a cocktail of the rsYNLF-34C and rsWIV1 dimers, which have 81.4% amino acid sequence identity in the non-RBM portion of the RBDs. Serum was collected from both cohorts at day 35, and ELISAs were performed against a variety of coronavirus proteins (FIG. 36B). Neither cohort showed a significant difference in titers against the wild-type SARS-2 RBD and the SARS-2 RBM^{hg} construct, indicating a lack of serum antibody response focusing to the SARS-2 RBM. This indicates that additional scaffold antigenic diversity might be required to achieve successful focusing.

Despite the lack of RBM focusing, sera from the AgDisthigh cohort showed detectable neutralization against all sarbecoviruses tested (FIG. 36C). This contrasts with the sera from the AgDistlow cohort, which showed little evidence of neutralization against the SARS-2 Omicron variant, SARS-1, and WIV1. Immune focusing to conserved RBM epitopes has been previously shown to confer potent neutralization across a broad range of sarbecoviruses, indicating that there may be some level of immune focusing in the AgDisthigh cohort even though this difference cannot be detected via ELISA.

The above-described results indicate that, despite a lack of focusing to the SARS-2 RBM, the resurfaced immunogens designed in this study successfully elicit a broadly neutralizing antibody response in the context of prior SARS-2 spike imprinting. These findings indicate that preferentially increasing the representation of the SARS-2 RBM within candidate immunogens, such as the resurfaced heterotrimer, or immunization regimens, like the rsWIV1/rsYNLF-34C dimer cocktail, may improve neutralization breadth.

The above results were obtained using the following materials and methods.

### Materials and Methods

### Protein Expression and Purification

The SARS-2 spike plasmid contained a Foldon trimerization domain and C-terminal HRV 3C-cleavable 6×His and 2× Strep II tags. RBD designs were based on the following sequences: SARS-2 RBD (Genbank MN975262.1), SARS-1 RBD (Genbank ABD72970.1), WIV1 RBD (Genbank AGZ48828.1), RaTG13 RBD (Genbank QHR63300.2), SHC014 RBD (Genbank QJE50589.1). Codon optimization was performed using Integrated DNA Technologies, and gblock constructs were purchased and cloned into the pVRC expression vector. Proteins included a C-terminal HRV 3C-cleavable 8×His tag, as well as SBP tags in the monomeric and dimeric RBDs and a previously published hyperglycosylated, cystine-stabilized trimerization tag in the trimeric RBDs.

Constructs were expressed using Expifectamine transfection reagents in Expi293F cells (ThermoFisher) according to the manufacturer's protocol. After 5-7 days, transfections were harvested, and supernatants were purified via immobilized metal affinity chromatography using Cobalt-TALON resin (Takara). Eluted proteins were further purified by size-exclusion chromatography using a Superdex 200 Increase 10/300 GL column (Cytiva) in PBS (Corning). Relevant fractions were pooled. Heterotrimeric proteins were further purified using anti-FLAG resin (Genscript) and eluted with 160 μg/mL FLAG peptide (APExBIO) in Tris buffered saline followed by streptavidin resin (Pierce) eluted with 4 mM biotin (Sigma) in HEPES buffer.

HRV 3C protease (ThermoScientific) was used to remove purification tags prior to immunizations. Cleaved protein was re-purified using Cobalt-TALON resin and size exclusion chromatography to separate uncleaved protein, protease, and cleaved tags from cleaved protein.

### Immunizations

Immunizations were performed in C57BL/6 female mice aged 6-10 weeks (Charles River Laboratories). Each mouse received 20 μg of protein adjuvanted with 50% w/v Sigma Adjuvant System in 100 μL of inoculum via the intraperitoneal route. Immunizations occurred at days-21, 0, and 21, with serum characterization occurring with day 35 samples. Flow cytometry occurred between day 35 and 42. All experiments were conducted with institutional IACUC approval (MGH protocol 2014N000252).

### Flow Cytometry

Single cell suspensions of mice spleens were generated using a 70 μm cell strainer. Samples were treated with ACK lysis buffer, washed with PBS, and stained with the previously described B and T cell staining panel composed of CD3-BV786 (BioLegend), CD19-BV421 (BioLegend), IgM-BV605 (BioLegend), and IgG-PerCP/Cy5.5 (BioLegend) [7, 32]. At the same time, cells were labelled with streptavidin-conjugated fluorophores bound to the SBP-tagged proteins as follows: SARS-CoV-2 RBD-APC/Cy7 (streptavidin-APC/Cy7 from BioLegend), WIV1 RBD-BV650 (streptavidin-BV650 from BioLegend), SARS-CoV-2 spike-StreptTactin PE (StrepTactin PE from IBA Lifesciences), and SARS-CoV-2 spike-StreptTactin APC (StrepTactin APC from IBA Lifesciences). Labelling of SBP-tagged proteins involved the addition of streptavidin conjugates to a molar ratio of probe to streptavidin valency of 1:1 such that a final conjugated probe concentration of 0.1 μg/mL was achieved. Cells were then washed and stained with Aqua Live/Dead amine-reactive dye (0.025 mg/mL). Flow cytometry was performed using a BD FACSAria Fusion cytometer (BD Biosciences), and data was analyzed using FlowJo (version 10).

### Serum ELISAs

Serum ELISAs were performed as described herein. Briefly, plates were coated with 100 μL of protein at 5 μg/mL overnight at 4° C., then blocked with 150 μL of 1% BSA in PBS-Tween for 1 hour at room temperature. Sera was added at a starting dilution of 1:40 with a serial 5-fold dilution and incubated for 90 minutes at room temperature. Plates were washed, and 150 μL of HRP-conjugated anti-mouse IgG (Abcam) was added at 1:20,000. After a 1 hour secondary incubation at room temperature, plates were washed and 150 μL of 1× ABTS development solution (ThermoFisher) was added. Plates were developed for 30 minutes at room temperature before stopping with 100 μL of 1% SDS to read on a SpectraMaxiD3 plate reader (Molecular Devices) for absorbance at 405 nm.

### Pseudovirus Neutralization Assay

Serum pseudovirus neutralization assays were performed as described herein. Briefly, pseudotyped lentiviral particles were generated via transient transfection of 293T cells and tittered using 293T-ACE2 cells and the HIV-1 p24CA antigen capture assay (Leidos Biomedical Research, Inc.). Assays were performed using a Tecan Fluent Automated Workstation and 384-well plates (Grenier), and sera were initially diluted 1:3 with subsequent serial 3-fold dilutions. Each well received 20 μL each of sera and pseudovirus (125 infectious units), and plates were incubated 1 hour at room temperature. 10,000 293T-ACE2 cells in 20 μL of media with 15 μg/mL polybrene was added, and additional incubation occurred at 37° C. for 60-72 hours. Cells were lysed and luciferase expression was quantified on a Spectramax L luminometer (Molecular Devices). Neutralization percentage was calculated for each serum concentration by deducting background luminescence from cells-only wells and dividing by the luminescence of wells containing cells and virus. GraphPad Prism (version 9) was used to fit nonlinear regressions, and IC50 values were calculated for samples with neutralization values that were at least 80% at maximum serum concentration. Reciprocal IC50 values were used to obtain NT50 values.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the following numbered paragraphs.
1. A monomeric immunogen comprising one receptor binding domain of a Coronavirus spike protein, wherein the receptor binding domain comprises at least 4 non-native, N-linked, glycosylation sites.
2. The monomeric immunogen of claim 1, wherein the receptor binding domain of a Coronavirus spike protein comprises a SARS-CoV-2, SARS-1, WIV1, MERS, Rs_672, Rp_Shaanxi2011, Cp_Yunnan2011, BtRf-HeB2013, BtRf-SX2013, BtRs-HuB2013, BtRs-GX2013, BtRs-YN2013, Longquan-140, YNLF_31C, YNLF_34C, As6526, Rs4081, Rs4237, Rs4247, Rs4255, BtRI_SC2018, BtRs_YN2018A, BIRS_YN2018C, BtRs_YN2018D, HKU9, HKU3-1, HKU4, HKU5, RaTG13, or SHC014 receptor binding domain.
3. The monomeric immunogen of claim 1, wherein the receptor binding domain comprises a heterologous receptor binding motif.
4. The monomeric immunogen of claim 3, wherein the heterologous receptor binding motif comprises at least 2 non-native, N-linked, glycosylation sites.
5. The monomeric immunogen of claim 1, wherein the heterologous receptor binding motif of a Coronavirus is the ACE2 receptor binding motif of SARS-CoV-2 or a variant thereof.
6. The monomeric immunogen of claim 5, wherein the ACE2 receptor binding motif amino acid sequence comprises NSNNLDSKVGGNYNYLYRLFRKSNLKPFERDISTEIYQAGSTPCNGVEGENCYFPLQSYGFQPTNGVGY QPY.
7. The monomeric immunogen of claim 5, wherein the SARS-CoV-2 variant is Alpha (B.1.1.7, Q.1-Q.8), Beta (B.1.351, B.1.351.2, B.1.351.3), Gamma (P.1, P.1.1, P.1.2), Epsilon (B.1.427, B.1.429), Eta (B.1.525), lota (B.1.526), Kappa (B.1.617.1), Zeta (P.2), Mu (B.1.621, B.1.621.1) or Delta (B.1.617.2, AY.1 sublineages).
8. The monomeric immunogen of claim 5, wherein the SARS-CoV-2 variant is Alpha (B.1.1.7, Q.1-Q.8) and the ACE2 receptor binding motif amino acid sequence comprises NSNNLDSKVGGNYNYLYRLFRKSNLKPFERDISTEIYQAGSTPCNGVEGENCYFPLQSYGFQPTYGVGY QPY.
9. The monomeric immunogen of claim 5, wherein the SARS-CoV-2 variant is Beta (B.1.351, B.1.351.2, B.1.351.3), and the ACE2 receptor binding motif amino acid sequence NSNNLDSKVGGNYNYLYRLFRKSNLKPFERDISTEIYQAGSTPCNGVKGFNCYFPLQSYGFQPTYGVGY QPY.
10. The monomeric immunogen of claim 5, wherein the SARS-CoV-2 variant is Gamma (P.1, P.1.1, P.1.2), and the ACE2 receptor binding motif amino acid sequence comprises NSNNLDSKVGGNYNYLYRLFRKSNLKPFERDISTEIYQAGSTPCNGVKGFNCYFPLQSYGFQPTYGVGY QPY.
11. The monomeric immunogen of claim 5, wherein the SARS-CoV-2 variant is Epsilon (B.1.427, B.1.429), and the ACE2 receptor binding motif amino acid sequence comprises NSNNLDSKVGGNYNYRYRLFRKSNLKPFERDISTEIYQAGSTPCNGVEGFNCYFPLQSYGFQPTNGVGY QPY.
12. The monomeric immunogen of claim 5, wherein the SARS-CoV-2 variant is Eta (B.1.525), and the ACE2 receptor binding motif amino acid sequence comprises NSKNLDSKVGGNYNYLFRLFRKSNLKPFERDISTEIYQAGSTPCNGVKGFNCYFPLQSYGFQPTNGVGY QPY.
13. The monomeric immunogen of claim 5, wherein the SARS-CoV-2 variant is lota (B.1.526), and the ACE2 receptor binding motif amino acid sequence comprises NSNNLDSKVGGNYNYLYRLFRKSNLKPFERDISTEIYQAGSTPCNGVKGFNCYFPLQSYGFQPTNGVGY QPY.
14. The monomeric immunogen of claim 5, wherein the SARS-CoV-2 variant is Kappa (B.1.617.1), and the ACE2 receptor binding motif amino acid sequence comprises NSNNLDSKVGGNYNYRYRLFRKSNLKPFERDISTEIYQAGSTPCNGVQGFNCYFPLQSYGFQPTNGVGY QPY.
15. The monomeric immunogen of claim 5, wherein the SARS-CoV-2 variant is Zeta (P.2), Mu (B.1.621, B.1.621.1), and the ACE2 receptor binding motif amino acid sequence comprises NSNNLDSKVGGNYNYLYRLFRKSNLKPFERDISTEIYQAGSTPCNGVKGFNCYFPLQSYGFQPTNGVGY QPY.
16. The monomeric immunogen of claim 5, wherein the SARS-CoV-2 variant is Delta (B.1.617.2, AY.1), and the ACE2 receptor binding motif amino acid sequence comprises NSNNLDSKVGGNYNYRYRLFRKSNLKPFERDISTEIYQAGSKPCNGVEGFNCYFPLQSYGFQPTNGVGY QPY.
17. The monomeric immunogen of claim 1, wherein the receptor binding domain of a Coronavirus spike protein comprises a receptor binding domain selected from a SARS-1 or WIV1 receptor binding domain and the heterologous receptor binding motif of a Coronavirus is the ACE2 receptor binding motif of SARS-CoV-2.
18. The monomeric immunogen of any one of claims 1-17, wherein the receptor binding motif comprises a receptor binding motif shown in Table 3.
19. A trimeric immunogen comprising a hyperglycosylated and cysteine-stabilized GCN4 transcriptional activator domain and three linked receptor binding domains of a Coronavirus spike protein, wherein each receptor binding domain comprises a heterologous receptor binding motif of a Coronavirus, and wherein each receptor binding domain comprises at least 4 non-native, N-linked, glycosylation sites.
20. The trimeric immunogen of claim 19, wherein the receptor binding domain of a Coronavirus spike protein comprises a SARS-CoV-2, SARS-1, WIV1, MERS, Rs_672, Rp_Shaanxi2011, Cp_Yunnan2011, BtRf-HeB2013, BtRf-SX2013, BtRs-HuB2013, BtRs-GX2013, BtRs-YN2013, Longquan-140, YNLF_31C, YNLF_34C, As6526, Rs4081, Rs4237, Rs4247, Rs4255, BtRI_SC2018, BtRs_YN2018A, BtRS_YN2018C, BtRs_YN2018D, HKU9, HKU3-1, HKU4, HKU5, RaTG13, or SHC014 receptor binding domain.
21. The trimeric immunogen of claim 19, wherein the heterologous receptor binding motif of a Coronavirus is the ACE2 binding motif of SARS-CoV-2 or a variant thereof.
22. The trimeric immunogen of claim 19, wherein the receptor binding domain of a Coronavirus spike protein comprises a receptor binding domain selected from a SARS-1 or WIV1 receptor binding domain and the heterologous receptor binding motif of a Coronavirus is the ACE2 receptor binding motif of SARS-CoV-2.
23. A method of preventing a coronavirus infection in a subject or reducing the severity thereof, the method comprising: administering to the subject an effective amount of the monomeric immunogen of claim 1, or the trimeric immunogen of claim 19, or a combination thereof of the monomeric and trimeric immunogens, and a pharmaceutically acceptable carrier, thereby preventing the coronavirus infection in the subject or reducing the severity thereof.
24. The method of claim 20, wherein the subject has previously been infected with SARS-CoV-2.
25. The method of claim 20, wherein the subject has previously been vaccinated against Covid-19.
26. The method of claim 20, wherein the subject has antibodies against SARS-CoV-2.
27. The method of claim 20, wherein two or more trimeric immunogens are administered to the subject.
28. The method of claim 27, wherein three different trimeric immunogens are administered to the subject.
29. A polypeptide comprising

RVQPTESIVRFPNITNLCPFGEVFNATRFASVYAWNRSRISNCVA
DYSVLYNSSSFSTFKCYGVNATKLNDLCFTNVYADSFVIRGDEVR
QIAPGQTGKIADYNYKLPDDFTGCVIAWNSNNNDSKVGGNYNYLY
RLFRKSNLKPFERDNSTEIYQAGSTPCNGVEGFNCYFPLQSYGFQ
PTNGVGYQPYRVVVLSFELNHSPATVCGPKKGAGSSGSGRMKQIE
DKIENITSKIYNITNEIARIKKCCGNRT.

30. A polypeptide comprising

RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYSYLY
RLFRKSNLKPFERDISTEIYQNGSTPCNGVEGFNCYFPLQNYSFQ
PTNGTGYQPYRVVVLSFELLHAPATVCGPKKGAGSSGSGRMKQIE
DKIENITSKIYNITNEIARIKKCCGNRT.

31. A polypeptide comprising

RVAPSKEVVRFPNITNLCPFWEVFNATTFPSVYAWNRSRISNCVA
DYSVLYNSTSFSTFACYGVNATALNDLCFSNVYADSFVVKGDDVR
QIAPGQTGVIADYNYKLPDDFRGCVLAWNSNNNDSKVGGNYNYLY
RLFRKSNLKPFERDNSTEIYQAGSTPCNGVEGFNCYFPLQSYGFQ
PTNGVGYQPYRVVVLSFELNNSPATVCGPKLGAGSSGSGRMKQIE
DKIENITSKIYNITNEIARIKKCCGNRT.

32. A polypeptide comprising

RVVPSGDVVRFPNITNLCDFGEVFNATKFPSVYAWNRSKISNCVA
DYSVLYNSTFFSTFACYRVNATKLNDLCFSNVYADSFVVKGDDVR
QIAPGQTGVIADYNYKLPDDFKGCVLAWNSNNNDSKVGGNYNYLY
RLFRKSNLKPFERDNSTEIYQAGSTPCNGVEGFNCYFPLQSYGFQ
PTNGVGYQPYRVVVLSFELNNSPATVCGPKLGAGSSGSGRMKQIE
DKIENITSKIYNITNEIARIKKCCGNRT.

33. A polypeptide of any one of claims 29-32, further comprising SGGLEVLFQGPGSSHHHHHHHH.
34. A nucleic acid comprising an open reading frame that encodes an immunogen of any one of claims 1-22 or a polypeptide of any one of claims 29-33.
35. The nucleic acid of claim 34, wherein the nucleic acid is a messenger RNA.
36. A composition comprising the nucleic acid of claim 34 or 35.
37. A composition comprising a lipid nanoparticle and at least one mRNA of claim 35.
38. A method of preventing a coronavirus infection in a subject or reducing the severity thereof, the method comprising: administering to the subject an effective amount of the polypeptide of claims 29-33, or the composition of claim 36 or 37 and a pharmaceutically acceptable carrier, thereby preventing the coronavirus infection in the subject or reducing the severity thereof.
39. The method of claim 38, wherein the polypeptide is SARS-2^{hg}.
40. The method of claim 39, wherein the polypeptide is a trimer.

What is claimed is:

1. A monomeric immunogen comprising one receptor binding domain of a Coronavirus spike protein, wherein the receptor binding domain comprises at least 4 non-native, N-linked, glycosylation sites.

2. The monomeric immunogen of claim 1, wherein the receptor binding domain 5, 6, 7, or 9 non-native, N-linked, glycosylation sites.

3. The monomeric immunogen of claim 1, wherein the receptor binding domain of a Coronavirus spike protein comprises a SARS-CoV-2, SARS-1, WIV1, MERS, Rs_672, Rp_Shaanxi2011, Cp_Yunnan2011, BtRf-HeB2013, BtRf-SX2013, BtRs-HuB2013, BtRs-GX2013, BtRs-YN2013, Longquan-140, YNLF_31C, YNLF_34C, As6526, Rs4081, Rs4237, Rs4247, Rs4255, BtRI_SC2018, BtRs_YN2018A, BtRS_YN2018C, BtRs_YN2018D, HKU9, HKU3-1, HKU4, HKU5, RaTG13, or SHC014 receptor binding domain.

4. The monomeric immunogen of claim 1, wherein the receptor binding domain comprises a heterologous receptor binding motif.

5. The monomeric immunogen of claim 4, wherein the heterologous receptor binding motif comprises at least 2 non-native, N-linked, glycosylation sites.

6. The monomeric immunogen of claim 4, wherein the heterologous receptor binding motif of a Coronavirus is the ACE2 receptor binding motif of SARS-CoV-2 or a variant thereof.

7. The monomeric immunogen of claim 6, wherein the ACE2 receptor binding motif amino acid sequence comprises
(SEQ ID NO: 1)
NSNNLDSKVGGNYNYLYRLFRKSNLKPFERDISTEIYQAGSTPCNGVEGF
NCYFPLQSYGFQPTNGVGYQPY.

8. The monomeric immunogen of claim 6, wherein the SARS-CoV-2 variant is Alpha (B.1.1.7, Q.1-Q.8), Beta (B.1.351, B.1.351.2, B.1.351.3), Gamma (P.1, P.1.1, P.1.2), Epsilon (B.1.427, B.1.429), Eta (B.1.525), lota (B.1.526), Kappa (B.1.617.1), Zeta (P.2), Mu (B.1.621, B.1.621.1) or Delta (B.1.617.2, AY.1 sublineages).

9. The monomeric immunogen of claim 6, wherein the SARS-CoV-2 variant is Alpha (B.1.1.7, Q.1-Q.8) and the ACE2 receptor binding motif amino acid sequence comprises
(SEQ ID NO: 2)
NSNNLDSKVGGNYNYLYRLFRKSNLKPFERDISTEIYQAGSTPCNGVEGF
NCYFPLQSYGFQPTYGVGYQPY.

10. The monomeric immunogen of claim 6, wherein the SARS-CoV-2 variant is Beta (B.1.351, B.1.351.2, B.1.351.3), and the ACE2 receptor binding motif amino acid sequence
(SEQ ID NO: 3)
NSNNLDSKVGGNYNYLYRLFRKSNLKPFERDISTEIYQAGSTPCNGVKGF
NCYFPLQSYGFQPTYGVGYQPY.

11. The monomeric immunogen of claim 6, wherein the SARS-CoV-2 variant is Gamma (P.1, P.1.1, P.1.2), and the ACE2 receptor binding motif amino acid sequence comprises
(SEQ ID NO: 4)
NSNNLDSKVGGNYNYLYRLFRKSNLKPFERDISTEIYQAGSTPCNGVKGF
NCYFPLQSYGFQPTYGVGYQPY.

12. The monomeric immunogen of claim 6, wherein the SARS-CoV-2 variant is Epsilon (B.1.427, B.1.429), and the ACE2 receptor binding motif amino acid sequence comprises
(SEQ ID NO: 5)
NSNNLDSKVGGNYNYRYRLFRKSNLKPFERDISTEIYQAGSTPCNGVEGF
NCYFPLQSYGFQPTNGVGYQPY.

13. The monomeric immunogen of claim 6, wherein the SARS-CoV-2 variant is Eta (B.1.525), and the ACE2 receptor binding motif amino acid sequence comprises
(SEQ ID NO: 6)
NSKNLDSKVGGNYNYLFRLFRKSNLKPFERDISTEIYQAGSTPCNGVKGF
NCYFPLQSYGFQPTNGVGYQPY.

14. The monomeric immunogen of claim 6, wherein the SARS-CoV-2 variant is lota (B.1.526), and the ACE2 receptor binding motif amino acid sequence comprises
(SEQ ID NO: 7)
NSNNLDSKVGGNYNYLYRLFRKSNLKPFERDISTEIYQAGSTPCNGVKGF
NCYFPLQSYGFQPTNGVGYQPY.

15. The monomeric immunogen of claim 6, wherein the SARS-CoV-2 variant is Kappa (B.1.617.1), and the ACE2 receptor binding motif amino acid sequence comprises
(SEQ ID NO: 8)
NSNNLDSKVGGNYNYRYRLFRKSNLKPFERDISTEIYQAGSTPCNGVQGF
NCYFPLQSYGFQPTNGVGYQPY.

16. The monomeric immunogen of claim 6, wherein the SARS-CoV-2 variant is Zeta (P.2), Mu (B.1.621, B.1.621.1), and the ACE2 receptor binding motif amino acid sequence comprises
(SEQ ID NO: 9)
NSNNLDSKVGGNYNYLYRLFRKSNLKPFERDISTEIYQAGSTPCNGVKGF
NCYFPLQSYGFQPTNGVGYQPY.

17. The monomeric immunogen of claim 6, wherein the SARS-CoV-2 variant is Delta (B.1.617.2, AY.1), and the ACE2 receptor binding motif amino acid sequence comprises
(SEQ ID NO: 10)
NSNNLDSKVGGNYNYRYRLFRKSNLKPFERDISTEIYQAGSKPCNGVEGF
NCYFPLQSYGFQPTNGVGYQPY.

18. The monomeric immunogen of claim 1, wherein the receptor binding domain of a Coronavirus spike protein comprises a receptor binding domain selected from a SARS-1 or WIV1 receptor binding domain and the heterologous receptor binding motif of a Coronavirus is the ACE2 receptor binding motif of SARS-CoV-2.

19. The monomeric immunogen of any one of claims 1-18, wherein the receptor binding motif comprises a receptor binding motif shown in Table 3.

20. A dimeric immunogen comprising two of the monomeric immunogens of any one of claims 1-19.

21. The dimer immunogen of claim 20, wherein the dimer is two monomers of rsWIV1.

22. The dimer immunogen of claim 20, wherein the dimer is two monomers of rsSARS-1.

23. The dimer immunogen of claim 20, wherein the dimer is two monomers of rsYNLF-34C.

24. The dimer immunogen of claim 20, wherein the dimer is two different monomers.

25. A trimeric immunogen comprising a hyperglycosylated and cysteine-stabilized GCN4 transcriptional activator domain and three linked receptor binding domains of a Coronavirus spike protein, wherein each receptor binding domain comprises a heterologous receptor binding motif of a Coronavirus, and wherein each receptor binding domain comprises at least 4 non-native, N-linked, glycosylation sites.

26. The trimer immunogen of claim 25, wherein the receptor binding domain comprises 5, 6, 7, or 9 non-native, N-linked glycosylation sites.

27. The trimeric immunogen of claim 25, wherein the receptor binding domain of a Coronavirus spike protein comprises a SARS-CoV-2, SARS-1, WIV1, MERS, Rs_672, Rp_Shaanxi2011, Cp_Yunnan2011, BtRf-HeB2013, BtRf-SX2013, BtRs-HuB2013, BtRs-GX2013, BtRs-YN2013, Longquan-140, YNLF_31C, YNLF_34C, As6526, Rs4081, Rs4237, Rs4247, Rs4255, BtRI_SC2018, BtRs_YN2018A, BtRS_YN2018C, BtRs_YN2018D, HKU9, HKU3-1, HKU4, HKU5, RaTG13, or SHC014 receptor binding domain.

28. The trimeric immunogen of claim 25, wherein the heterologous receptor binding motif of a Coronavirus is the ACE2-binding motif of SARS-CoV-2 or a variant thereof.

29. The trimeric immunogen of claim 25, wherein the receptor binding domain of a Coronavirus spike protein comprises a receptor binding domain selected from a SARS-1 or WIV1 receptor binding domain and the heterologous receptor binding motif of a Coronavirus is the ACE2 receptor binding motif of SARS-CoV-2.

30. A receptor binding domain comprising ferritin.

31. The receptor binding domain of claim 30, wherein the RBD is resurfaced.

32. The receptor binding domain of claim 30, wherein the RBD comprises rsWIV1 or rsSARS-1.

33. The receptor binding domain of claim 30, wherein the RBD is conjugated to ferritin.

34. A ferritin nanoparticle comprising an immunogen comprising at least one receptor binding domain of a Coronavirus spike protein, wherein the receptor binding domain comprises at least 2 non-native, N-linked, glycosylation sites.

35. The nanoparticle of claim 34, wherein the immunogen is monomeric, dimeric, or trimeric.

36. The nanoparticle of claim 34 comprising at least 3, 6, 9, or 12 RBDs.

37. The nanoparticle of claim 34, wherein the receptor binding domain comprises rsWIV1.

38. The nanoparticle of claim 34, wherein the receptor binding domain comprises rsSARS-1.

39. The nanoparticle of claim 34, wherein the receptor binding domain is conjugated to ferritin.

40. A method of preventing a coronavirus infection in a subject or reducing the severity thereof, the method comprising:
administering to the subject an effective amount of the ferritin nanoparticle of claims 34-39, or the composition of claims 30-33, and a pharmaceutically acceptable carrier, thereby preventing the coronavirus infection in the subject or reducing the severity thereof.

41. A method of preventing a coronavirus infection in a subject or reducing the severity thereof, the method comprising:
administering to the subject an effective amount of the monomeric immunogen of claim 1-19, the dimeric immunogen of claim 20-24, or the trimeric immunogen of claim 25-29, or a combination thereof of the monomeric and trimeric immunogens, and a pharmaceutically acceptable carrier, thereby preventing the coronavirus infection in the subject or reducing the severity thereof.

42. The method of claim 41, wherein the subject has previously been infected with SARS-CoV-2.

43. The method of claim 41, wherein the subject has previously been vaccinated against Covid-19.

44. The method of claim 41, wherein the subject has antibodies against SARS-CoV-2.

45. The method of claim 41, wherein two or more dimeric immunogens are administered to the subject.

46. The method of claim 45, wherein the dimeric immunogens are the same.

47. The method of claim 45, wherein the dimeric immunogens are different.

48. The method of claim 41, wherein two or more trimeric immunogens are administered to the subject.

49. The method of claim 48, wherein the trimeric immunogens are the same.

50. The method of claim 48, wherein three different trimeric immunogens are administered to the subject.

51. A polypeptide comprising
(SEQ ID NO: 11)
RVQPTESIVRFPNITNLCPFGEVFNATRFASVYAWNRSRISNCVADYS
VLYNSSSFSTFKCYGVNATKLNDLCFTNVYADSFVIRGDEVRQIAPGQ
TGKIADYNYKLPDDFTGCVIAWNSNNNDSKVGGNYSYLYRLFRKSNLK
PFERDNSTEIYQNGSTPCNGVEGFNCYFPLQNYSFQPTNGVGYQPYRV
VVLSFELNHSPATVCGPKKGAGSSGSGRMKQIEDKIENITSKIYNITN
EIARIKKCCGNRT.

52. A polypeptide comprising
(SEQ ID NO: 12)
RVQPTESIVRFPNITNLCPFGEVFNATRFASVYAWNRKRISNCVADYS
VLYNSASFSTFKCYGVSPTKLNDLCFTNVYADSFVIRGDEVRQIAPGQ
TGKIADYNYKLPDDFTGCVIAWNSNNLDSKVGGNYNYLYRLFRKSNLK
PFERDISTEIYQNGSTPCNGVEGFNCYFPLQSYGFQPTNGTGGYQPYR
VVVLSFELLHAPATVCGPKKGAGSSGSGRMKQIEDKIENITSKIYNIT
NEIARIKKCCGNRT.

53. A polypeptide comprising
(SEQ ID NO: 13)
RVQPTESIVRFPNITNLCPFGEVFNATRFASVYAWNRSRISNCVA
DYSVLYNSSSFSTFKCYGVNATKLNDLCFTNVYADSFVIRGDEVR
QIAPGQTGKIADYNYKLPDDFTGCVIAWNSNNNDSKVGGNYNYLY
RLFRKSNLKPFERDNSTEIYQAGSTPCNGVEGFNCYFPLQSYGFQ
PTNGVGYQPYRVVVLSFELNHSPATVCGPKKGAGSSGSGRMKQIE
DKIENITSKIYNITNEIARIKKCCGNRT.

54. A polypeptide comprising
(SEQ ID NO: 14)
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYSYLY
RLFRKSNLKPFERDISTEIYQNGSTPCNGVEGENCYFPLQNYSFQ
PTNGTGYQPYRVVVLSFELLHAPATVCGPKKGAGSSGSGRMKQIE
DKIENITSKIYNITNEIARIKKCCGNRT.

55. A polypeptide comprising
(SEQ ID NO: 15)
RVAPSKEVVRFPNITNLCPFWEVFNATTFPSVYAWNRSRISNCVA
DYSVLYNSTSFSTFACYGVNATALNDLCFSNVYADSFVVKGDDVR
QIAPGQTGVIADYNYKLPDDFRGCVLAWNSNNNDSKVGGNYNYLY
RLFRKSNLKPFERDNSTEIYQAGSTPCNGVEGFNCYFPLQSYGFQ
PTNGVGYQPYRVVVLSFELNNSPATVCGPKLGAGSSGSGRMKQIE
DKIENITSKIYNITNEIARIKKCCGNRT.

56. A polypeptide comprising
(SEQ ID NO: 16)
RVVPSGDVVRFPNITNLCDFGEVFNATKFPSVYAWNRSKISNCVA
DYSVLYNSTFFSTFACYRVNATKLNDLCFSNVYADSFVVKGDDVR
QIAPGQTGVIADYNYKLPDDFKGCVLAWNSNNNDSKVGGNYNYLY
RLFRKSNLKPFERDNSTEIYQAGSTPCNGVEGFNCYFPLQSYGFQ
PTNGVGYQPYRVVVLSFELNNSPATVCGPKLGAGSSGSGRMKQIE
DKIENITSKIYNITNEIARIKKCCGNRT.

57. A polypeptide of any one of claims 51-56, further comprising
(SEQ ID NO: 17)
SGGLEVLFQGPGSSHHHHHHHH.

58. A nucleic acid comprising an open reading frame that encodes an immunogen of any one of claims 1-29, a receptor binding domain of any one of claims 30-33, a nanoparticle of any one of claims 34-39, or a polypeptide of any one of claims 51-57.

59. The nucleic acid of claim 58, wherein the nucleic acid is a messenger RNA.

60. A composition comprising the nucleic acid of claim 58 or 59.

61. A composition comprising a lipid nanoparticle and at least one mRNA of claim 59.

62. A method of preventing a coronavirus infection in a subject or reducing the severity thereof, the method comprising:
administering to the subject an effective amount of the polypeptide of claims 51-57, or the composition of claim 60 or 61 and a pharmaceutically acceptable carrier, thereby preventing the coronavirus infection in the subject or reducing the severity thereof.

63. The method of claim 62, wherein the polypeptide is SARS-2^{hg}.

64. The method of claim 63, wherein the polypeptide is a trimer.

65. The method of claim 62, wherein the polypeptide is a dimer.

66. The polypeptide of claim 51, wherein the polypeptide having immunogenic activity has at least at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or even 100% sequence identity to the polypeptide of SEQ ID NO: 11.

67. The polypeptide of claim 52, wherein the polypeptide having immunogenic activity has at least at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or even 100% sequence identity to the polypeptide of SEQ ID NO: 12.

68. The polypeptide of claim 53, wherein the polypeptide having immunogenic activity has at least at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or even 100% sequence identity to the polypeptide of SEQ ID NO: 13.

69. The polypeptide of claim 54, wherein the polypeptide having immunogenic activity has at least at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or even 100% sequence identity to the polypeptide of SEQ ID NO: 14.

70. The polypeptide of claim 55, wherein the polypeptide having immunogenic activity has at least at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or even 100% sequence identity to the polypeptide of SEQ ID NO: 15.

71. The polypeptide of claim 56, wherein the polypeptide having immunogenic activity has at least at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or even 100% sequence identity to the polypeptide of SEQ ID NO: 16.


SEQUENCE LISTING (87 sequences)

SEQ ID NO: 1 - protein, 72 aa, synthetic construct
NSNNLDSKVGGNYNYLYRLFRKSNLKPFERDISTEIYQAGSTPCNGVEGFNCYFPLQSYGFQPTNGVGYQPY

SEQ ID NO: 2 - protein, 72 aa, synthetic construct
NSNNLDSKVGGNYNYLYRLFRKSNLKPFERDISTEIYQAGSTPCNGVEGFNCYFPLQSYGFQPTYGVGYQPY

SEQ ID NO: 3 - protein, 72 aa, synthetic construct
NSNNLDSKVGGNYNYLYRLFRKSNLKPFERDISTEIYQAGSTPCNGVKGFNCYFPLQSYGFQPTYGVGYQPY

SEQ ID NO: 4 - protein, 72 aa, synthetic construct; identical to SEQ ID NO: 3

SEQ ID NO: 5 - protein, 72 aa, synthetic construct
NSNNLDSKVGGNYNYRYRLFRKSNLKPFERDISTEIYQAGSTPCNGVEGFNCYFPLQSYGFQPTNGVGYQPY

SEQ ID NO: 6 - protein, 72 aa, synthetic construct
NSKNLDSKVGGNYNYLFRLFRKSNLKPFERDISTEIYQAGSTPCNGVKGFNCYFPLQSYGFQPTNGVGYQPY

SEQ ID NO: 7 - protein, 72 aa, synthetic construct
NSNNLDSKVGGNYNYLYRLFRKSNLKPFERDISTEIYQAGSTPCNGVKGFNCYFPLQSYGFQPTNGVGYQPY

SEQ ID NO: 8 - protein, 72 aa, synthetic construct
NSNNLDSKVGGNYNYRYRLFRKSNLKPFERDISTEIYQAGSTPCNGVQGFNCYFPLQSYGFQPTNGVGYQPY

SEQ ID NO: 9 - protein, 72 aa, synthetic construct; identical to SEQ ID NO: 7

SEQ ID NO: 10 - protein, 72 aa, synthetic construct
NSNNLDSKVGGNYNYRYRLFRKSNLKPFERDISTEIYQAGSKPCNGVEGFNCYFPLQSYGFQPTNGVGYQPY

SEQ ID NO: 11 - protein, 253 aa, synthetic construct
RVQPTESIVRFPNITNLCPFGEVFNATRFASVYAWNRSRISNCVADYSVLYNSSSFSTFKCYGVNATKLNDLCFTNVYADSFVIRGDEVRQIAPGQTGKIADYNYKLPDDFTGCVIAWNSNNNDSKVGGNYSYLYRLFRKSNLKPFERDNSTEIYQNGSTPCNGVEGFNCYFPLQNYSFQPTNGVGYQPYRVVVLSFELNHSPATVCGPKKGAGSSGSGRMKQIEDKIENITSKIYNITNEIARIKKCCGNRT

SEQ ID NO: 12 - protein, 253 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQNGSTPCNGVEGFNCYFPLQSYGFQPTNGTGYQPYRVVVLSFELLHAPATVCGPKKGAGSSGSGRMKQIEDKIENITSKIYNITNEIARIKKCCGNRT

SEQ ID NO: 13 - protein, 253 aa, synthetic construct
RVQPTESIVRFPNITNLCPFGEVFNATRFASVYAWNRSRISNCVADYSVLYNSSSFSTFKCYGVNATKLNDLCFTNVYADSFVIRGDEVRQIAPGQTGKIADYNYKLPDDFTGCVIAWNSNNNDSKVGGNYNYLYRLFRKSNLKPFERDNSTEIYQAGSTPCNGVEGFNCYFPLQSYGFQPTNGVGYQPYRVVVLSFELNHSPATVCGPKKGAGSSGSGRMKQIEDKIENITSKIYNITNEIARIKKCCGNRT

SEQ ID NO: 14 - protein, 253 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYSYLYRLFRKSNLKPFERDISTEIYQNGSTPCNGVEGFNCYFPLQNYSFQPTNGTGYQPYRVVVLSFELLHAPATVCGPKKGAGSSGSGRMKQIEDKIENITSKIYNITNEIARIKKCCGNRT

SEQ ID NO: 15 - protein, 253 aa, synthetic construct
RVAPSKEVVRFPNITNLCPFWEVFNATTFPSVYAWNRSRISNCVADYSVLYNSTSFSTFACYGVNATALNDLCFSNVYADSFVVKGDDVRQIAPGQTGVIADYNYKLPDDFRGCVLAWNSNNNDSKVGGNYNYLYRLFRKSNLKPFERDNSTEIYQAGSTPCNGVEGFNCYFPLQSYGFQPTNGVGYQPYRVVVLSFELNNSPATVCGPKLGAGSSGSGRMKQIEDKIENITSKIYNITNEIARIKKCCGNRT

SEQ ID NO: 16 - protein, 253 aa, synthetic construct
RVVPSGDVVRFPNITNLCDFGEVFNATKFPSVYAWNRSKISNCVADYSVLYNSTFFSTFACYRVNATKLNDLCFSNVYADSFVVKGDDVRQIAPGQTGVIADYNYKLPDDFKGCVLAWNSNNNDSKVGGNYNYLYRLFRKSNLKPFERDNSTEIYQAGSTPCNGVEGFNCYFPLQSYGFQPTNGVGYQPYRVVVLSFELNNSPATVCGPKLGAGSSGSGRMKQIEDKIENITSKIYNITNEIARIKKCCGNRT

SEQ ID NO: 17 - protein, 22 aa, synthetic construct
SGGLEVLFQGPGSSHHHHHHHH

SEQ ID NO: 18 - DNA, 354 bp, synthetic construct
  source 1..354: mol_type = other DNA
gaggtccagctgcagcagtctggacctgagctggtgaagcctggggcttcagtgaagatatcctgcaaggcttctggttactcattcactggctactacatgaactgggtgaagcaaagtcctgaaaagagccttgagtggattggagagattaatcctaactttggtggtactacctacaaccagaagttcaaggccaaggccacattgactgtagacaaatcctccagcacagcctacatgcagctcaagagcctgacatctgaggactctgcagtctattactgtgcaagatactatggtaacctctatgctatggactactggggtcaaggaacctcagtcaccgtctcc

SEQ ID NO: 19 - DNA, 321 bp, synthetic construct
  source 1..321: mol_type = other DNA
gacatcctgatgacccagtctccatcctccatgtctgtatctctgggagacacagtcagcatcacttgccatgcaagtcagggcattagcagtaatatagggtggttgcagcagaaaccagggaaatcatttaagggcctgatctatcatggaaccaacttggaagatggagttccatcaaggttcagtggcagtggatctggagcagcttattctctcaccatcagcagcctggaatctgaagattttgcagactattactgtgtacagtatactcattttccgtacacgttcggaggggggaccaagctggaaataaaa

SEQ ID NO: 20 - DNA, 354 bp, synthetic construct
  source 1..354: mol_type = other DNA
gaggtccagctgcagcagtctggacctgagctggtgaagcctggggcttcagtgaagatatcctgcaaggcttctggttactcatttaataactactacatgaactgggtgaagcagagtcctgaaaagagccttgagtggattggagagattaatcctaactctggttatacttcctacaaccagaagttcagggccaaggccacattgactgtagacaaatcctccaccacagcctacatgcagctcaagagcctgacatctgaggactctgcggtctattactgtgcaagatactttggtaacctctttgctatggacttctggggtcaaggaacctcagtcaccgtctcc

SEQ ID NO: 21 - DNA, 321 bp, synthetic construct
  source 1..321: mol_type = other DNA
gacatcctgatgacccaatctccatcctccatgtctgtatctctgggagacacagtcagcatcacttgccatgcaagtcagggcattggcagtaatatagggtggttgcagcagaaaccagggaaatcatttaagggcctgatctatcttggaaccaacttggaagatggagttccatcaaggttcagtggcagtggatctggagcagattattctctcaccatcagcagcctggaatctgaagattttgcagactattactgtgtacagtatgttcagtttccgtacacgttcggaggggggaccaagctggaaataaaa

SEQ ID NO: 22 - DNA, 357 bp, synthetic construct
  source 1..357: mol_type = other DNA
gaggtccagctgcagcagtctggacctgagctggtgaagcctggggcttcagtgaagatatcctgcaaggcttctggttactcattcactgactactacatgaactgggtgaagcaaagtcctgaaaagagccttgagtggattggagagattaatcctaacactggtggtactacctacaaccagaagttcaaggccaaggccacattgactgtagacaaatcctccagcacagcctacatgcagctcaagagcctgacatctgaggactctgcagtctattactgtgcaagatactatggtaacctctatgctatggactactggggtcaaggaacctcagtcaccgtctcctca

SEQ ID NO: 23 - DNA, 321 bp, synthetic construct
  source 1..321: mol_type = other DNA
gacatcctgatgacccaatctccatcctccatgtctgtatctctgggagacacagtcagcatcacatgccatgcaagtcagggcataagtagtaatatagggtggttgcagcagaaaccagggaaatcatttaagggcctgatctatcatggaaccaacttggaagatggagttccatcaaggttcagtggcagtggatctggagcagattattctctcaccatcagcagcctggaatctgaagattttgcagactattactgtgtacagtatgttcagtttccgtacacgctcggaggggggaccaagctggaaataaaa

SEQ ID NO: 24 - DNA, 363 bp, synthetic construct
  source 1..363: mol_type = other DNA
caggttcagctgcagcagtctggagctgagctggcgaggcctggggcttcagtgaagctgtcctgcaaggcttctggctaccccttcacaagctatggtataaactgggtgaagcagagaactggacagggccttgagtggattggagagatttatcctagaattggaaatacttactataatgagaagttcaagggcaaggccacactgactgcagacaaatcctccagcacagcgtacatggagttccgcagcctgacatctgaggactctgcggtctatttctgtgcaagatcgtggaatagtaactacggggagtactactttgactactggggccaaggcaccactctcacagtctcc

SEQ ID NO: 25 - DNA, 321 bp, synthetic construct
  source 1..321: mol_type = other DNA
gacattgtgatgacccagtctcacaaattcatgtccacatcaataggagacagggtcagcatcacctgcaaggccagtcacgatgtgagtactgctgtagcctggtatcaacaaaaaccagggcaatctcctaagttactgatttactgggcatccacccggcacactggagtccctgatcgcttcacaggcagtggatctgggacagattatactctcaccattagaagtgtgcaggcagaagacctggcactttattactgtcagcaacattatagcactccgtacacgttcggaggggggaccaagctggaaataaaa

SEQ ID NO: 26 - DNA, 342 bp, synthetic construct
  source 1..342: mol_type = other DNA
gaggtccagctgcagcagtctggacctgagctggtgaagcctggggcttcagtgaagatatcctgcaaggcttctggtttctcattcactggctactccatgaactggatgaaacaaagtcctgaaaagagccttgagtggattggagaaattaatcctaccactggtggtactacctacaaccagaagttcaaggccaaggccacattgactgtagacaaatcctccagcacagcctacatacaactcaagagcctgacatctgaggactctgcagtctattactgtgcaaggggccgggccgactactggggccaaggcaccactctcacagtctcctca

SEQ ID NO: 27 - DNA, 333 bp, synthetic construct
  source 1..333: mol_type = other DNA
gacattgtgctcacccaatctccagcttctttggctgtgtctctagggcagagagccaccatctcctgcagagccagtgaaagtgttgaatattatggcacaggtttagtgcagtggttccaacagaaaccaggacagccacccaaactcctcatctatgctgcctccaacgtggaatctggggtccctgccaggtttagtggcagtgggtctgggacagacttcagcctcaacatccattctgtggaggaggatgatattgcaatgtatttctgtcaccaaagtaggaagcttccgtggacgttcggtggaggcaccaagctggaaatcaaa

SEQ ID NO: 28 - protein, 118 aa, synthetic construct
EVQLQQSGPELVKPGASVKISCKASGYSFTGYYMNWVKQSPEKSLEWIGEINPNFGGTTYNQKFKAKATLTVDKSSSTAYMQLKSLTSEDSAVYYCARYYGNLYAMDYWGQGTSVTVS

SEQ ID NO: 29 - protein, 107 aa, synthetic construct
DILMTQSPSSMSVSLGDTVSITCHASQGISSNIGWLQQKPGKSFKGLIYHGTNLEDGVPSRFSGSGSGAAYSLTISSLESEDFADYYCVQYTHFPYTFGGGTKLEIK

SEQ ID NO: 30 - protein, 118 aa, synthetic construct
EVQLQQSGPELVKPGASVKISCKASGYSFNNYYMNWVKQSPEKSLEWIGEINPNSGYTSYNQKFRAKATLTVDKSSTTAYMQLKSLTSEDSAVYYCARYFGNLFAMDFWGQGTSVTVS

SEQ ID NO: 31 - protein, 107 aa, synthetic construct
DILMTQSPSSMSVSLGDTVSITCHASQGIGSNIGWLQQKPGKSFKGLIYLGTNLEDGVPSRFSGSGSGADYSLTISSLESEDFADYYCVQYVQFPYTFGGGTKLEIK

SEQ ID NO: 32 - protein, 119 aa, synthetic construct
EVQLQQSGPELVKPGASVKISCKASGYSFTDYYMNWVKQSPEKSLEWIGEINPNTGGTTYNQKFKAKATLTVDKSSSTAYMQLKSLTSEDSAVYYCARYYGNLYAMDYWGQGTSVTVSS

SEQ ID NO: 33 - protein, 107 aa, synthetic construct
DILMTQSPSSMSVSLGDTVSITCHASQGISSNIGWLQQKPGKSFKGLIYHGTNLEDGVPSRFSGSGSGADYSLTISSLESEDFADYYCVQYVQFPYTLGGGTKLEIK

SEQ ID NO: 34 - protein, 121 aa, synthetic construct
QVQLQQSGAELARPGASVKLSCKASGYPFTSYGINWVKQRTGQGLEWIGEIYPRIGNTYYNEKFKGKATLTADKSSSTAYMEFRSLTSEDSAVYFCARSWNSNYGEYYFDYWGQGTTLTVS

SEQ ID NO: 35 - protein, 107 aa, synthetic construct
DIVMTQSHKFMSTSIGDRVSITCKASHDVSTAVAWYQQKPGQSPKLLIYWASTRHTGVPDRFTGSGSGTDYTLTIRSVQAEDLALYYCQQHYSTPYTFGGGTKLEIK

SEQ ID NO: 36 - protein, 114 aa, synthetic construct
EVQLQQSGPELVKPGASVKISCKASGFSFTGYSMNWMKQSPEKSLEWIGEINPTTGGTTYNQKFKAKATLTVDKSSSTAYIQLKSLTSEDSAVYYCARGRADYWGQGTTLTVSS

SEQ ID NO: 37 - protein, 111 aa, synthetic construct
DIVLTQSPASLAVSLGQRATISCRASESVEYYGTGLVQWFQQKPGQPPKLLIYAASNVESGVPARFSGSGSGTDFSLNIHSVEEDDIAMYFCHQSRKLPWTFGGGTKLEIK

SEQ ID NO: 38 - protein, 167 aa, synthetic construct
SYCTPPYSVLQDPPQPVVWRRYMLYDCVFDFTVVVDSLPTHQLQCYGVSPRRLASMCYGSVTLDVMRINETHLNNLFNRVPDTFSLYNYALPDNFYGCLHAFYLNSTAPYAVANRFPIKPGGRQSNSAFIDTVINAAHYSPFSYVYGLAVITLKPAAGSKLVCPVAN

SEQ ID NO: 39 - protein, 234 aa, synthetic construct
EAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRFLSDDRTEVPQLVNANQYSPCVSIVPSTVWEDGDYYRKQLSPLEGGGWLVASGSTVAMTEQLQMGFGITVQYGTDTNSVCPKLEFANDTKIASQL

SEQ ID NO: 40 - protein, 192 aa, synthetic construct
RVSPTHEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLTSDENGVRTLSTYDFYPNVPIEYQATRVVVLSFELLNAPATVCGPKL

SEQ ID NO: 41 - protein, 192 aa, synthetic construct
RVSPTQEVVRFPNITNRCPFDKVFNATRFPSVYAWERTKISDCVADYTVLYNSTSFSTFKCYGVSPSKLIDLCFTSVYADTFLIRSSEVRQVAPGETGVIADYNYKLPDDFTGCVIAWNTANQDQGQYYYRSSRKEKLKPFERDLSSDENGVYTLSTYDFYPSVPLDYQATRVVVLSFELLNAPATVCGPKL

SEQ ID NO: 42 - protein, 192 aa, synthetic construct
RVSPSTEVIRFPNITNRCPFDRVFNASRFPSVYAWERTKISDCVADYTVLYNSTSFSTFKCYGVSPSKLIDLCFTSVYADTFLIRFSEVRQIAPGETGVIADYNYKLPDEFTGCVIAWNTANQDRGQYYYRSSRKTKLKPFERDLSSDENGVRTLSTYDFYPSVPLEYQATRVVVLSFELLNAPATVCGPKL

SEQ ID NO: 43 - protein, 192 aa, synthetic construct
RVSPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKQDVGSYFYRSHRSSKLKPFERDLSSEENGVRTLSTYDFNQYVPLEYQATRVVVLSFELLNAPATVCGPKL

SEQ ID NO: 44 - protein, 192 aa, synthetic construct; identical to SEQ ID NO: 43

SEQ ID NO: 45 - protein, 193 aa, synthetic construct
RVTPTQEVVRFPNITNRCPFDRVFNASRFPSVYAWERTKISDCVADYTVLYNSTSFSTFKCYGVSPSKLIDLCFTSVYADTFLIRSSEVRQVAPGETGVIADYNYKLPDDFTGCVIAWNTAKQDTGYYYYRSHRKTKLKPFERDLSSDDGNGVYTLSTYDFNPNVPVAYQATRVVVLSFELLNAPATVCGPKL

SEQ ID NO: 46 - protein, 193 aa, synthetic construct
RVSPTQEVVRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDTGNYYYRSHRKTKLKPFERDLSSDDGNGVYTLSTYDFNPNVPVAYQATRVVVLSFELLNAPATVCGPKL

SEQ ID NO: 47 - protein, 192 aa, synthetic construct
RVSPTREVVRFPNITNRCPFDSIFNASRFPSVYAWERTKISDCVADYTVLYNSTLFSTFKCYGVSPSKLIDLCFTSVYADTFLIRFSEVRQVAPGETGVIADYNYRLPDDFTGCVIAWNTANQDVGSYFYRSHRSTKLKPFERDLSSDENGVRTLSTYDFNPNVPLDYQATRVVVLSFELLNAPATVCGPKL

SEQ ID NO: 48 - protein, 193 aa, synthetic construct
RVSPTQEVIRFPNITNRCPFDKVFNVTRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDIGNYYYRSHRKTKLKPFERDLSSDDGNGVYTLSTYDFNPNVPVAYQATRVVVLSFELLNAPATVCGPKL

SEQ ID NO: 49 - protein, 192 aa, synthetic construct
RVAPVTEVVRFPNITNLCPFDKVFNATRFPSVYAWERTKISDCVADYTVFYNSTSFSTFNCYGVSPSKLIDLCFTSVYADTFLIRFSEVRQVAPGQTGVIADYNYKLPDDFTGCVIAWNTAKYDVGSYFYRSHRSSKLKPFERDLSSEENGARTLSTYDFNQNVPLEYQATRVVVLSFELLNAPATVCGPKL

SEQ ID NO: 50 - protein, 192 aa, synthetic construct; identical to SEQ ID NO: 49

SEQ ID NO: 51 - protein, 192 aa, synthetic construct
RVSPTQEVVRFPNITNRCPFDKVFNATRFPSVYAWERTKISDCVADYTVLYNSTSFSTFKCYGVSPSKLIDLCFTSVYADTFLIRSSEVRQVAPGETGVIADYNYKLPDDFTGCVIAWNTAQQDKGQYYYRSSRKTKLKPFERDLSSDENGVRTLSTYDFYPTVPIEYQATRVVVLSFELLNAPATVCGPKL

SEQ ID NO: 52 - protein, 192 aa, synthetic construct
RVSPTHEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLTSDENGVRTLSTYDFYPNVPIEYQATRVVVLSFELLNAPATVCGPKL

SEQ ID NO: 53 - protein, 192 aa, synthetic construct
RVSPTQEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLSSDENGVRTLSTYDFYPTVPIEYQATRVVVLSFELLNAPATVCGPKL

SEQ ID NO: 54 - protein, 193 aa, synthetic construct
RVSPTQEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDTGHYYYRSHRKTKLKPFERDLSSDDGNGVYTLSTYDFNPNVPVAYQATRVVVLSFELLNAPATVCGPKL

SEQ ID NO: 55 - protein, 192 aa, synthetic construct
RVSPTHEVIRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDQGQYYYRSSRKTKLKPFERDLSSDENGVRTLSTYDFYPTVPIEYQATRVVVLSFELLNAPATVCGPKL

SEQ ID NO: 56 - protein, 193 aa, synthetic construct
RVSPTQEVVRFPNITNRCPFDKVFNASRFPSVYAWERIKISDCVADYTVLYNSTSFSTFKCYGVSPSKLIDLCFTSVYADTFLIRSSEVRQVAPGETGVIADYNYKLPDDFTGCVIAWNTAKQDTGSYYYRSHRKTKLKPFERDLSSDDGNGVYTLSTYDFNPNVPVAYQATRVVVLSFELLNAPATVCGPKL

SEQ ID NO: 57 - protein, 193 aa, synthetic construct
RVSPTQEVVRFPNITNRCPFDKVFNASRFPNVYAWERTKISDCVADYTVLYNSTSFSTFKCYGVSPSKLIDLCFTSVYADTFLIRSSEVRQVAPGETGVIADYNYKLPDDFTGCVIAWNTAKQDTGHYYYRSHRKTKLKPFERDLSSDDGNGVYTLSTYDFNPNVPVAYQATRVVVLSFELLNAPATVCGPKL

SEQ ID NO: 58 - protein, 192 aa, synthetic construct; identical to SEQ ID NO: 40

SEQ ID NO: 59 - protein, 192 aa, synthetic construct; identical to SEQ ID NO: 40

SEQ ID NO: 60 - protein, 193 aa, synthetic construct
RVSPTQEVIRFPNITNRCPFDKVFNATRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKL

SEQ ID NO: 61 - protein, 234 aa, synthetic construct
EASATGTFIEQPNATECDFSPMLTGVAPQVYNFKRLVFSNCNYNLTKLLSLFAVDEFSCNGISPDSIARGCYSTLTVDYFAYPLSMKSYIRPGSAGNIPLYNYKQSFANPTCRVMASVLANVTITKPHAYGYISKCSRLTGANQDVETPLYINPGEYSICRDFSPGGFSEDGQVFKRTLTQFEGGGLLIGVGTRVPMTDNLQMSFIISVQYGTGTDSVCPMLDLGDSLTITNRL

SEQ ID NO: 62 - protein, 224 aa, synthetic construct
EASPRGEFIEQATTQECDFTPMLTGTPPPIYNFKRLVFTNCNYNLTKLLSLFQVSEFSCHQVSPSSLATGCYSSLTVDYFAYSTDMSSYLQPGSAGAIVQFNYKQDFSNPTCRVLATVPQNLTTITKPSNYAYLTECYKTSAYGKNYLYNAPGAYTPCLSLASRGFSTKYQSHSDGELTTTGYIYPVTGNLQMAFIISVQYGTDTNSVCPMQALRNDTSIEDKL

SEQ ID NO: 63 - protein, 210 aa, synthetic construct
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKL

SEQ ID NO: 64 - protein, 210 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKL

SEQ ID NO: 65 - protein, 211 aa, synthetic construct
RVQPTDSIVRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKK

SEQ ID NO: 66 - protein, 211 aa, synthetic construct
RVAPSKEVVRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLS

SEQ ID NO: 67 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 68 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKK

SEQ ID NO: 69 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKK

SEQ ID NO: 70 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKK

SEQ ID NO: 71 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKK

SEQ ID NO: 72 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSKNLDSKVGGNYNYLFRLFRKSNLKPFERDISTEIYQAGSTPCNGVKGFNCYFPLQSYGFQPTNGVGYQPYRVVVLSFELLHAPATVCGPKK

SEQ ID NO: 73 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRVVVLSFELLHAPATVCGPKK

SEQ ID NO: 74 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVQGFNCYFPLQSYGFQPTNGVGYQPYRVVVLSFELLHAPATVCGPKK

SEQ ID NO: 75 - protein, 211 aa, synthetic construct; identical to SEQ ID NO: 73

SEQ ID NO: 76 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKK

SEQ ID NO: 77 - protein, 211 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKK

SEQ ID NO: 78 - protein, 275 aa, synthetic construct
RVQPTESIVRFPNITNLCPFGEVFNATRFASVYAWNRSRISNCVADYSVLYNSSSFSTFKCYGVNATKLNDLCFTNVYADSFVIRGDEVRQIAPGQTGKIADYNYKLPDDFTGCVIAWNSNNNDSKVGGNYNYLYRLFRKSNLKPFERDNSTEIYQAGSTPCNGVEGFNCYFPLQSYGFQPTNGVGYQPYRVVVLSFELNHSPATVCGPKKGAGSSGSGRMKQIEDKIENITSKIYNITNEIARIKKCCGNRTSGGLEVLFQGPGSSHHHHHHHH

SEQ ID NO: 79 - protein, 275 aa, synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYSYLYRLFRKSNLKPFERDISTEIYQNGSTPCNGVEGFNCYFPLQNYSFQPTNGTGYQPYRVVVLSFELLHAPATVCGPKKGAGSSGSGRMKQIEDKIENITSKIYNITNEIARIKKCCGNRTSGGLEVLFQGPGSSHHHHHHHH

SEQ ID NO: 80 - protein, 275 aa, synthetic construct
RVAPSKEVVRFPNITNLCPFWEVFNATTFPSVYAWNRSRISNCVADYSVLYNSTSFSTFACYGVNATALNDLCFSNVYADSFVVKGDDVRQIAPGQTGVIADYNYKLPDDFRGCVLAWNSNNNDSKVGGNYNYLYRLFRKSNLKPFERDNSTEIYQAGSTPCNGVEGFNCYFPLQSYGFQPTNGVGYQPYRVVVLSFELNNSPATVCGPKLGAGSSGSGRMKQIEDKIENITSKIYNITNEIARIKKCCGNRTSGGLEVLFQGPGSSHHHHHHHH

SEQ ID NO: 81 - protein, 275 aa, synthetic construct
RVVPSGDVVRFPNITNLCDFGEVFNATKFPSVYAWNRSKISNCVADYSVLYNSTFFSTFACYRVNATKLNDLCFSNVYADSFVVKGDDVRQIAPGQTGVIADYNYKLPDDFKGCVLAWNSNNNDSKVGGNYNYLYRLFRKSNLKPFERDNSTEIYQAGSTPCNGVEGFNCYFPLQSYGFQPTNGVGYQPYRVVVLSFELNNSPATVCGPKLGAGSSGSGRMKQIEDKIENITSKIYNITNEIARIKKCCGNRTSGGLEVLFQGPGSSHHHHHHHH

SEQ ID NO: 82 - protein, 34 aa, synthetic construct
RMKQIEDKIENITSKIYNITNEIARIKKCCGNRT

SEQ ID NO: 83 - protein, 8 aa, synthetic construct
LEVLFQGP

SEQ ID NO: 84 - protein, 8 aa, synthetic construct
HHHHHHHH

SEQ ID NO: 85 - protein, 8 aa, synthetic construct
GAGSSGSG

SEQ ID NO: 86 - protein, 22 aa, synthetic construct
SGGLEVLFQGPGSSHHHHHHHH

SEQ ID NO: 87 - skipped